(19) United States
(12) Patent Application Publication
(10) Pub. No.: US 2024/0316020 A1
(43) Pub. Date: Sep. 26, 2024

(54) Novel Pyridine Compounds

(71) Applicants: ICAGEN, LLC, Durham, NC (US); HOFFMANN-LA ROCHE INC., Little Falls, NJ (US)

(72) Inventors: Mark CHAPMAN, Durham, NC (US); Nicholas Gareth Morse DAVIES, Durham, NC (US); Aaron GERLACH, Durham, NC (US); Sylvain LEBRETON, Tucson, AZ (US); Ingrid MECHIN, Durham, NC (US); David MOWREY, Durham, NC (US); Karthigeyan NAGARAJAN, Durham, NC (US); Anil NAIR, Durham, NC (US); Roger David NORCROSS, Aargau (CH); Roger Lluis REDONDO PENA, Basel (CH); Alena SAFAROVA, Durham, NC (US); Martin SMRCINA, Durham, NC (US)

(21) Appl. No.: 18/665,797

(22) Filed: May 16, 2024

Related U.S. Application Data
(63) Continuation of application No. PCT/US2022/050065, filed on Nov. 16, 2022.
(60) Provisional application No. 63/281,140, filed on Nov. 19, 2021.

(51) Int. Cl. A61K 31/4439; A61K 9/20; A61K 31/443; A61K 31/4433; C07D 213/38; C07D 213/64; C07D 405/12; C07D 405/14

(52) U.S. Cl. CPC A61K 31/4439; A61K 9/2009; A61K 9/2018; A61K 9/2054; A61K 9/2059; A61K 31/443; A61K 31/4433; C07D 213/38; C07D 213/64; C07D 405/12; C07D 405/14

(57) ABSTRACT

The invention provides new heterocyclic compounds having the general formula (I), or a solvate or a pharmaceutically acceptable salt thereof:

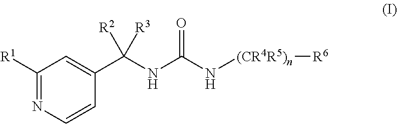

wherein R¹, R², R³, R⁴, R⁵, R⁶ and n are as defined herein, compositions including the compounds, processes of manufacturing the compounds and methods of using the compounds.

### FIELD OF THE INVENTION

The present invention relates to novel pyridine compounds useful as Kv7.2 enhancers (or positive modulators), their manufacture, pharmaceutical compositions, kits comprising the compounds, and their use as medicaments for the therapeutic and/or prophylactic treatment of a disorder, disease, or disability associated with Kv7.2. These disorders, diseases, or disabilities can be selected from behavioral disorders, mood disorders, neurodevelopmental disorders, intellectual disability, epilepsies, neurodegenerative diseases, pain, migraine, and tinnitus.

### BACKGROUND OF THE INVENTION

The potassium channel family 7, or Q, contains five proteins that in humans are encoded by the genes KCNQ1, KCNQ2, KCNQ3, KCNQ4, and KCNQ5. The KCNQ proteins form homo- and hetero-tetrameric channels that respond to membrane voltage changes and open to let potassium ions flow out of cell membranes. Homomeric Kv7.2 channels as well as heteromeric Kv7.2 and Kv7.3 channels have been investigated because of their unique distribution and their potential role as primary regulators of neuronal excitability in many CNS and PNS pathways (Wang et al., 1998). KCNQ2 channels control the neuronal resting membrane potential, the spike frequency adaptation of neuronal firing, and presynaptic release. Impairment in their function leads to network instability even when lost exclusively in inhibitory neurons (Soh et al., 2018).

A significant percentage of childhood epilepsies are associated with KCNQ2 mutations (Lee et al., 2019). Human genetic studies identify de novo mutations in KCNQ2 as the third most robust link to epileptic encephalopathy (EE) (Zhao et al., 2020). Whether primary or secondary EEs, seizure activity worsens clinical outcomes and alters normal neurodevelopment (von Deimling, Helbig and Marsh, 2017).

Pediatric epilepsies affect about 1 in 200 children (Waaler et al., 2000) driving cognitive, behavioral and neurological deficits (Simkin and Kiskinis, 2018). In the case of specific pathogenic KCNQ2 mutations, despite most individuals eventually becoming seizure-free, developmental delays are experienced by the majority of patients (Kato et al., 2013). Targeting Kv7 channels offers a genetically validated target against epilepsy with a differentiated mode of action amongst anti-epileptics (Gunthorpe, Large and Sankar, 2012). Kv7.2 enhancers show the potential to transform neurodevelopmental trajectories by treating the neural network instability responsible for EEs (Kessi et al., 2020).

The connection between epilepsy and autism is robust (Srivastava and Sahin, 2017) and derives from the convergent phenotypes driven by a multitude of small genetic contributors, in combination with environmental factors. KCNQ2 is one of the top 5 ion channels associated with Autism Spectrum Disorder (ASD) and one of the top 30 of all de novo mutations known in ASD (Zhao et al., 2020).

Another defining feature of ASD, Atypical Sensory Processing (ASP) (Thye et al., 2018), is also driven by convergent genetics as seen in co-twin-control studies (Neufeld et al., 2021). The biology responsible for increased sensory sensitivity has been studied in preclinical models. There, multi-sensory neuronal hyper-excitability emerges regardless of the genetic manipulation that originally drives pathological neurodevelopment. Some genes whose manipulation leads to sensory sensitivity include CNTNAP2 (Penagarikano et al., 2011), SHANK3 (Holder and Quach, 2016) and GABRB3 (Tanaka et al., 2012). Kv7.2 enhancers show the potential to correct neurodevelopmental trajectories in ASD by normalizing network stability, neural information processing, and sensory abnormalities, ultimately responsible for atypical social and repetitive behaviors in ASD. It is also interesting that KCNQ2 knock-out mice show repetitive behaviors and aberrant exploratory and social behaviors (Kim et al., 2019).

Kv7.2 enhancers also showed promise in syndromic neurodevelopmental disorders in part because of the prevalence and impact of epilepsies (Budisteanu et al., 2020). For example, epilepsy is prevalent (>80%) in Angleman syndrome, mostly starting before 3 years of age (Fiumara et al., 2010).

Another neurodevelopmental disorder, Dup15q syndrome (Dup15q), is caused by the partial duplication of Chromosome 15 that confers a considerable risk for autism spectrum disorder, epilepsy, and intellectual disability. Dup15q patient-derived induced pluripotent cells show KCNQ2 anomalies, and Retigabine, a pan-Kv7 channel opener, partially corrects their phenotype (Fink et al., 2018). Epilepsies are central to Dup15q, with Kv7.2 enhancers showing potential to transform this neurodevelopmental disorder.

In Fragile X syndrome, about 15% of individuals experience epilepsy (Berry-Kravis, 2002) together with abnormal sensory processing (McCullagh et al., 2020). KCNQ2 (Kv7.2 gene) is downregulated in the absence of Fragile X Mental Retardation Protein (FMRP) in rodent models (Zhang et al., 2018). Therefore, Kv7.2 enhancers could positively impact Fragile X by acting on both epilepsies and sensory processing.

Infantile epilepsies are associated with intellectual disabilities, and KCNQ2 de novo mutations are significantly associated with intellectual disability (Zhao et al., 2020). Kv7.2 enhancement may address the underlying biology that exacerbates the disability.

For all these neurodevelopmental disorders, early diagnosis and the identification of the correct antiepileptic treatment is at the core of the strategies aiming at normalizing neurodevelopmental trajectories.

Within behavioral disorders, Kv7.2 enhancers showed promise in attention-deficit hyperactivity disorder (ADHD) as well as major depressive disorder (MDD, depression). Some patients with KCNQ2 mutations and mild epilepsy phenotype, show cognitive delay and ADHD (Lee et al., 2019). Kv7.2 enhancers were suggested to treat the neural network instability and the behavioral impulsivity linked to ADHD. In the MDD space, Retigabine (Kv7 opener) showed antidepressant efficacy in patients by acting on the brain's reward centers (Tan et al., 2018). The significant reduction in depressive symptoms observed with retigabine places Kv7.2 enhancers as therapeutic candidates in MDD.

The therapeutic potential of Kv7.2 enhancers in pain sensitivity is supported by the localization of Kv7.2 channels in dorsal root ganglia and their established role in pain perception (Brown and Passmore, 2009). Non-selective Kv7.2 enhancers showed efficacy in reducing the excitability of human peripheral axons (Lang et al., 2008). Retigabine has already shown some efficacy in preclinical pain models (Korsgaard et al., 2005; Xu et al., 2010; Wu et al., 2017). Retigabine also shows efficacy in controlling spreading depression, a wave of cellular depolarization associated with migraines (Aiba and Noebels, 2021).

Within sensory abnormalities, aberrant plasticity of KCNQ2 channels is strongly linked to the induction of tinnitus (Li, Choi and Tzounopoulos, 2013). This link is based on the localization of Kv7.2 channels in the cochlea (Jin et al., 2009) and how cochlear damage depends on neuronal excitability driven by the closure of Kv7.2 channels (Liu, Glowatzki and Fuchs, 2015). Retigabine prevents the development of tinnitus in preclinical models (Li, Choi and Tzounopoulos, 2013). As evidence in support of how KCNQ2 pathologies are connected between indications, it is interesting to find that tinnitus and hyperacusis are more prevalent in ASD than in the general population (Danesh et al., 2015).

In neurodegenerative diseases, dysregulated K+ homeostasis in chronic neuro-inflammatory conditions is central to disease progression. For example, in amyotrophic lateral sclerosis (ALS), a fatal neurodegenerative disease of the motor nervous system (Hardiman et al., 2017), diverse genetics converge onto motorneuron excitotoxicity (Kanai et al., 2006; Pasinelli and Brown, 2006) and specifically axonal hyperexcitability predicts survival (Kanai et al., 2012). Patient-derived motor neurons show membrane hyperexcitability and the tool compound Retigabine (pan-Kv7 enhancer) rescues phenotype (Wainger et al., 2014). The motorneuron hyperexcitability was found early in presymptomatic in vivo systems (Kuo et al., 2004) where it is a contributor to disease progression. Recently, clinical trials in ALS with Retigabine showed efficacy on functional biomarkers of ALS (Wainger et al., 2021) and preclinically protects against peripheral neuropathy (Nodera et al., 2011).

In Alzheimer's disease (AD), neuronal hyperexcitability and network instability (Frere and Slutsky, 2018) are early features of both IPSC models of sporadic AD (Ghatak et al., 2019), and genetic in vivo models (Palop et al., 2007; Kazim et al., 2017; Styr and Slutsky, 2018). Network instability worsens proteinopathy (Dolev et al., 2013; Frere and Slutsky, 2018) with consequences for patients (Vossel et al., 2013; Lam et al., 2017).

Because motor neuron and cortical neuron degeneration can be meaningfully slowed down by reducing aberrant neuronal activity, Kv7.2 enhancement could be an effective way to stop such aberrant activity, changing the neurodegenerative trajectory of the disease.

Therefore, enhancing the activity of Kv7.2 is a promising strategy for the treatment or prevention of diseases associated with Kv7.2. These include neurodevelopmental disorders like autism and Fragile X, epilepsy, intellectual disability, depression, attention deficit hyperactivity disorder, motor neuron excitability, pain, migraine, and sensory processing disorders.

WO2020/163268 relates to pyridine urea derivatives as KCNQ potentiators.

U.S. Pat. No. 5,384,330 relates to pharmacologically active 1,2,4-triaminobenzene derivatives modulating potassium ion channels Kv7.2-Kv7.5 (KCNQ2-KCNQ5) for the treatment of drug-resistant epilepsy.

The compounds showed tolerability issues and other side effects.

To date, no agents acting on Kv7.2 are approved for the treatment of any of the diseases, disorders, or disabilities described herein, and thus there remains a need for modulators of Kv7.2 which provide a therapeutic benefit. Further, it would be beneficial to have modulators of Kv7.2 which are highly selective over other Kv7 channels. There is a need for Kv7.2 modulators which provide for a combination of favorable pharmacological properties, such as for example potency, selectivity, and metabolic stability.

It is, therefore, an object of this invention to provide selective Kv7.2 enhancers with favorable pharmacological properties useful as Kv7.2 enhancers (or positive modulators) for the therapeutic and/or prophylactic treatment of disorders, diseases, or disabilities associated with Kv7.2.

### REFERENCES

Aiba, I. and Noebels, J. L. (2021) “Kcnq2/Kv7.2 controls the threshold and bihemispheric symmetry of cortical spreading depolarization,” Brain: a journal of neurology. doi: 10.1093/brain/awab141.
Berry-Kravis, E. (2002) “Epilepsy in fragile X syndrome,” Developmental medicine and child neurology. Wiley Online Library, 44(11), pp. 724-728.
Brown, D. A. and Passmore, G. M. (2009) “Neural KCNQ (Kv7) channels,” British journal of pharmacology, 156(8), pp. 1185-1195.
Budisteanu, M. et al. (2020) “Treatment of Epilepsy Associated with Common Chromosomal Developmental Diseases,” Open life sciences. Walter de Gruyter GmbH, 15(1), pp. 21-29.
Danesh, A. A. et al. (2015) “Tinnitus and hyperacusis in autism spectrum disorders with emphasis on high functioning individuals diagnosed with Asperger's Syndrome,” International journal of pediatric otorhinolaryngology, 79(10), pp. 1683-1688.
von Deimling, M., Helbig, I. and Marsh, E. D. (2017) “Epileptic Encephalopathies-Clinical Syndromes and Pathophysiological Concepts,” Current neurology and neuroscience reports, 17(2), p. 10.
Dolev, I. et al. (2013) “Spike bursts increase amyloid-β 40/42 ratio by inducing a presenilin-1 conformational change,” Nature neuroscience, 16(5), pp. 587-595.
Fink, J. J. et al. (2018) “Hyperexcitable phenotypes in iPSC-derived neurons from patients with 15q11-q13 duplication syndrome, a genetic form of autism,” BioRxiv. biorxiv.org. Available at: https://www.biorxiv.org/content/10.1101/286336v1.abstract.
Fiumara, A. et al. (2010) “Epilepsy in patients with Angelman syndrome,” Italian Journal of Pediatrics, p. 31. doi: 10.1186/1824-7288-36-31.
Frere, S. and Slutsky, I. (2018) “Alzheimer's Disease: From Firing Instability to Homeostasis Network Collapse,” Neuron, 97(1), pp. 32-58.
Ghatak, S. et al. (2019) “Mechanisms of hyperexcitability in Alzheimer's disease hiPSC-derived neurons and cerebral organoids vs isogenic controls,” eLife, 8. doi: 10.7554/eLife.50333.
Gunthorpe, M. J., Large, C. H. and Sankar, R. (2012) “The mechanism of action of retigabine (ezogabine), a first-in-class K+ channel opener for the treatment of epilepsy,” Epilepsia, 53(3), pp. 412-424.
Hardiman, O. et al. (2017) “Amyotrophic lateral sclerosis,” Nature reviews. Disease primers, 3, p. 17071.
Holder, J. L., Jr and Quach, M. M. (2016) “The spectrum of epilepsy and electroencephalographic abnormalities due to SHANK3 loss-of-function mutations,” Epilepsia, 57(10), pp. 1651-1659.
Jin, Z. et al. (2009) “Expression and localization of K channels KCNQ2 and KCNQ3 in the mammalian cochlea,” Audiology & neuro-otology, 14(2), pp. 98-105.
Kanai, K. et al. (2006) “Altered axonal excitability properties in amyotrophic lateral sclerosis: impaired potassium channel function related to disease stage,” Brain: a journal of neurology, 129 (Pt 4), pp. 953-962.
Kanai, K. et al. (2012) “Motor axonal excitability properties are strong predictors for survival in amyotrophic lateral sclerosis,” Journal of neurology, neurosurgery, andpsychiatry, 83(7), pp. 734-738.
Kato, M. et al. (2013) “Clinical spectrum of early onset epileptic encephalopathies caused by KCNQ2 mutation,” Epilepsia, 54(7), pp. 1282-1287.
Kazim, S. F. et al. (2017) “Early-Onset Network Hyperexcitability in Presymptomatic Alzheimer's Disease Transgenic Mice Is Suppressed by Passive Immunization with Anti-Human APP/Aβ Antibody and by mGluR5 Blockade,” Frontiers in aging neuroscience, 9, p. 71.
Kessi, M. et al. (2020) “Intellectual Disability and Potassium Channelopathies: A Systematic Review,” Frontiers in genetics, 11, p. 614.
Kim, E. C. et al. (2019) “Heterozygous loss of epilepsy gene KCNQ2 alters social, repetitive, and exploratory behaviors,” Genes, Brain and Behavior. doi: 10.1111/gbb.12599.
Korsgaard, M. P. G. et al. (2005) “Anxiolytic effects of Maxipost (BMS-204352) and retigabine via activation of neuronal Kv7 channels,” The Journal of pharmacology and experimental therapeutics, 314(1), pp. 282-292.
Kuo, J. J. et al. (2004) “Hyperexcitability of cultured spinal motoneurons from presymptomatic ALS mice,” Journal of neurophysiology. physiology.org, 91(1), pp. 571-575.
Lam, A. D. et al. (2017) “Silent hippocampal seizures and spikes identified by foramen ovale electrodes in Alzheimer's disease,” Nature medicine, 23(6), pp. 678-680.
Lang, P. M. et al. (2008) “Retigabine reduces the excitability of unmyelinated peripheral human axons,” Neuropharmacology, 54(8), pp. 1271-1278.
Lee, I.-C. et al. (2019) “KCNQ2 mutations in childhood nonlesional epilepsy: Variable phenotypes and a novel mutation in a case series,” Molecular genetics & genomic medicine, 7(7), p. e00816.
Li, S., Choi, V. and Tzounopoulos, T. (2013) “Pathogenic plasticity of Kv7.2/3 channel activity is essential for the induction of tinnitus,” Proceedings of the National Academy of Sciences of the United States of America. National Academy of Sciences, 110(24), pp. 9980-9985.
Liu, C., Glowatzki, E. and Fuchs, P. A. (2015) “Unmyelinated type II afferent neurons report cochlear damage,” Proceedings of the National Academy of Sciences of the United States of America, 112(47), pp. 14723-14727.
McCullagh, E. A. et al. (2020) “Mechanisms underlying auditory processing deficits in Fragile X syndrome,” FASEB journal: official publication of the Federation of American Societies for Experimental Biology. doi: 10.1096/fj.201902435R.
Neufeld, J. et al. (2021) “A co-twin-control study of altered sensory processing in autism,” Autism: the international journal of research and practice, 25(5), pp. 1422-1432.
Ng, F. L et al. (2011). Expression and function of the K+ channel KCNQ genes in human arteries. British Journal of Pharmacology, 162(1), 42-53.
Nodera, H. et al. (2011) “Neuroprotective effects of Kv7 channel agonist, retigabine, for cisplatin-induced peripheral neuropathy,” Neuroscience letters, 505(3), pp. 223-227.
Palop, J. J. et al. (2007) “Aberrant excitatory neuronal activity and compensatory remodeling of inhibitory hippocampal circuits in mouse models of Alzheimer's disease,” Neuron, 55(5), pp. 697-711.
Pasinelli, P. and Brown, R. H. (2006) “Molecular biology of amyotrophic lateral sclerosis: insights from genetics,” Nature reviews. Neuroscience, 7(9), pp. 710-723.
Penagarikano, O. et al. (2011) “Absence of CNTNAP2 leads to epilepsy, neuronal migration abnormalities, and core autism-related deficits,” Cell, 147(1), pp. 235-246.
Simkin, D. and Kiskinis, E. (2018) “Modeling Pediatric Epilepsy Through iPSC-Based Technologies,” Epilepsy currents American Epilepsy Society, 18(4), pp. 240-245.
Soh, H. et al. (2018) “Deletion of KCNQ2/3 potassium channels from PV+ interneurons leads to homeostatic potentiation of excitatory transmission,” eLife, 7. doi: 10.7554/eLife.38617.
Srivastava, S. and Sahin, M. (2017) “Autism spectrum disorder and epileptic encephalopathy: common causes, many questions,” Journal of neurodevelopmental disorders, 9, p. 23.
Styr, B. and Slutsky, I. (2018) “Imbalance between firing homeostasis and synaptic plasticity drives early-phase Alzheimer's disease,” Nature neuroscience, 21(4), pp. 463-473.
Tan, A. et al. (2018) “Effects of the KCNQ channel opener ezogabine on functional connectivity of the ventral striatum and clinical symptoms in patients with major depressive disorder,” Molecular psychiatry. doi: 10.1038/s41380-018-0283-2.
Tanaka, M. et al. (2012) “GABRB3, Epilepsy, and Neurodevelopment,” in Noebels, J. L. et al. (eds.) Jasper's Basic Mechanisms of the Epilepsies. Bethesda (MD): National Center for Biotechnology Information (US).
Thye, M. D. et al. (2018) “The impact of atypical sensory processing on social impairments in autism spectrum disorder,” Developmental cognitive neuroscience. Elsevier, 29, pp. 151-167.
Vossel, K. A. et al. (2013) “Seizures and epileptiform activity in the early stages of Alzheimer disease,” JAMA neurology, 70(9), pp. 1158-1166.
Waaler, P. E. et al. (2000) “Prevalence, classification, and severity of epilepsy in children in western Norway,” Epilepsia, 41(7), pp. 802-810.
Wainger, B. J. et al. (2014) “Intrinsic membrane hyperexcitability of amyotrophic lateral sclerosis patient-derived motor neurons,” Cell reports, 7(1), pp. 1-11.
Wainger, B. J. et al. (2021) “Effect of Ezogabine on Cortical and Spinal Motor Neuron Excitability in Amyotrophic Lateral Sclerosis: A Randomized Clinical Trial,” JAMA neurology, 78(2), pp. 186-196.
Wang, H. S. et al. (1998) “KCNQ2 and KCNQ3 potassium channel subunits: molecular correlates of the M-channel,” Science. American Association for the Advancement of Science, 282(5395), pp. 1890-1893.
Wu, Z. et al. (2017) “Activation of KCNQ Channels Suppresses Spontaneous Activity in Dorsal Root Ganglion Neurons and Reduces Chronic Pain after Spinal Cord Injury,” Journal of neurotrauma, 34(6), pp. 1260-1270.
Xu, W. et al. (2010) “Activation of voltage-gated KCNQ/Kv7 channels by anticonvulsant retigabine attenuates mechanical allodynia of inflammatory temporomandibular joint in rats,” Molecular pain, 6, p. 49.
Zhang, F. et al. (2018) “Fragile X mental retardation protein modulates the stability of its m6A-marked messenger RNA targets,” Human molecular genetics. Oxford Academic, 27(22), pp. 3936-3950.
Zhao, G. et al. (2020) “Gene4Denovo: an integrated database and analytic platform for de novo mutations in humans,” Nucleic acids research. Oxford Academic, 48(D1), pp. D913-D926.

### SUMMARY OF THE INVENTION

In some aspects, provided herein is a compound of formula (I), or a solvate or a pharmaceutically acceptable salt thereof:

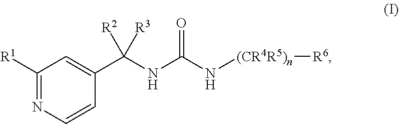

wherein R¹, R², R³, R⁴, R⁵, n and R⁶ are as defined herein.
In a further aspect, the present invention provides a pharmaceutical composition comprising a compounds of formula (I), or a solvate or a pharmaceutically acceptable salt thereof.

In a further aspect, the present invention provides a compound of formula (I), or a solvate or a pharmaceutically acceptable salt thereof, or a pharmaceutical composition comprising the same, for use as therapeutically active substance.

In a further aspect, the present invention provides a compound of formula (I), or a solvate or a pharmaceutically acceptable salt thereof, or a pharmaceutical composition comprising the same, for use in the therapeutic and/or prophylactic treatment of a disorder, disease, or disability associated with Kv7.2.

In a further aspect, the present invention provides the use of a compound of formula (I), or a solvate or a pharmaceutically acceptable salt thereof, or a pharmaceutical composition comprising the same, in the therapeutic and/or prophylactic treatment of a disorder, disease, or disability associated with Kv7.2.

In a further aspect, the present invention provides the use of a compound of formula (I), or a solvate or a pharmaceutically acceptable salt thereof, or a pharmaceutical composition comprising the same, for the manufacture of a medicament for the therapeutic and/or prophylactic treatment of a disorder, disease, or disability associated with Kv7.2.

In a further aspect, the present invention provides a method for the therapeutic and/or prophylactic treatment of a disorder, disease, or disability associated with Kv7.2, which method comprises administering a therapeutically effective amount of a compound of formula (I), or a solvate or a pharmaceutically acceptable salt thereof, or a pharmaceutical composition comprising the same.

In a further aspect, the present invention provides a kit for use in the therapeutic and/or prophylactic treatment of a disorder, disease, or disability associated with Kv7.2, comprising:
a) a compound of formula (I), or a solvate or a pharmaceutically acceptable salt thereof, or a pharmaceutical composition or a pharmaceutical composition for use comprising the same; and
b) instructions for use.

In a further aspect, the present invention provides the invention as hereinbefore described.

The compounds of formula (I), or a solvate or a pharmaceutically acceptable salts thereof, as described herein, provide for a combination of favorable pharmacological properties, such as for example potency, selectivity, and metabolic stability. A reasonable metabolic stability is important to ensure a suitable pharmacological half life, which is best achieved with compounds that have a human liver microsomal clearance below 20 uL/min/mg. Selectivity within the Kv7 family is desirable to avoid actions on tissues without therapeutic potential for the indications described in this invention. For example, actions on Kv7.4 and Kv7.5 in skeletal and smooth muscle impact the function of human arteries, where KCNQ2 expression is minimal or undetected in these tissues (Ng et al 2011).

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments explicitly disclosed herein. Any embodiment described in this application can be combined with any other embodiment. The invention extends to any novel one, or any novel combination, of the features disclosed in this specification (including any accompanying claims, and abstract), or to any novel one, or any novel combination, of the any embodiment, or any steps of any method or process so disclosed.

“Administer”, “administered”, or “administering” when used for the therapeutic and/or prophylactic treatment of disorders, diseases, or disabilities as described herein means the giving of a compound of this invention to a patient or subject by any method, e.g. by infusion, inhalation, injection, paste, suppository, or tablet, etc.

As used herein, the terms “including”, “containing”, and “comprising” are used in their open, non-limiting sense.

The articles “a” and “an” as used in this disclosure may refer to one or more than one (e.g., to at least one) of the grammatical object of the article. By way of example, “an element” may mean one element or more than one element.

As described herein, compounds of the present disclosure may be “unsubstituted” or “substituted” with one or more substituents (e.g., 1, 2, 3, 4, or 5), such as those illustrated generally herein, or as exemplified by particular classes, subclasses, and species of the present disclosure. In general, the term “substituted” refers to the replacement of a hydrogen atom in a given structure with a specified substituent. In some embodiments, more than one hydrogen atom is replaced with a specified substituent (e.g. when two hydrogen atoms are replaced with one oxo substituent). Combinations of substituents envisioned by the present disclosure are typically those that result in the formation of stable or chemically feasible compounds. In one embodiment, an optionally substituted group has one substituent. In another embodiment, an optionally substituted group has two substituents. In another embodiment, an optionally substituted group has three substituents. In another embodiment, an optionally substituted group has substituents as described herein.

As used herein, the term “unsubstituted” may mean that the specified group bears no substituents beyond the moiety recited (e.g., where valency is satisfied by hydrogen).

The terms “effective amount” or “therapeutically effective amount” refers to an amount of a compound of formula (I), or a solvate or a pharmaceutically acceptable salt thereof, or a pharmaceutical composition of the afore-mentioned, being sufficient to produce a desired therapeutic outcome, such as reducing the severity of duration of, stabilizing the severity of, or eliminating one or more signs, symptoms or causes of a disease, disorder, or disability. For therapeutic use, beneficial or desired results may include, for example, decreasing one or more symptoms resulting from the disease, disorder, or disability (biochemical, histologic and/or behavioral), including its complications and intermediate pathological phenotypes presenting during development of the disease, disorder, or disability, increasing the quality of life of those suffering from the disease, disorder, or disability, decreasing the dose of other medications required to treat the disease, disorder, or disability, enhancing effect of another medication, delaying the progression of the disease, disorder, or disability and/or prolonging survival of patients.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins, and the like.

The term “excipient” or “pharmaceutical excipients” as used herein refers to any pharmaceutically acceptable excipient that may be used in the production of a drug or pharmaceutical composition, such as a tablet containing a compound as described herein (or tautomer or pharmaceutically acceptable salt) as an active ingredient. Various substances may be embraced by the term excipient, including without limitation any substance used as a diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Disintegrant refers to excipients that expand and dissolve when wet causing the tablet to break apart in the body and release the active ingredient for absorption. Examples include cross-linked polymers like crospovidone, croscarmellose sodium, etc. and modified starches like sodium starch glycolate. Filler refers to excipients that fill out the size of a tablet by increasing the bulk volume. Fillers make it possible for the final product to have the proper volume for patient handling. Examples of fillers are plant cellulose, lactose, starch, mannitol, etc. Specific examples are lactose monohydrate like Pharmatose 200M, microcrystalline cellulose (MCC) like Avicel PH101, or Avicel PH102 and spray dried lactose like Fast Flo 316™. Binders refers to excipients that hold the ingredients in a tablet together. Binders ensure that tablets and granules can be formed with required mechanical strength. Examples of binders are, polyvinlypyrrolidon (PV), hydroxypropyl methylcellulose (HPMC), hydroxypropylcellulose (HPC), cellulose, sugar alcohols like sorbitol, proteins like gelatin and polymers like PVP, e.g. copovidone (PVP/VA 64), PEG, etc. Lubricants refer to excipients that prevent ingredients from clumping together and from sticking to the tablet punches or capsule filling machine. Lubricants also ensure that tablet formation and ejection can occur with low fraction between active ingredient and wall. Examples of lubricants are minerals like talc or silica and fats like stearin, magnesium stearate, etc. Coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch dc, sucrose, etc. Creams or lotions include, e.g., maltodextrin, carrageenans, etc. Materials for chewable tablets include, e.g. dextrose, fructose dc, lactose (monohydrate, optionally in combination with aspartame or cellulose), etc. Suspending/gelling agents include, e.g., carrageenan, sodium starch glycolate, xanthan gum, etc. Sweeteners include, e.g., aspartame, dextrose, fructose dc, sorbitol, sucrose dc, etc. Wet granulation agents include, e.g., calcium carbonate, maltodextrin, microcrystalline cellulose, etc. In some cases, the term “excipient” encompasses pharmaceutically acceptable carriers. The skilled person knows suitable pharmaceutical compositions to be used in the treatment of patients and how to produce them.

The term a “patient” or “subject” may encompass both mammals and non-mammals. Examples of mammals may include, but are not limited to, any member of the class Mammalia: humans; nonhuman primates such as chimpanzees, monkeys, baboons, or rhesus monkeys, as well as other apes and monkey species; farm animals such as cattle, horses, sheep, goats, and swine; companion animals such as rabbits, dogs, and cats; laboratory animals including rodents, such as rats, mice and guinea pigs; and the like. Examples of non-mammals include, but are not limited to, birds, fish, and the like. “Patient” or “subject” may include both human and animals. In some preferred embodiments, the “patient” or “subject” is a human.

As used herein, the terms “treat” or “treatment” are meant to indicate a postponement of development of one or more disease(s), disorder(s), or disability(ies); preventing the development of one or more disease(s), disorder(s), or disability(ies); and/or reducing severity of one or more symptoms of a disease, disorder, or disability that will or are expected to develop. Thus, these terms may include ameliorating one or more existing disease, disorder, or disability symptoms; preventing one or more additional symptoms; ameliorating or preventing the underlying causes of one or more symptoms; inhibiting the diseases, disorder, or disability, e.g., arresting the development of the diseases, disorder, or disability; relieving the diseases, disorder, or disability; causing regression of the diseases, disorder, or disability; relieving a symptom caused by the diseases, disorder, or disability; or stopping or alleviating the symptoms of the diseases, disorder, or disability.

Compounds of the invention and disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water.

The term “prophylaxis” as used herein includes: preventing or delaying the appearance of clinical symptoms of diseases, disorder, or disability developing in a patient or subject, especially a human, that may be afflicted with or predisposed to the disease, disorder, or disability as described herein, but does not yet experience or display clinical or subclinical symptoms of the disease, disorder, or disability.

As used herein, the term “about,” when referring to a value is meant to encompass variations of, for example, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller ranges is also encompassed within the invention, subject to any specifically excluded limit in the stated range. Where the stated range includes one or both of the limits, ranges excluding either or both of those included limits are also included in the invention.

Numerical ranges, as used herein, may include sequential integers. For example, a range expressed as “from 0 to 5” would include 0, 1, 2, 3, 4, and 5.

A “metabolite” is a product produced through metabolism in the body of a specified compound or salt thereof. Metabolites of a compound may be identified using routine techniques known in the art and their activities determined using tests such as those described herein. Such products may result e.g. from the oxidation, reduction, hydrolysis, amidation, deamidation, esterification, deesterification, enzymatic cleavage, and the like, of the administered compound.

Accordingly, the invention includes metabolites of compounds of the invention, including compounds produced by a process comprising contacting a compound of this invention with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

The terms “compound of this invention,” and “compounds of the present invention” “compounds of the invention” and “compounds of formula (I)” include compounds of formula (I), compounds selected from any of formulae (II), (III), (III*), (IV), (V), (VI), (VII), etc., compounds from Tables 1, 2, 3, A, B, etc., stereoisomers, geometric isomers, solvates, pharmaceutically acceptable salts, tautomers, metabolites, prodrugs, polymorphs; and mixtures thereof.

The symbols “*” at the end of a bond or “- - -” drawn through a bond each refer to the point of attachment of a functional group or other chemical moiety to the rest of the molecule of which it is a part. Thus, for example:

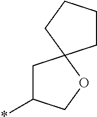 [SMILES: *C1COC2(CCCC2)C1]

means that the substituent is attached to the rest of the molecule as shown

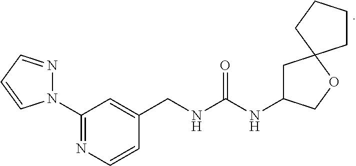 [SMILES: O=C(NCc1ccnc(-n2cccn2)c1)NC1COC2(CCCC2)C1]

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent. “Optionally substituted” means that a compound can be unsubstituted or substituted as defined herein. “Optionally substituted” means that a given substituent can be unsubstituted or further be substituted with certain substituents as defined herein and listed in the different embodiments. E.g. as a non-limiting example, the wording R⁶ can be cyclobutyl optionally substituted with one or two C₁₋₆alkyl means that R⁶ comprises unsubstituted cyclobutyl or cyclobutyl substituted with one or two C₁₋₆alkyl.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen. In an addition, e.g. in a compound in which e.g. R² and R³ are independently selected from hydrogen and hydroxyC₁₋₆alkyl, both R² and R³ can be hydrogen, or both can be hydroxyC₁₋₆alkyl, or one of R² and R³ can be hydrogen and the other one hydroxyC₁₋₆alkyl.

In this application the units ul, uMol, C etc. mean μl, μMol, ° C. etc.

The term “EC₅₀” in this application is defined as: the agonistic effect of a compound can be determined by testing the compound in an in vitro assay as described herein, whereby the effect of the compound is measured across a range of compound concentrations. The resulting data is plotted as a concentration response curve, which typically follows a sigmoidal function, whereby the concentration of the compound is plotted on the x axis and the response (agonistic effect) is plotted on the y axis. The term “EC₅₀” is the “half maximal effective concentration” and denotes the concentration of a particular compound required to obtain 50% of the maximum response (Eₘₐₓ) which is observed for that compound in the given in vitro assay.

A compound of this invention may exist in one or more stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt may exist in tautomeric forms other than that shown in the formula and these are also included within the scope of the subject matter disclosed herein. It is to be understood that the subject matter disclosed herein includes combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

A compound of this invention can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “chiral” refers to molecules which have the property of non-superimposability of the mirror image partner, while the term “achiral” refers to molecules which are superimposable on their mirror image partner. Chiral separation of a racemate to its enantiomeric components may be performed to separate the eutomer and the distomer.

The term “stereoisomers” refers to compounds, which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Any organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process.

The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

It should be understood that individual enantiomers and diastereomers are included in the tables below by compound name, and their corresponding structures can be readily determined therefrom. In some instances, the enantiomers or diastereomers are identified by their respective properties, for example, retention times on a chiral HPLC or their biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

In some embodiments of the invention only one of the possible enantiomers are used. In other embodiments mixtures of the possible enantiomers having different percentages for each component are used.

In some embodiments, the compounds of this invention are isotopically-labeled by having one or more atoms therein replaced by an atom having a different atomic mass or mass number. Such isotopically-labeled (e.g., radiolabeled) compounds of formula (I), or a solvate or a pharmaceutically acceptable salts thereof, are considered to be within the scope of this disclosure. Examples of isotopes that can be incorporated into the compounds of formula (I), or a solvate or a pharmaceutically acceptable salts thereof, include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), or a solvate or a pharmaceutically acceptable salts thereof, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of this invention can be enriched with 1, 2, 5, 10, 25, 50, 75, 90, 95, or 99 percent of a given isotope.

Substitution with positron emitting isotopes, such as ³H, ¹¹C, ¹⁸F, ¹⁵O and ¹³N, can be useful in Positron Emission Topography (PET) studies for examining substrate receptor occupancy. Isotopically-labeled compounds of this invention can generally be prepared by conventional techniques known to those skilled in the art or by processes analogous to those described in the Examples as set out below using an appropriate isotopically-labeled reagent in place of the non-labeled reagent previously employed.

In one embodiment, the present invention provides pharmaceutically acceptable salts of the compounds of this invention especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I), or solvates or pharmaceutically acceptable salts thereof, as described herein (i.e., as “free bases” or “free acids”, respectively).

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

The term “alkyl” refers to a mono- or multivalent, e.g., a mono- or bivalent, linear or branched saturated hydrocarbon group of 1 to 6 carbon atoms (“C₁₋₆alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of methyl (CH₃—), ethyl (CH₃CH₂—), n-propyl (CH₃CH₂CH₂—), 2-propyl (isopropyl, (CH₃)₂CH₂—), n-butyl (CH₃CH₂CH₂CH₂—), iso-butyl ((CH₃)₂CH₂CH₂—), sec-butyl (CH₃CH(CH₃)CH₂—), and tert-butyl ((CH₃)₃C—). Preferred, yet non-limiting, examples of alkyl are tert-butyl ((CH₃)₃C—) or methyl (CH₃—). A preferred, yet non-limiting, example of alkyl is methyl (CH₃—). Another preferred, yet non-limiting, example of alkyl is tert-butyl ((CH₃)₃C—).

The term “alkoxy” refers to an alkyl group, as previously defined, attached to the parent molecular moiety via an oxygen atom. The alkoxy group contains 1 to 6 carbon atoms (“C₁₋₆alkoxy”), e.g. 1, 2, 3, 4, 5, or 6 carbon atoms. In other embodiments, the alkoxy group contains 1 to 4 carbon atoms. In still other embodiments, the alkoxy group contains 1 to 3 carbon atoms. Some non-limiting examples of alkoxy groups include CH₃O— (methoxy), CH₃CH₂O— (ethoxy), CH₃CH₂CH₂O— (n-propoxy), and (CH₃)₃CO— (tert-butoxy). A particularly preferred, yet non-limiting, example of alkoxy is methoxy (CH₃O—).

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “7-11 membered spirocylic cyloalkyl” as used herein refers to a bicyclic spirocyclic cycloakyl comprising 7, 8, 9, 10, or 11 C-atoms with two saturated non-aromatic rings. In some embodiments, the ring directly attached to the molecule is not a 4 membered ring. In some examples, the 7-10 membered spirocyclic cycloalkyl is selected from spiro[3.3]heptanyl, spiro[2.3]hexanyl, spiro[3.4]octanyl optionally substituted as described herein.

The term “7-11 membered oxo-spiro-heterocycloalkyl” as used herein refers to bicyclic oxa-spiro-heteroccyloalkyls comprising 7, 8, 9, 10, or 11 ring atoms, comprising two saturated monocyclic rings, wherein one rings comprise one O-atom. In some embodiments, the O-atom is in the ring directly attached to the molecule. In some embodiments, the ring directly attached to the molecule is not a 4 membered ring. In some embodiments, the 7-11 membered oxa-spiro-heterocycloalkyl is selected from oxaspiroheptanyl, oxaspirooctanyl, oxaspirononanyl, oxaspirodecanyl, and oxaspiroundecanyl. In some preferred embodiments, the oxa-spiro-heterocycloalkyl comprises one O-atom to form a 9-10 membered oxa-spiro-heterocycloalkyl. Non-limiting examples of 9-10 membered oxa-spiro-heterocycloalkyl are selected from oxaspirononanyl and oxaspirodecanyl. All oxo-spiro-heterocycloalkyls can be substituted as described herein.

“Heteroaryl” as used herein refers to a 5 or 6 membered monocyclic, aromatic group comprising at least one ring heteroatom. In some embodiments, the heteroatom is independently selected from the group consisting of N-atoms, 0-atoms, and S-atoms. The number of ring atoms refer to the sum of carbon and heteroatoms in the one ring. In some embodiment, heteroaryl is a 5 or 6 membered monocyclic, aromatic group with two N-atoms. In some embodiments, heteroaryl is a 5 or 6 membered monocyclic aromatic group comprising one N-atom. In some embodiments, heteroaryl is a 5 or 6 membered monocyclic aromatic group comprising one N-atom and one O-atom. Examples of 5 membered heteroaryl groups include, but are not limited to, pyrrolyl, pyrazolyl, imidazolyl, oxazolyl, triazolyl, or furanyl. In some preferred embodiments, 5 membered heteroaryl is pyrazolyl, imidazolyl, or oxazolyl. In some more preferred embodiments, 5 membered heteroaryl is pyrazolyl or imidazolyl. Examples of 6 membered heteroaryl groups include, but are not limited to, pyrimidinyl, pyridinyl, pyrazinyl, or pyridazinyl. In some more preferred embodiments, 6 membered heteroaryl is pyrazinyl, pyridinyl or pyrimidinyl.

The term “C₃₋₅cycloalkylC₁₋₆alkoxy” as used herein refers to an alkoxy group, wherein at least one of the hydrogen atoms was replaced by a C₃₋₅cycloalkyl group. Preferably, “C₃₋₅cycloalkylC₁₋₆ alkoxy” refers to an alkoxy group wherein one, two, or three hydrogen atoms of the alkoxy group have been replaced by a C₃₋₅cycloalkyl group. Preferably, “C₃₋₅cycloalkylC₁₋₆alkoxy” refers to an alkoxy group wherein one hydrogen atom of the alkoxy group has been replaced by a C₃₋₅cycloalkyl group. Preferably, the alkoxy group comprises one C-atom. Preferred, yet not-limiting, examples are 2-cyclopropylmethoxy and 2-cyclobutylmethoxy.

The term “C₃₋₅heterocyclyloxy” as used herein refers to an alkoxy group, wherein a 3-5 membered heterocycloalkyl group is linked to an —O— to form an alkoxy group. Preferably, a yet not-limiting, example is oxetanyloxy (specifically oxetan-3-yloxy).

The term “cyano” refers to a —CN (nitrile) group.

The term “hydroxy” or “hydroxyl” refers to an OH group.

The term “haloalkyl” refers to a C₁₋₆alkyl group as described above, with one to six C-atoms, wherein at least one of the hydrogen atoms of the alkyl group has been replaced by one or more halogen atoms. Preferably, “haloalkyl” refers to an alkyl group wherein 1, 2 or 3 hydrogen atoms of the alkyl group have been replaced by a halogen atom, i.e. haloalkyl includes monohaloalkyl, dihaloalkyl, trihaloalkyl, perhaloalkyl and the like. Halogen atoms may be fluoro (F), chloro (Cl), or bromo (Br). Particularly preferred, yet non-limiting, examples of “halogen” are fluoro (F) and chloro (Cl). More preferably, haloalkyl is substituted with fluoro (F). Preferred, yet non-limiting, examples of haloalkyl are (CH₃)₂FC-(1-fluoro-isopropyl), CF₃CH₂-(2,2,2-trifluoroethyl), CH₃CF₂-(1,1-difluoroethyl), CF₃— (trifluoromethyl), CH₂F— (fluoromethyl), or CHF₂— (difluoromethyl). Particularly preferred are CH₃CF₂—, and CF₃—.

“Haloalkoxy”, as used herein, refers to a C₁₋₆alkoxy group as described above, wherein at least one of the hydrogen atoms has been replaced by halogen atoms. Preferably, “haloalkoxy” refers to an alkoxy group wherein 1, 2, or 3 hydrogen atoms of the alkyl group have been replaced by a halogen atom, i.e. haloalkoxy includes monohaloalkoxy, dihaloalkoxy, trihaloalkoxy, perhaloaloxy and the like. Halogen atoms may be fluoro (F), chloro (Cl) or bromo (Br). Particularly preferred, yet non-limiting, examples of “halogen” are fluoro (F) and chloro (Cl). More preferably, haloalkoxy is substituted with fluoro (F). Particularly preferred, yet non-limiting examples of haloalkoxy are FCH₂CHFCH₂O—, CHF₂O—, CH₂FO—, CF₃CH₂O—, CF₂HCH₂O—, CH₃CF₂CH₂O—, and CH₃CFHCH₂O—. Particularly preferred are CHF₂O—, CH₃CF₂CH₂O—, and CH₃CFHCH₂O—.

The term “hydroxyalkyl” refers to a C₁₋₆alkyl group as described above, with one to six C-atoms, wherein at least one of the hydrogen atoms of the alkyl group has been replaced by one or more hydroxy. Preferably, “hydroxyalkyl” refers to an alkyl group wherein 1, 2 or 3 hydrogen atoms of the alkyl group have been replaced by hydroxy, i.e. hydroxyalkyl includes monohydroxyalkyl, dihydroxalkyl, trihydroxyalkyl, perhydroxyalkyl and the like. More preferably, “hydroxyalkyl” refers to a C₁₋₆alkyl group wherein one hydrogen atom has been replaced by hydroxy. Particularly preferred, yet not limiting examples of hydroxyalkyl are HOCH₂— (hydroxymethyl), HOCH₂CH₂-(hydroxyethyl),

The term “mood disorder” as used herein relates to a mental health problem that primarily affects a person's emotional state. It is a disorder in which a person experiences long periods of extreme happiness, extreme sadness or both. Two of the most common mood disorders are depression and bipolar disorder.

The term “depression” as used herein relates to a mood disorder that causes a persistent feeling of sadness and loss of interest. It is also known as major depressive disorder (MDD).

The term “behavioral disorder” relates to disorders that involve a pattern of disruptive behaviors in children that last for at least 6 months and cause problems in school, at home and in social situations. Behavioral disorders involve a pattern of disruptive behaviors in children that last for at least 6 months and cause problems in school, at home and in social situations. The most important behavioral disorder is Attention deficit hyperactivity disorder” (ADHD).

The term “Attention deficit hyperactivity disorder” (ADHD) as used herein relates to a behavioral disorder characterized by inattention, or excessive activity and impulsivity. ADHD occurs more frequently in people with epilepsy than in the general population. Children with ADHD have an increased risk of seizures, with approximately 14% of children with ADHD developing seizures.

The term “developmental disorder” or “neurodevelopmental disorder” as used herein relates to a group of conditions caused by an impairment in physical, learning, language, or behavior areas. These conditions begin during the developmental period, may impact day-to-day functioning, and can last through a person's lifetime. Examples of neurodevelopment disorders include Autism Spectrum Disorder (“ASD”) and syndromic developmental disorders.

The term “Autism Spectrum Disorder (ASD)” as used herein relates to a developmental disorder of variable severity that is characterized by difficulty in social interaction and communication and by restricted or repetitive patterns of thought and behavior. ASD encompasses mostly idiopathic but also syndromic forms and is currently diagnosed according to the diagnostic and statistical manual version 5 (DSM V).

The term “syndromic developmental disorder” as used herein relates to a development disorder with a clinically defined pattern of somatic abnormalities and a neurobehavioral phenotype that may include ASD. The diagnosis is typically confirmed by targeted genetic testing. Examples for syndromic development disorders include Dup15q syndrome (Dup15q), Fragile X syndrome (FXS) and Angelman syndrome.

The term “Dup15q syndrome” or “Duq15q” as used herein relates to the common name for chromosome 15q11.2-q13.1 duplication syndrome. This is a syndromic development disorder, caused by the partial duplication of Chromosome 15, which confers a strong risk for autism spectrum disorder, epilepsy and intellectual disability.

The term “Fragile X syndrome” (FXS) as used herein relates to a genetic disorder characterized by mild-to-moderate intellectual disability. This disorder is typically caused by an expansion of the CGG triplet repeat within the FMR1 (fragile X mental retardation 1) gene on the X chromosome.

The term “Angelman syndrome” as used herein relates to a genetic disorder that mainly affects the nervous system due to a lack of function of part of chromosome 15 inherited from a person's mother. Characteristic features of this condition include delayed development, intellectual disability, severe speech impairment, and problems with movement and balance (ataxia). Most affected children also have recurrent seizures (epilepsy).

The term “Intellectual disability” (ID) used herein relates to a generalized neurodevelopmental disorder characterized by significantly impaired intellectual and adaptive functioning. It is defined by an IQ under 70, in addition to deficits in two or more adaptive behaviors that affect everyday, general living. ID is also known as a general learning disability and formerly known as mental retardation (MR).

The term “epilepsy” used herein relates to a neurological disorder marked by sudden recurrent episodes of sensory disturbance, loss of consciousness, or convulsions, associated with abnormal electrical activity in the brain. Examples of epilepsies include broad pediatric epilepsies, West syndrome, Ohtahara syndrome and epileptic encephalopathy.

The term “neurodegenerative diseases” used herein relates to diseases that are related to e progressive loss of structure or function of neurons, including the death of neurons. Examples of neurodegenerative diseases include, but are not limited to, Alzheimer's disease and motor neuron diseases.

The term “motor neuron disease” used herein relates to a group of rare neurodegenerative disorders that selectively affect motor neurons. Examples of motor neuron diseases include, but are not limited to, amyotrophic lateral sclerosis (ALS).

The term “pain” as used herein relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain.

The term “migraine” as used herein relates to a moderate to severe headache disorder, causing throbbing or pulsating pain for hours or days.

The term “Tinnitus” as used herein relates to a symptom characterized by the perception of sound when no corresponding external sound is present.

Any disease, disorder, or disability described herein also includes any state or condition related to such disease, disorder, or disability.

### Compounds of the Invention

All compound names of compound structures were generated using OpenEye Lexichem, Version 1.2.0, OpenEye Scientific Software, Santa Fe, NM, USA; www.eyesopen.com.

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiments described herein. Any embodiment described in this application can be combined with any other embodiment. For example, any embodiment herein relating to the compounds of this invention can be combined with any embodiment of pharmaceutical compostions, kits, medical use, or method of treatment. The invention extends to any novel one, or any novel combination, of the features disclosed in this specification (including any accompanying claims, abstract and drawings), or to any novel one, or any novel combination, of the steps of any method or process so disclosed.

The present invention relates to a compound of formula (I):

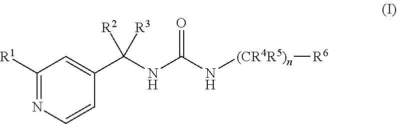

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
or a solvate or a pharmaceutically acceptable salt thereof.

In one embodiment, the present invention provides a compound of formula (I):

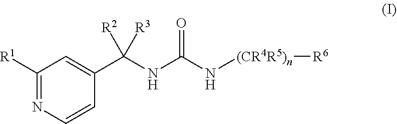

wherein
R¹ is selected from a 5-6 membered heteroaryl, haloC₁₋₆alkoxy, C₃₋₅cycyloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cycl yloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H, hydroxy, hydroxyC₁₋₆alkyl, or halogen;
R⁵ is H, hydroxy, hydroxyC₁₋₆alkyl, or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

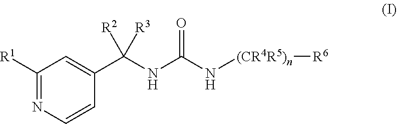

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cycloalkylalkoxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H, hydroxy, hydroxyC₁₋₆alkyl, or halogen;
R⁵ is H, hydroxy, hydroxyC₁₋₆alkyl, or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

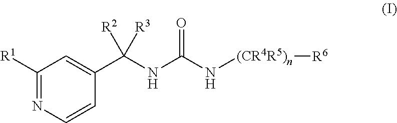

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H, hydroxy, hydroxyC₁₋₆alkyl, or halogen;
R⁵ is H, hydroxy, hydroxyC₁₋₆alkyl, or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

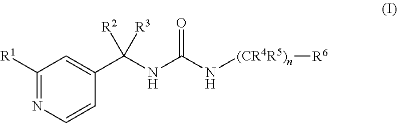

wherein
R¹ is selected from a 5-6 membered heteroaryl, haloC₁₋₆alkoxy, C₃₋₅cycyloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

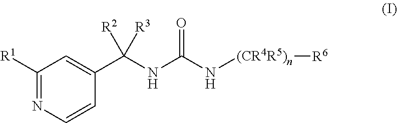

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cycloalkylalkoxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

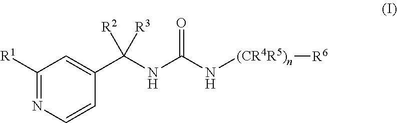

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, the present invention provides a compound of formula (I):

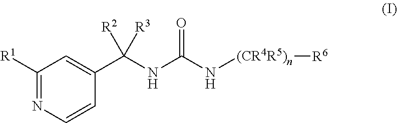

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In some preferred embodiments n is 0.

In some embodiments n is 1.

In some embodiments, wherein n is 1, R⁴ and R⁵ are hydrogen.

In one embodiment, the invention provides a compound of formula (I), or a solvate or a pharmaceutically acceptable salt thereof, wherein R⁶ is a 7-11 membered spirocyclic cycloalkyl, which spirocyclic cycloalkyl is optionally substituted with one, two, or three substituents independently selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In one embodiment, the invention provides a compound of formula (I), or a solvate or a pharmaceutically acceptable salt thereof, wherein R⁶ is a 7-11 membered spirocyclic cycloalkyl, which spirocyclic cycloalkyl is optionally substituted with one substituent selected from halogen, C₁₋₆ alkyl, C₃₋₆cycloalkyl, C₁₋₆ alkoxy, cyano, oxo, hydroxy, hydroxyC₁₋₆alkyl, and haloC₁₋₆alkyl.

In some embodiments, wherein R⁶ is a 7-11 membered spirocyclic cycloalkyl as described herein, the compound of formula (I) is not:

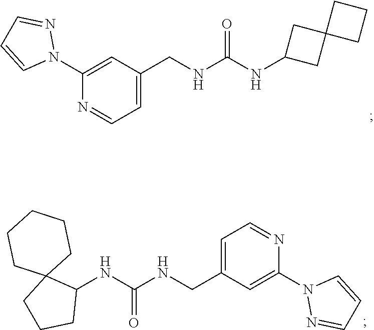 [SMILES: O=C(NCc1ccnc(-n2cccn2)c1)NC1CC2(CCC2)C1 ; O=C(NCc1ccnc(-n2cccn2)c1)NC1CCCC12CCCCC2]

or

In some embodiments, wherein R⁶ is a 7-11 membered spirocyclic cycloalkyl as described herein, the compound of formula (I) excludes:

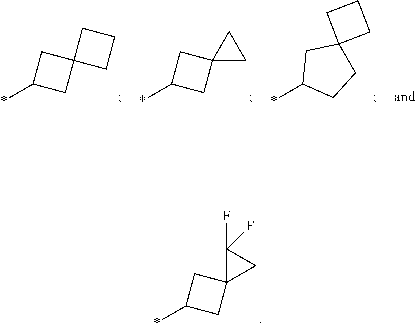 [SMILES: *C1CC2(CCC2)C1 ; *C1CC2(CC2)C1 ; *C1CCC2(CCC2)C1 ; *C1CC2(C1)CC2(F)F]

In embodiments of the invention wherein R⁶ is not selected from;

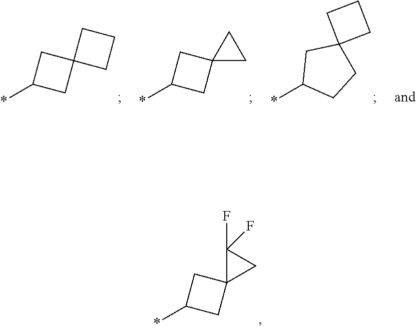 [SMILES: *C1CC2(CCC2)C1 ; *C1CC2(CC2)C1 ; *C1CCC2(CCC2)C1 ; *C1CC2(C1)CC2(F)F]

the following specific compounds may be included as an exception as part of the present compounds:

| | | |
|---|---|---|
| 4 | 1-[[2- (difluoromethoxy)pyridin-4- yl]methyl]-3-spiro[3.3]heptan- 2-ylurea | 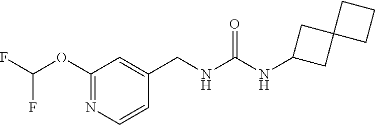 [SMILES: O=C(NCc1ccnc(OC(F)F)c1)NC1CC2(CCC2)C1] |
| 5 | 1-[[2-(2,2- difluoropropoxy)pyridin-4- yl]methyl]-3-spiro[3.3]heptan- 2-ylurea | 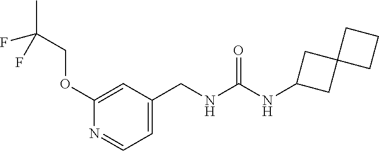 [SMILES: CC(F)(F)COc1cc(CNC(=O)NC2CC3(CCC3)C2)ccn1] |
| 6 | 1-[[2-(2- fluoropropoxy)pyridin-4- yl]methyl]-3-spiro[3.3]heptan- 2-ylurea | 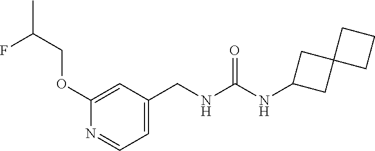 [SMILES: CC(F)COc1cc(CNC(=O)NC2CC3(CCC3)C2)ccn1] |
| 7 | 1-[[2- (cyclopropylmethoxy)pyridin-4- yl]methyl]-3-spiro[3.3]heptan- 2-ylurea | 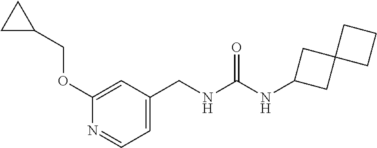 [SMILES: O=C(NCc1ccnc(OCC2CC2)c1)NC1CC2(CCC2)C1] |
| 8 | 1-[[2- (difluoromethoxy)pyridin-4- yl]methyl]-3-spiro[2.3]hexan-5- ylurea | 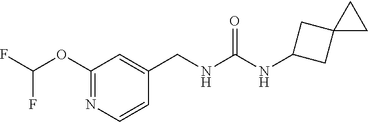 [SMILES: O=C(NCc1ccnc(OC(F)F)c1)NC1CC2(CC2)C1] |
| 9 | 1-[[2-(1,1- difluoroethyl)pyridin-4- yl]methyl]-3-spiro[3.3]heptan- 2-ylurea | 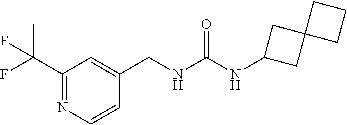 [SMILES: CC(F)(F)c1cc(CNC(=O)NC2CC3(CCC3)C2)ccn1] |
| 10 | 1-spiro[3.3]heptan-2-yl-3-[[2- (trifluoromethyl)pyridin-4- yl]methyl]urea | 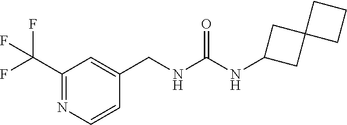 [SMILES: O=C(NCc1ccnc(C(F)(F)F)c1)NC1CC2(CCC2)C1] |
| 11 | 1-[[2-(difluoromethoxy)pyridin- 4-yl]methyl]-3-spiro[3.4]octan- 7-ylurea | 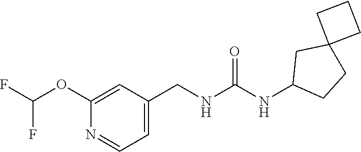 [SMILES: O=C(NCc1ccnc(OC(F)F)c1)NC1CCC2(CCC2)C1] |
| 12 | 1-spiro[2.3]hexan-5-yl-3-[2- (2,2,2-trifluoroethoxy)pyridin- 4-yl]methyl]urea | 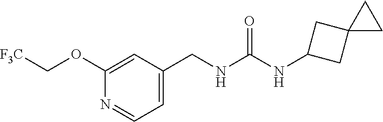 [SMILES: O=C(NCc1ccnc(OCC(F)(F)F)c1)NC1CC2(CC2)C1] |
| 13 | 1-[2-hydroxy-1-[2-(2,2,2- trifluoroethoxy)pyridin-4- yl]ethyl]-3-spiro[3.3]heptan-2- ylurea | 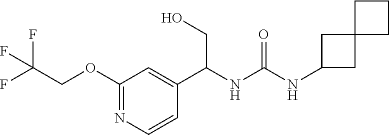 [SMILES: O=C(NC1CC2(CCC2)C1)NC(CO)c1ccnc(OCC(F)(F)F)c1] |
| 15 | 1-[[2- (difluoromethoxy)pyridin-4- yl]methyl]-3-(2,2- difluorospiro[2.3]hexan-5- yl)urea | 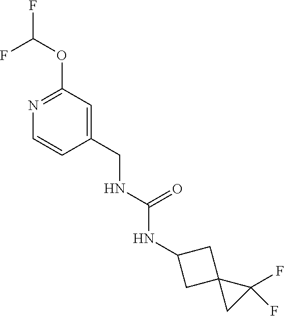 [SMILES: O=C(NCc1ccnc(OC(F)F)c1)NC1CC2(C1)CC2(F)F] |
| 16 | 1-[[2-(difluoromethoxy)pyridin- 4-yl]methyl]-3-(2- fluorospiro[3.3]heptan-6-yl)urea | 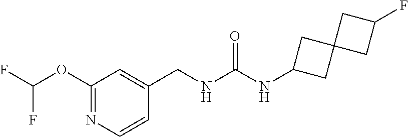 [SMILES: O=C(NCc1ccnc(OC(F)F)c1)NC1CC2(CC(F)C2)C1] |
| 28 | 1-[[2- (difluoromethoxy)pyridin-4- yl]methyl]-3-[(2R,3S)-3- hydroxyspiro[3.3]heptan-2- yl]urea | 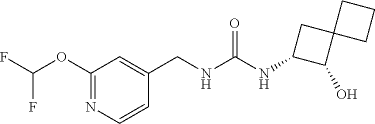 [SMILES: O=C(NCc1ccnc(OC(F)F)c1)N[C@@H]1CC2(CCC2)[C@@H]1O] |
| 29 | 1-[[2-(2,3- difluoropropoxy)pyridin-4- yl]methyl]-3-spiro[3.3]heptan- 2-ylurea | 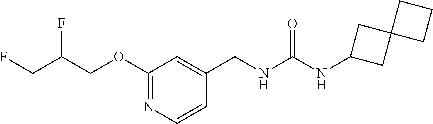 [SMILES: O=C(NCc1ccnc(OCC(F)CF)c1)NC1CC2(CCC2)C1] |
| 31 | 1-[[2- (difluoromethoxy)pyridin-4- yl]methyl]-3-(2,2- difluorospiro[2.5]octan-6- yl)urea | 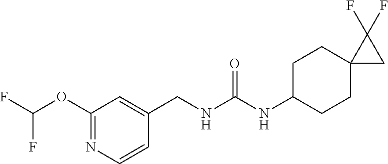 [SMILES: O=C(NCc1ccnc(OC(F)F)c1)NC1CCC2(CC1)CC2(F)F] |

In some embodiments solvates of the compounds of formula (I) are excluded.

In one embodiment, this invention relates to a compound of formula (I):

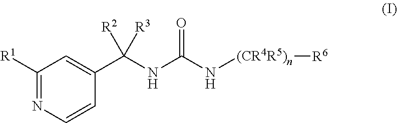

wherein
R¹ is selected from a 5-6 membered heteroaryl, haloC₁₋₆alkoxy, C₃₋₅cycyloxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
or a solvate or a pharmaceutically acceptable salt thereof.

In one embodiment, this invention relates to a compound of formula (I):

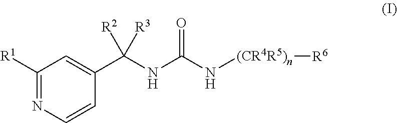

wherein
R¹ is selected from a 5-6 membered heteroaryl, haloC₁₋₆alkoxy, C₃₋₅cycloalkylC₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In one embodiment, this invention relates to a compound of formula (I):

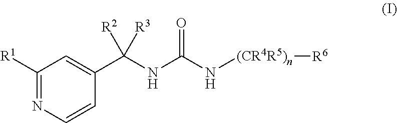

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

In some embodiments, the invention relates to a compound as described herein, wherein R¹ is selected from 5 membered heteroaryl, haloC₁₋₆alkoxy, and haloC₁₋₆alkyl.

In some embodiments, the invention relates to a compound as described herein, wherein R¹ is unsubstituted 5-6 membered heteroaryl.

In some embodiments, the invention relates to a compound as described herein, wherein R¹ is unsubstituted pyrazolyl.

In some preferred embodiments, the invention relates to a compound as described herein, wherein R¹ is haloC₁₋₆alkoxy selected from FCH₂CHFCH₂O—, CHF₂O—, CH₃CF₂O—, CH₃CFHCH₂O— and CF₃CH₂O—. The haloC₁₋₆alkoxy may be linear or branched as described above.

In some preferred embodiments, the invention relates to a compound as described herein, wherein R¹ is haloC₁₋₆alkoxy selected from CHF₂O—, CH₃CF₂O—, CH₃CFHCH₂O—, and CF₃CH₂O—.

In some preferred embodiments, the invention relates to a compound as described herein, wherein R¹ is haloC₁₋₆alkyl selected from CHF₂—, CF₃—, FCH₂CFHCH₂—, and CF₃CH₂—.

In some embodiments, the invention relates to a compound as described herein, wherein (i) both R² and R³ are H, or (ii) R² is HOCH₂— and R³ is H.

In some preferred embodiments, the invention relates to a compound as described herein, wherein n is 0.

In some embodiments, the invention relates to a compound as described herein, wherein n is 1. 1.

In some embodiments, the invention relates to a compound as described herein, wherein one or both of R⁴ and R⁵ are halogen.

In some embodiments, the invention relates to a compound as described herein, wherein both of R⁴ and R⁵ are halogen.

In some embodiments, the invention relates to a compound as described herein, wherein halogen is F—.

In some embodiments, the invention relates to a compound as described herein, wherein one or both of R⁴ and R⁵ are H.

In some embodiments, the invention relates to a compound as described herein, wherein both of R⁴ and R⁵ are H.

In preferred embodiments, n is 0.

If n is 0, the NH group is directly linked to R⁶.

In some embodiments, the invention relates to a compound as described herein, wherein R⁶ is substituted with one or two substituents independently selected from F—, hydroxy, and hydroxyC₁₋₆alkyl.

In some embodiments the invention relates to a compound as described herein, wherein R⁶ is substituted with one or two substituents independently selected from F— and hydroxyC₁₋₆alkyl.

In some embodiments, the invention relates to a compound as described herein, wherein R⁶ is selected from a group consisting of:

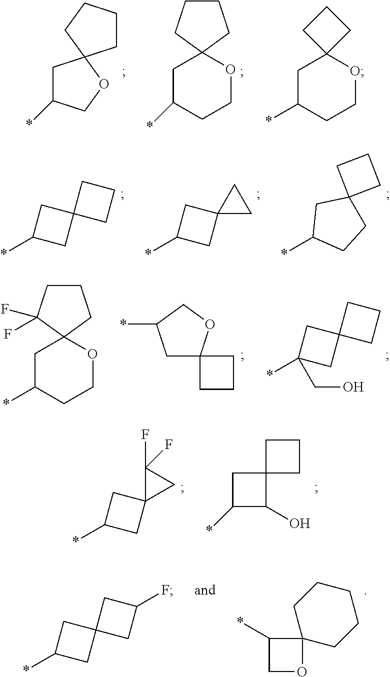

In some embodiments, the invention relates to a compound as described herein, wherein R⁶ is selected from a group consisting of:

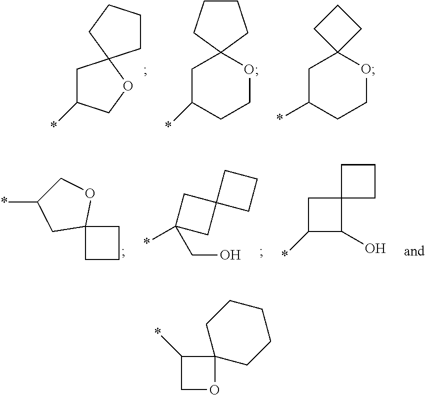

In some embodiments, the invention relates to a compound as described herein, wherein R⁶ is selected from a group consisting of:

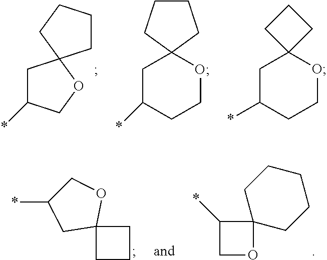

In some embodiments, the invention relates to a compound as described herein, wherein R⁶ is selected from a group consisting of:

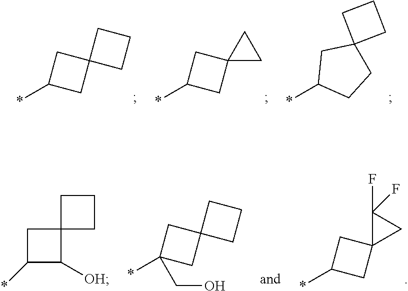

In some embodiments, the invention relates to a compound as described herein, wherein R¹ is CHF₂O—, R² and are R³ are H, n is 0 and R⁶ is selected from the group consisting of:

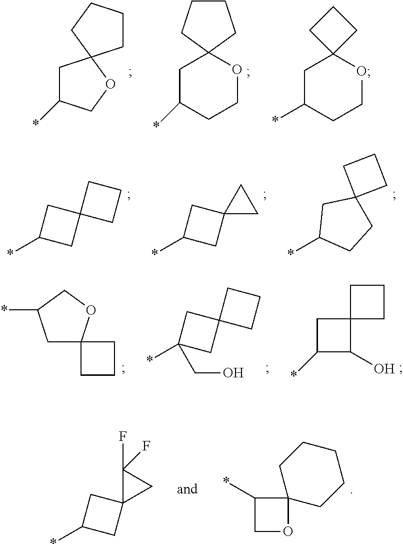

In some preferred embodiments, the invention relates to a compound as described herein, wherein R¹ is CHF₂O—, R² and are R³ are H, n is 0 and R⁶ is selected from the group consisting of:

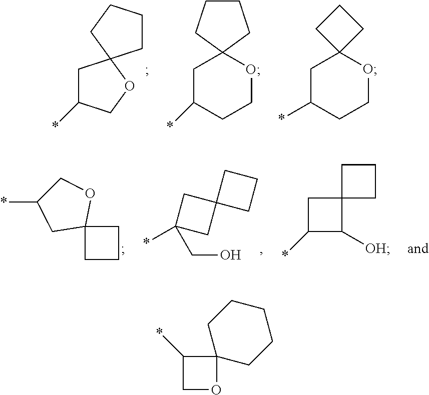

In some more preferred embodiments, the invention relates to a compound as described herein, wherein R¹ is CHF₂O—, R² and are R³ are H, n is 0 and R⁶ is selected from the group consisting of:

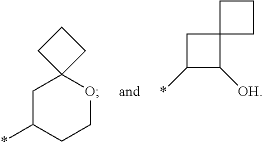 [SMILES: *C1CCOC2(CCC2)C1 ; "and" ; *C1CC2(CCC2)C1O]

In some embodiments, the invention relates to a compound as described herein, wherein R⁶ is not selected from:

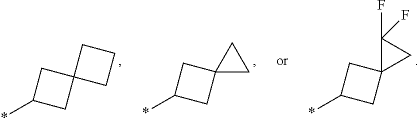 [SMILES: *C1CC2(CCC2)C1 ; *C1CC2(CC2)C1 ; *C1CC2(C1)CC2(F)F]

In some embodiments, the invention relates to a compound as described herein, selected from Table 1.

| Example | Name | Structure | Kv7.2 EC₅₀ (μM) |
|---|---|---|---|
| 1 | 1-(1- oxaspiro[4.4] nonan-3-yl)-3- [(2-pyrazol-1- ylpyridin-4- yl)methyl]urea | 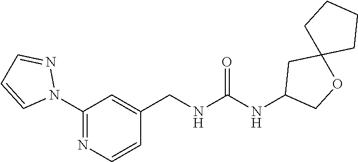 [SMILES: O=C(NCc1ccnc(-n2cccn2)c1)NC1COC2(CCCC2)C1] | 3.63 |
| 2 | 1-(6- oxaspiro[4.5] decan-9-yl)-3-[(2- pyrazol-1- ylpyridin-4- yl)methyl]urea | 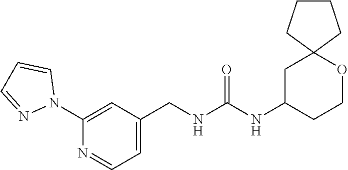 [SMILES: O=C(NCc1ccnc(-n2cccn2)c1)NC1CCOC2(CCCC2)C1] | 8.49 |
| 3 | 1-[[2- (difluoromethoxy) pyridin-4- yl]methyl]-3-(1- oxaspiro[4.4] nonan-3-yl)urea | 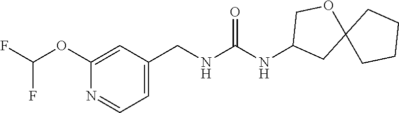 [SMILES: O=C(NCc1ccnc(OC(F)F)c1)NC1COC2(CCCC2)C1] | 0.50 |
| 4 | 1-[[2- (difluoromethoxy) pyridin-4- yl]methyl]-3- spiro[3.3]heptan- 2-ylurea | 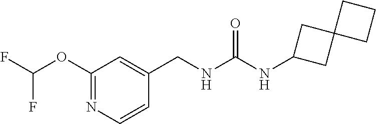 [SMILES: O=C(NCc1ccnc(OC(F)F)c1)NC1CC2(CCC2)C1] | 0.02 |
| 5 | 1-[[2-(2,2- difluoropropoxy) pyridin-4- yl]methyl]-3- spiro[3.3]heptan- 2-ylurea | 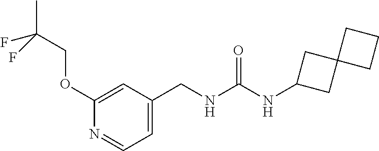 [SMILES: CC(F)(F)COc1cc(CNC(=O)NC2CC3(CCC3)C2)ccn1] | 0.02 |
| 6 | 1-[[2-(2- fluoropropoxy) pyridin-4- yl]methyl]-3- spiro[3.3]heptan- 2-ylurea | 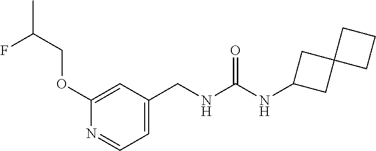 [SMILES: CC(F)COc1cc(CNC(=O)NC2CC3(CCC3)C2)ccn1] | 0.06 |
| 7 | 1-[[2- (cyclopropylmethoxy) pyridin-4- yl]methyl]-3- spiro[3.3]heptan- 2-ylurea | 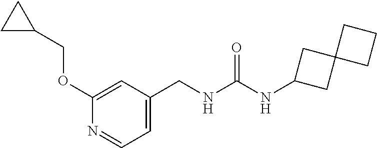 [SMILES: O=C(NCc1ccnc(OCC2CC2)c1)NC1CC2(CCC2)C1] | 0.05 |
| 8 | 1-[[2- (difluoromethoxy) pyridin-4- yl]methyl]-3- spiro[2.3]hexan- 5-ylurea | 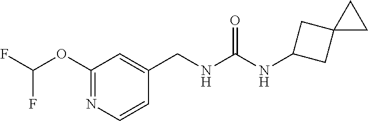 [SMILES: O=C(NCc1ccnc(OC(F)F)c1)NC1CC2(CC2)C1] | 0.06 |
| 9 | 1-[[2-(1,1- difluoroethyl) pyridin-4-yl]methyl]- 3- spiro[3.3]heptan- 2-ylurea | 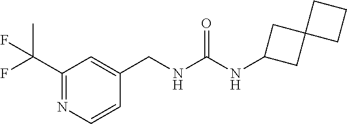 [SMILES: CC(F)(F)c1cc(CNC(=O)NC2CC3(CCC3)C2)ccn1] | 0.19 |
| 10 | 1- spiro[3.3]heptan- 2-yl-3-[[2- (trifluoromethyl) pyridin-4- yl]methyl]urea | 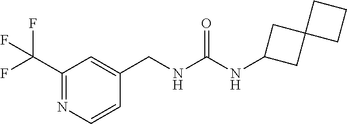 [SMILES: O=C(NCc1ccnc(C(F)(F)F)c1)NC1CC2(CCC2)C1] | 0.19 |
| 11 | 1-[[2- (difluoromethoxy) pyridin-4- yl]methyl]-3- spiro[3.4]octan- 7-ylurea | 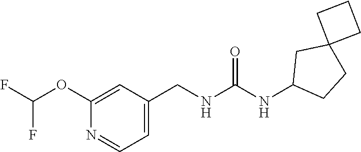 [SMILES: O=C(NCc1ccnc(OC(F)F)c1)NC1CCC2(CCC2)C1] | 0.02 |
| 12 | 1- spiro[2.3]hexan- 5-yl-3-[[2-(2,2,2- trifluoroethoxy) pyridin-4- yl]methyl]urea | 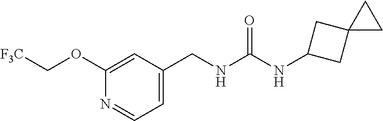 [SMILES: O=C(NCc1ccnc(OCC(F)(F)F)c1)NC1CC2(CC2)C1] | 0.17 |
| 13 | 1-[2-hydroxy-1- [2-(2,2,2- trifluoroethoxy) pyridin-4- yl]ethyl]-3- spiro[3.3]heptan- 2-ylurea | 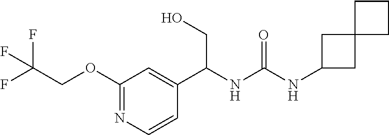 [SMILES: O=C(NC1CC2(CCC2)C1)NC(CO)c1ccnc(OCC(F)(F)F)c1] | 0.07 |
| 14 | 1-[[2- (difluoromethoxy) pyridin-4- yl]methyl]-3-(6- oxaspiro[4.5] decan-9-yl)urea | 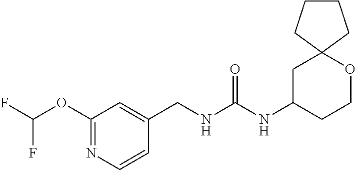 [SMILES: O=C(NCc1ccnc(OC(F)F)c1)NC1CCOC2(CCCC2)C1] | 0.26 |
| 15 | 1-[[2- (difluoromethoxy) pyridin-4- yl]methyl]-3- (2,2- difluorospiro[2.3] hexan-5-yl)urea | 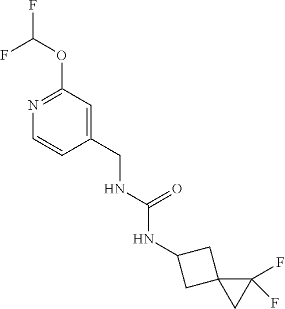 [SMILES: O=C(NCc1ccnc(OC(F)F)c1)NC1CC2(C1)CC2(F)F] | 3.10 |
| 16 | 1-[[2- (difluoromethoxy) pyridin-4- yl]methyl]-3-(2- fluorospiro[3.3] heptan-6-yl)urea | 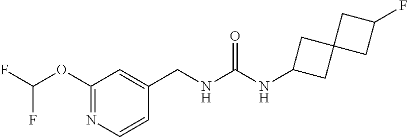 [SMILES: O=C(NCc1ccnc(OC(F)F)c1)NC1CC2(CC(F)C2)C1] | 0.15 |
| 17 | 1-[[2- (difluoromethoxy) pyridin-4- yl]methyl]-3-(5- oxaspiro[3.5] nonan-8-yl)urea | 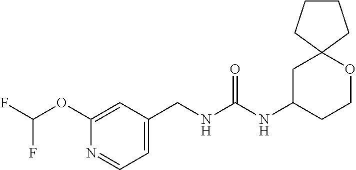 [SMILES: O=C(NCc1ccnc(OC(F)F)c1)NC1CCOC2(CCCC2)C1] | 0.34 |
| 18 | 1-[[2- (difluoromethoxy) pyridin-4- yl]methyl]-3-[2- (hydroxymethyl) spiro[3.3]heptan- 2-yl]urea | 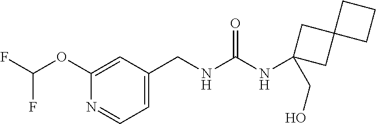 [SMILES: O=C(NCc1ccnc(OC(F)F)c1)NC1(CO)CC2(CCC2)C1] | 0.41 |
| 19 | 1-[[2- (difluoromethoxy) pyridin-4- yl]methyl]-3- (2,2- difluorospiro[2.4] heptan-6-yl)urea | 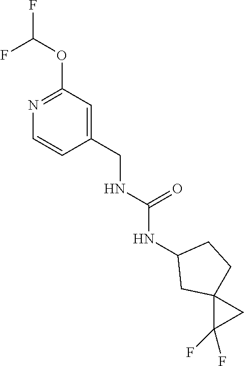 [SMILES: O=C(NCc1ccnc(OC(F)F)c1)NC1CCC2(C1)CC2(F)F] | 0.35 |
| 20 | 1-[[2- (difluoromethoxy) pyridin-4- yl]methyl]-3-(1- oxaspiro[3.5] nonan-3-yl)urea | 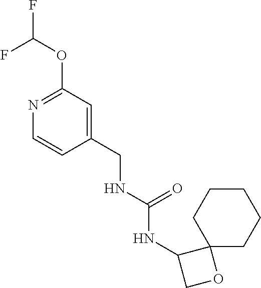 [SMILES: O=C(NCc1ccnc(OC(F)F)c1)NC1COC12CCCCC2] | 8.43 |
| 21 | 1-[[2- (difluoromethoxy) pyridin-4- yl]methyl]-3- (4,4-difluoro-6- oxaspiro[4.5] decan-9-yl)urea | 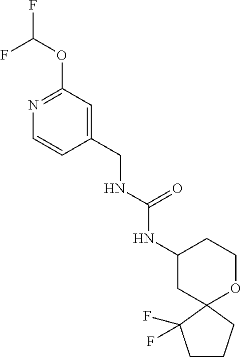 [SMILES: O=C(NCc1ccnc(OC(F)F)c1)NC1CCOC2(CCCC2(F)F)C1] | 0.21 |
| 22 | 1-[[2- (difluoromethoxy) pyridin-4- yl]methyl]-3-(5- oxaspiro[3.4] octan-7-yl)urea | 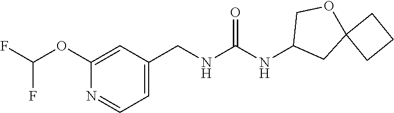 [SMILES: O=C(NCc1ccnc(OC(F)F)c1)NC1COC2(CCC2)C1] | 1.44 |
| 23 | 1-[[2- (difluoromethoxy) pyridin-4- yl]methyl]-3- spiro[2.3]hexan- 2-ylurea | 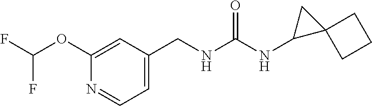 [SMILES: O=C(NCc1ccnc(OC(F)F)c1)NC1CC12CCC2] | 1.94 |
| 24 | 1-[[2- (difluoromethoxy) pyridin-4- yl]methyl]-3- (2,2- difluorospiro[2.3] hexan-1-yl)urea | 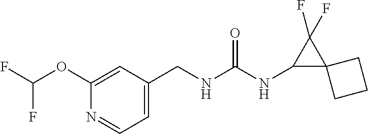 [SMILES: O=C(NCc1ccnc(OC(F)F)c1)NC1C(F)(F)C12CCC2] | 0.34 |
| 25 | 1-[[2- (difluoromethoxy) pyridin-4- yl]methyl]-3- [rac-(2R,3R)-3- hydroxyspiro[3.3] heptan-2-yl]urea | 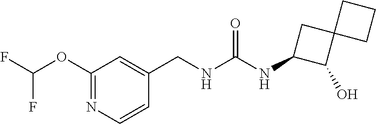 [SMILES: O=C(NCc1ccnc(OC(F)F)c1)N[C@H]1CC2(CCC2)[C@@H]1O] | 0.74 |
| 26 | 1-[[2- (difluoromethoxy) pyridin-4- yl]methyl]-3- [rac-(2R,3S)-3- hydroxyspiro[3.3] heptan-2-yl]urea | 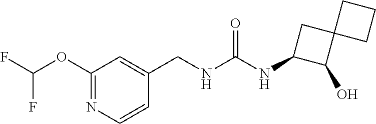 [SMILES: O=C(NCc1ccnc(OC(F)F)c1)N[C@H]1CC2(CCC2)[C@H]1O] | 0.44 |
| 27 | 1-[[2- (difluoromethoxy) pyridin-4- yl]methyl]-3- [(8R)-5- oxaspiro[3.5] nonan-8-yl]urea | 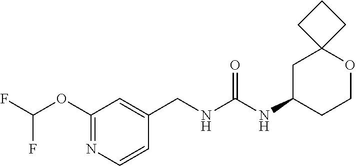 [SMILES: O=C(NCc1ccnc(OC(F)F)c1)N[C@@H]1CCOC2(CCC2)C1] | 0.26 |
| 28 | 1-[[2- (difluoromethoxy) pyridin-4- yl]methyl]-3- [(2R,3S)-3- hydroxyspiro[3.3] heptan-2-yl]urea | 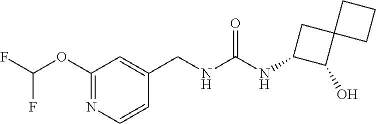 [SMILES: O=C(NCc1ccnc(OC(F)F)c1)N[C@@H]1CC2(CCC2)[C@@H]1O] | 0.50 |
| 29 | 1-[[2-(2,3- difluoropropoxy) pyridin-4- yl]methyl]-3- spiro[3.3]heptan- 2-ylurea | 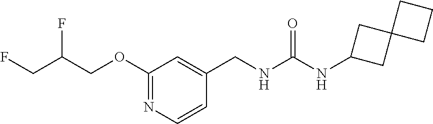 [SMILES: O=C(NCc1ccnc(OCC(F)CF)c1)NC1CC2(CCC2)C1] | 0.10 |
| 30 | 1-[[2- (difluoromethoxy) pyridin-4- yl]methyl]-3-(1- oxaspiro[4.5] decan-3-yl)urea | 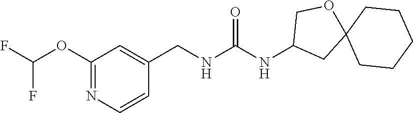 [SMILES: O=C(NCc1ccnc(OC(F)F)c1)NC1COC2(CCCCC2)C1] | 0.45 |
| 31 | 1-[[2- (difluoromethoxy) pyridin-4- yl]methyl]-3- (2,2- difluorospiro[2.5] octan-6-yl)urea | 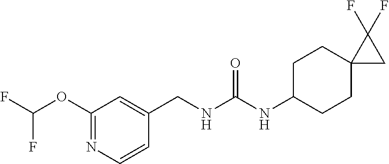 [SMILES: O=C(NCc1ccnc(OC(F)F)c1)NC1CCC2(CC1)CC2(F)F] | 0.14 |
| 32 | 1-[[2- (difluoromethoxy) pyridin-4- yl]methyl]-3-(5- oxaspiro[3.5] nonan-7-yl)urea | 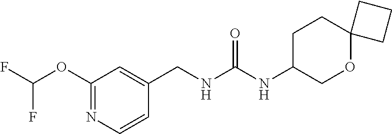 [SMILES: O=C(NCc1ccnc(OC(F)F)c1)NC1CCC2(CCC2)OC1] | 0.55 |

In some embodiments, the invention relates to a compound as described herein, wherein such compounds of formula (I) show Kv7.2 EC₅₀: <1 μM or <3 μM as most preferred or preferred EC₅₀. In some embodiments such compounds show Kv7.2 EC₅₀: <3 μM.

In some embodiments, the invention relates to a compound as described herein, wherein such compounds of formula (I) show a favorable selectivity (≥10-fold) for Kv7.2 over Kv7.5/7.3 as shown in Table A below.

TABLE A

| | | | Kv7.5/ | Kv7.5/7.3/ |
|---|---|---|---|---|
| | | Kv7.2 | 7.3 | Kv7.2 |
| Exam- | | EC₅₀ | EC₅₀ | selectivity |
| ple | Name | (μM) | (μM) | ratio |
| 14 | 1-[[2-(difluoromethoxy)pyridin- | 0.26 | >30 | >116 |
| | 4-yl]methyl]-3-(6- | | | |
| | oxaspiro[4.5]decan-9-yl)urea | | | |
| 17 | 1-[[2- | 0.34 | >30 | >87 |
| | (difluoromethoxy)pyridin-4- | | | |
| | yl]methyl]-3-(5- | | | |
| | oxaspiro[3.5]nonan-8-yl)urea | | | |
| 19 | 1-[[2- | 0.35 | >30 | >86 |
| | (difluoromethoxy)pyridin-4- | | | |
| | yl]methyl]-3-(2,2- | | | |
| | difluorospiro[2.4]heptan-6- | | | |
| | yl)urea | | | |
| 21 | 1-[[2- | 0.21 | >30 | >146 |
| | (difluoromethoxy)pyridin-4- | | | |
| | yl]methyl]-3-(4,4-difluoro-6- | | | |
| | oxaspiro[4.5]decan-9-yl)urea | | | |
| 23 | 1-[[2-(difluoromethoxy)pyridin- | 1.94 | >30 | >15 |
| | 4-yl]methyl]-3-spiro[2.3 ]hexan- | | | |
| | 2-ylurea | | | |
| 24 | 1-[[2-(difluoromethoxy)pyridin- | 0.34 | >30 | >89 |
| | 4-yl]methyl]-3-(2,2- | | | |
| | difluorospiro[2.3]hexan-1- | | | |
| | yl)urea | | | |
| 25 | 1-[[2-(difluoromethoxy)pyridin- | 0.74 | >30 | >41 |
| | 4-yl]methyl]-3-[rac-(2R,3R)-3- | | | |
| | hydroxyspiro[3.3]heptan-2- | | | |
| | yl]urea | | | |
| 27 | 1-[[2- | 0.26 | >30 | >115 |
| | (difluoromethoxy)pyridin-4- | | | |
| | yl]methyl]-3-[(8R)-5- | | | |
| | oxaspiro[3.5]nonan-8-yl]urea | | | |
| 28 | 1-[[2- | 0.50 | >30 | >60 |
| | (difluoromethoxy)pyridin-4- | | | |
| | yl]methyl]-3-[(2R,3S)-3- | | | |
| | hydroxyspiro[3.3]heptan-2- | | | |
| | yl]urea | | | |

In some embodiments, the invention relates to a compound as described herein, wherein such compounds of formula (I) show favorable human liver microsomal clearance (<20 l/min/mg) as shown in Table B below.

TABLE B

| | | Human liver |
|---|---|---|
| | | microsomal intrinsic |
| Exam- | | clearance CLint |
| ple | Name | (μl/min/mg) |
| 8 | 1-[[2-(difluoromethoxy)pyridin-4- | 5.2 |
| | yl]methyl]-3-spiro[2.3]hexan-5-ylurea | |
| 9 | 1-[[2-(1,1-difluoroethyl)pyridin-4- | 17.6 |
| | yl]methyl]-3-spiro[3.3]heptan-2-ylurea | |
| 12 | 1-spiro[2.3]hexan-5-yl-3-[2-(2,2,2- | 15.2 |
| | trifluoroethoxy)pyridin-4-yl]methyl]urea | |
| 14 | 1-[[2-(difluoromethoxy)pyridin-4- | 20.0 |
| | yl]methyl]-3-(6-oxaspiro[4.5]decan-9- | |
| | yl)urea | |
| 15 | 1-[[2-(difluoromethoxy)pyridin-4- | 11.3 |
| | yl]methyl]-3-(2,2-difluorospiro[2.3]hexan- | |
| | 5-yl)urea | |
| 16 | 1-[[2-(difluoromethoxy)pyridin-4- | 10.7 |
| | yl]methyl]-3-(2-fluorospiro[3.3]heptan-6- | |
| | yl)urea | |
| 17 | 1-[[2-(difluoromethoxy)pyridin-4- | 3.8 |
| | yl]methyl]-3-(5-oxaspiro[3.5]nonan-8- | |
| | yl)urea | |
| 21 | 1-[2-(difluoromethoxy)pyridin-4- | 20.4 |
| | yl]methyl]-3-(4,4-difluoro-6- | |
| | oxaspiro[4.5]decan-9-yl)urea | |
| 22 | 1-[[2-(difluoromethoxy)pyridin-4- | 10.6 |
| | yl]methyl]-3-(5-oxaspiro[3.4]octan-7- | |
| | yl)urea | |
| 25 | 1-[[2-(difluoromethoxy)pyridin-4- | 14.2 |
| | yl]methyl]-3-[rac-(2R,3R)-3- | |
| | hydroxyspiro[3.3]heptan-2-yl]urea | |
| 26 | 1-[[2-(difluoromethoxy)pyridin-4- | 11.5 |
| | yl]methyl]-3-[rac-(2R,3S)-3- | |
| | hydroxyspiro[3.3]heptan-2-yl]urea | |
| 27 | 1-[[2-(difluoromethoxy)pyridin-4- | 20.5 |
| | yl]methyl]-3-[(8R)-5-oxaspiro[3.5]nonan- | |
| | 8-yl]urea | |
| 31 | 1-[[2-(difluoromethoxy)pyridin-4- | 18.8 |
| | yl]methyl]-3-(2,2-difluorospiro[2.5]octan- | |
| | 6-yl)urea | |

In some embodiments, the invention relates to a compound as described herein, wherein such compounds of formula (I) show minimal activity on Kv7.4 (EC₅₀>30 μM) and a favorable selectivity (>10-fold) for Kv7.2 over Kv7.4 as shown in Table C below.

TABLE C

| | | Kv7.2 | Kv7.4 | Kv7.4/Kv7.2 |
|---|---|---|---|---|
| Exam- | | EC₅₀ | EC₅₀ | selectivity |
| ple | Name | (μM) | (μM) | ratio |
| 21 | 1-[[2- | 0.21 | >30 | >143 |
| | (difluoromethoxy)pyridin-4- | | | |
| | yl]methyl]-3-(4,4-difluoro-6- | | | |
| | oxaspiro[4.5]decan-9-yl)urea | | | |
| 27 | 1-[[2- | 0.26 | >30 | >115 |
| | (difluoromethoxy)pyridin-4- | | | |
| | yl]methyl]-3-[(8R)-5- | | | |
| | oxaspiro[3.5]nonan-8-yl]urea | | | |

In some embodiments, the invention relates to a compound, or a pharmaceutical composition as described herein, for use in the therapeutic and/or prophylactic treatment of a disorder, disease, or disability associated with Kv7.2 in a subject in need thereof.

In some embodiments, the invention relates to a compound, or a pharmaceutical composition as described herein; for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In some embodiments, the invention relates to a compound, or a pharmaceutical composition for use as described herein, wherein the disorder, disease, or disability is a behavioral disorder which is Attention Deficit Hyperactivity Disorder (ADHD).

In some embodiments, the invention relates to a compound or pharmaceutical composition for use as described herein, wherein the disorder, disease, or disability, wherein the disorder, disease, or disability is a neurodevelopment disorder selected from autism spectrum disorder (ASD) and syndromic developmental disorders.

In some embodiment, the invention relates to a compound or pharmaceutical composition for use as described herein, wherein the disorder, disease, or disability is a syndromic developmental disorder is selected from Dup15q syndrome (Dup15q), Fragile X syndrome (FXS) and Angelman syndrome.

In some embodiments, the invention relates to a compound or pharmaceutical composition for use as described herein, wherein the disorder, disease, or disability is an epilepsy selected from broad pediatric epilepsy, West syndrome, Ohtahara syndrome, and epileptic encephalopathy.

In some embodiments, the invention relates to a compound or pharmaceutical composition for use as described herein, wherein the disorder, disease, or disability is a neurodegenerative disease selected from Alzheimer's disease, and motor neuron diseases.

In some embodiments, the invention relates to a compound or pharmaceutical compositions for use as described herein, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

In some embodiments, the invention relates to a compound or a pharmaceutical composition as described herein, for use in therapy.

In some embodiments, the invention relates to a compound, or a pharmaceutical composition as described herein, for the manufacture of a medicament for use in the therapeutic and/or prophylactic treatment of a disorder, disease, or disability associated with Kv7.2.

In some embodiments, the invention relates to a method for the therapeutic and/or prophylactic treatment of a disorder, disease, or disability associated with Kv7.2, which method comprises administering a therapeutically effective amount of a compound, or a pharmaceutical composition as described herein, for use as described herein.

In some embodiments, the invention relates to a kit for use in the therapeutic and/or prophylactic treatment of a disorder, disease, or disability associated with Kv7.2 comprising:
a) a compound of this invention; and
b) instructions for use.

In some embodiments, the invention is as hereinbefore described.

In one embodiment, the invention provides a compound of formula (I), or a solvate or a pharmaceutically acceptable salt thereof, wherein R¹ is a 5-6 membered heteroaryl, a C₃₋₅cycloalkyl-C₁₋₆alkoxy, a haloC₁₋₆alkyl or a haloC₁₋₆alkoxy.

In one preferred embodiment, the invention provides a compound of formula (I), or a solvate or a pharmaceutically acceptable salt thereof, wherein R¹ is a haloC₁₋₆alkyl or a haloC₁₋₆alkoxy.

In one embodiment, the invention provides a compound of formula (I), or a solvate or a pharmaceutically acceptable salt thereof, wherein R¹ is a 5-6 membered heteroaryl,

In some embodiments, the invention relates to any medical use of a compound as described herein, wherein R¹ is C₃₋₅cycloalkyl-C₁₋₆alkoxy.

In one embodiment, the invention provides a compound of formula (I), or a solvate or a pharmaceutically acceptable salt thereof, wherein R¹ is a 5-6 membered heteroaryl, a haloC₁₋₆alkyl or a haloC₁₋₆alkoxy, R² and R³ are H, and n is 0.

In one preferred embodiment, the invention provides a compound of formula (I), or a solvate or a pharmaceutically acceptable salt thereof, wherein R¹ is a haloC₁₋₆alkyl or a haloC₁₋₆alkoxy and R² and R³ are H.

In one embodiment, the invention provides a compound of formula (I), or a solvate or a pharmaceutically acceptable salt thereof, wherein R¹ is a haloC₁₋₆alkyl or a haloC₁₋₆alkoxy and R² is H and R³ is hydroxyC₁₋₆alkyl.

In one embodiment, the invention provides a compound of formula (I), or a solvate or a pharmaceutically acceptable salt thereof, wherein R¹ is a 5-6 membered heteroaryl, R² and R³ are H, or R² is H and R³ is hydroxyC₁₋₆alkyl, and n is 0.

In one embodiment, the invention provides a compound of formula (I), or a solvate or a pharmaceutically acceptable salt thereof, wherein R¹ is a 5-6 membered heteroaryl, R² and R³ are H, and n is 0.

In one preferred embodiment, the invention provides a compound of formula (I), or a solvate or a pharmaceutically acceptable salt thereof, wherein R¹ is a haloC₁₋₆alkyl or a haloC₁₋₆alkoxy, R² and R³ are H, and n is 0.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one, two, or three substituents independently selected from halogen, C₁₋₆alkyl, hydroxy, and hydroxyC₁₋₆alkyl.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one, two, or three substituents independently selected from halogen, hydroxy, and hydroxyC₁₋₆alkyl.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one or two substituents independently selected from halogen, hydroxy, and hydroxyC₁₋₆alkyl.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one, two, or three substituents independently selected from F—, hydroxy, and HOCH₂—.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one or two substituents independently selected from F—, hydroxy, and HOCH₂—.

In one preferred embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one or two hydroxy.

In one preferred embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is optionally substituted with one hydroxy.

In one embodiment, the invention provides a compound of formula (I), or a solvate or a pharmaceutically acceptable salt thereof, wherein R¹, R², R³, n, R⁴, and R⁵ are as described herein, and R⁶ is a 7-11 membered spirocyclic cycloalkyl, which spirocyclic cycloalkyl is unsubstituted.

In one embodiment, the invention provides a compound of formula (I), or a solvate or a pharmaceutically acceptable salt thereof, wherein R⁶ is a 7-11 membered spirocyclic cycloalkyl, selected from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are optionally substituted with one, two, or three substituents independently selected from halogen, C₁₋₆alkyl, hydroxy, and hydroxyC₁₋₆alkyl.

In one embodiment, the invention provides a compound of formula (I), or a solvate or a pharmaceutically acceptable salt thereof, wherein R⁶ is a 7-11 membered spirocyclic cycloalkyl, selected from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are optionally substituted with one, two, or three substituents independently selected from halogen, hydroxy, and hydroxyC₁₋₆alkyl.

In one embodiment, the invention provides a compound of formula (I), or a solvate or a pharmaceutically acceptable salt thereof, wherein R⁶ is a 7-11 membered spirocyclic cycloalkyl, selected from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are optionally substituted with one, two, or three substituents independently selected from F—, CH₃—, hydroxy, and HOCH₂—.

In one embodiment, the invention provides a compound of formula (I), or a solvate or a pharmaceutically acceptable salt thereof, wherein R⁶ is a 7-11 membered spirocyclic cycloalkyl, selected from from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are optionally substituted with one, two, or three substituents independently selected from F—, hydroxy, and HOCH₂—.

In one embodiment, the invention provides a compound of formula (I), or a solvate or a pharmaceutically acceptable salt thereof, wherein R⁶ is a 7-11 membered spirocyclic cycloalkyl, selected from from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are optionally substituted with one or two substituents independently selected from F—, hydroxy, and HOCH₂—.

In one embodiment, the invention provides a compound of formula (I), or a solvate or a pharmaceutically acceptable salt thereof, wherein R⁶ is a 7-11 membered spirocyclic cycloalkyl, selected from from spiro[3.3]heptanyl, spiro[2.3]hexanyl, spiro[2.4]heptanyl, and spiro[3.4]octanyl, spiro[3.3]heptanyl, spiro[2.3]hexanyl, and spiro[3.4]octanyl which are unsubstituted.

In all of the herein embodiments of R⁶ is a 7-11 membered spirocyclic cycloalkyl, R¹, R², R³, R⁴, R⁵ and n are as described herein, i.e. including any combination of R¹, R², R³, R⁴, R⁵ and n are as described herein.

In some of the herein embodiments, R² is H and R³ is hydroxyC₁₋₆alkyl, in particular HOCH₂—.

In some preferred embodiments, n is 0.

In some particularly preferred embodiments, R¹ is haloC₁₋₆alkoxy, R² and R₃ are hydrogen, n is 0, and R⁶ is a 7-11 membered spirocyclic cycloalkyl optionally substituted as described herein.

In some particularly preferred embodiments, R¹ is haloC₁₋₆alkoxy, R² and R₃ are hydrogen, n is 0, and R⁶ is a 7-11 membered spirocyclic cycloalkyl which is substituted as described herein.

In some particularly preferred embodiments, R¹ is haloC₁₋₆alkoxy, R² and R₃ are hydrogen, n is 0, and R⁶ is a 7-11 membered spirocyclic cycloalkyl which is substituted with one substituent as described herein.

In some particularly preferred embodiments, R¹ is haloC₁₋₆alkoxy, R² and R₃ are hydrogen, n is 0, and R⁶ is a 7-11 membered spirocyclic cycloalkyl which is substituted with one hydroxy or F.

In some embodiments, the first ring of the bicyclic spiro cycloalkyl is not a 4 membered ring.

In the following embodiments, R¹, R², R³, R⁴, R⁵ and n are as described herein.

In some embodiments, R⁶ the 7-11 membered spirocycloalkyl has the following structure:

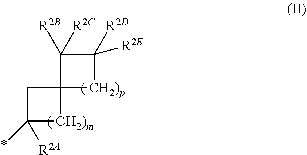

wherein
m is 0, 1, or 2;
p is 0 or 1;
R^{2A}, R^{2B}, R^{2C}; R^{2D}, and R^{2E} are independently selected from H, halogen, C₁₋₆alkyl, hydroxy, and hydroxyC₁₋₆alkyl.

In one embodiment, R^{2A}, R^{2B}, R^{2C}; R^{2D}, and R^{2E} are independently selected from H, halogen, C₁₋₆alkyl, and hydroxyC₁₋₆alkyl.

In one embodiment, R^{2A} is not hydroxy.

In some embodiments, R⁶ being the 7-11 membered spirocycloalkyl has the following structure:

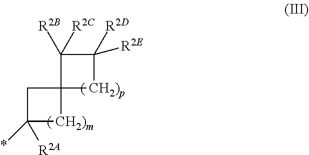

wherein
m is 0, 1, or 2;
p is 0 or 1;
R^{2A} is H or hydroxyC₁₋₆alkyl;
R^{2B}, R^{2C}; R^{2D}, and R^{2E} are independently selected from H, and halogen.

In some embodiments, R⁶ being the 7-11 membered spirocycloalkyl has the following structure:

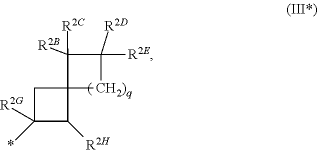

wherein
q is 0 or 1;
R^{2B}, R^{2C}, R^{2D}; R^{2E}, R^{2G} and R^{2H} are independently selected from H, halogen, C₁₋₆alkyl, hydroxy, and hydroxyC₁₋₆alkyl, provided that one of R^{2G} and R^{2H} is not hydrogen.

In one particularly preferred embodiment hydroxyC₁₋₆alkyl is HOCH₂—.

In one particularly preferred embodiment, halogen is F.

In some preferred embodiments, R⁶ being the 7-11 membered spirocycloalkyl has the following structure:

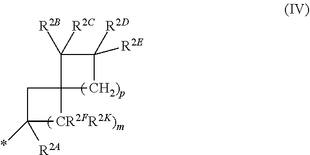

wherein
m is 0, 1, or 2;
p is 0 or 1;
R^{2A} is H or hydroxyC₁₋₆alkyl;
R^{2B}, R^{2C}; R^{2D}, and R^{2E} are independently selected from H, and halogen.
R^{2F} and R^{2K} are independently selected from H, halogen, and hydroxy.

In some preferred embodiments, R⁶ being the 7-11 membered spirocycloalkyl has the following structure:

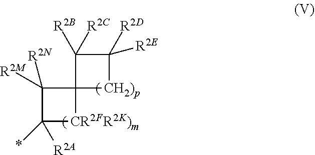

wherein
R^{2A}, R^{2B}, R^{2C}; R^{2D}, R^{2E}, R^{2F}, and R^{2K} are as described above and R^{2M} and R^{2N} are independently selected from H and halogen,
m is 0, 1, or 2; and
p is 0 or 1.

In embodiments wherein m is 0, the CH₂ group is absent and the ring is a 3 membered ring as in Example 24.

In some embodiments, halogen is F.

In some embodiments, R^{2M} and R^{2N} are F.

In some embodiments, R⁶ being the 7-11 membered spirocycloalkyl has the following structure selected from formulae (II) to (V) and R^{2A}, R^{2B}, R^{2C}, R^{2D}, R^{2E}, R^{2F}, R^{2K}, R^{2M} and R^{2N} are as described herein.

In one particularly preferred embodiment hydroxyC₁₋₆alkyl is HOCH₂—.

In one particularly preferred embodiment, halogen is F.

In one particularly preferred embodiment, the m is 1, R^{2F} is hydroxy and R^{2K} is H.

In one embodiment, the invention provides a compound of formula (I), or a solvate or a pharmaceutically acceptable salt thereof, wherein R¹, R², R³, R⁴, R⁵ and n are as described herein, and R⁶ is a 7-11 membered spirocyclic cycloalkyl and non-limiting examples are selected from Table 2.

TABLE 2

| | | |
|---|---|---|
| 4 | 1-[[2- (difluoromethoxy)pyridin-4- yl]methyl]-3-spiro[3.3]heptan- 2-ylurea | 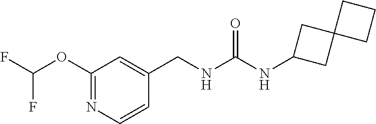 [SMILES: O=C(NCc1ccnc(OC(F)F)c1)NC1CC2(CCC2)C1] |
| 5 | 1-[[2-(2,2- difluoropropoxy)pyridin-4- yl]methyl]-3-spiro[3.3]heptan- 2-ylurea | 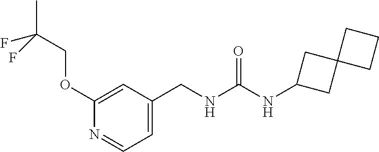 [SMILES: CC(F)(F)COc1cc(CNC(=O)NC2CC3(CCC3)C2)ccn1] |
| 6 | 1-[[2-(2- fluoropropoxy)pyridin-4- yl]methyl]-3-spiro[3.3]heptan- 2-ylurea | 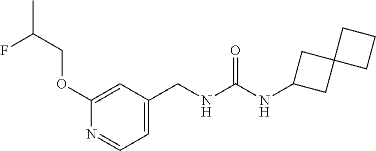 [SMILES: CC(F)COc1cc(CNC(=O)NC2CC3(CCC3)C2)ccn1] |
| 7 | 1-[[2- (cyclopropylmethoxy)pyridin-4- yl]methyl]-3-spiro[3.3]heptan- 2-ylurea | 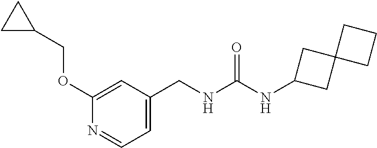 [SMILES: O=C(NCc1ccnc(OCC2CC2)c1)NC1CC2(CCC2)C1] |
| 8 | 1-[[2- (difluoromethoxy)pyridin-4- yl]methyl]-3-spiro[2.3]hexan-5- ylurea | 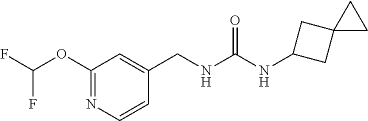 [SMILES: O=C(NCc1ccnc(OC(F)F)c1)NC1CC2(CC2)C1] |
| 9 | 1-[[2-(1,1- difluoroethyl)pyridin-4- yl]methyl]-3-spiro[3.3]heptan- 2-ylurea | 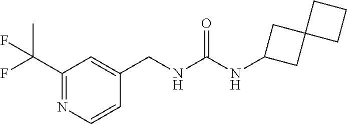 [SMILES: CC(F)(F)c1cc(CNC(=O)NC2CC3(CCC3)C2)ccn1] |
| 10 | 1-spiro[3.3]heptan-2-yl-3-[2- (trifluoromethyl)pyridin-4- yl]methyl]urea | 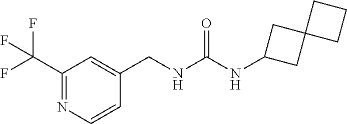 [SMILES: O=C(NCc1ccnc(C(F)(F)F)c1)NC1CC2(CCC2)C1] |
| 11 | 1-[[2-(difluoromethoxy)pyridin- 4-yl]methyl]-3-spiro[3.4]octan- 7-ylurea | 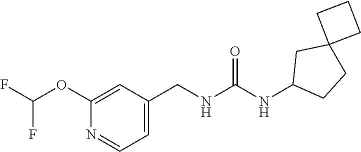 [SMILES: O=C(NCc1ccnc(OC(F)F)c1)NC1CCC2(CCC2)C1] |
| 12 | 1-spiro[2.3]hexan-5-yl-3-[[2- (2,2,2-trifluoroethoxy)pyridin- 4-yl]methyl]urea | 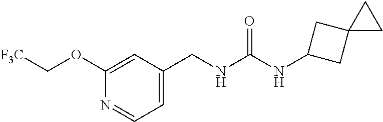 [SMILES: O=C(NCc1ccnc(OCC(F)(F)F)c1)NC1CC2(CC2)C1] |
| 13 | 1-[2-hydroxy-1-[2-(2,2,2- trifluoroethoxy)pyridin-4- yl]ethyl]-3-spiro[3.3]heptan-2- ylurea | 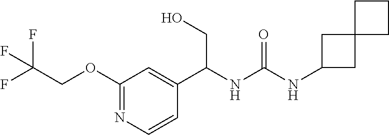 [SMILES: O=C(NC1CC2(CCC2)C1)NC(CO)c1ccnc(OCC(F)(F)F)c1] |
| 15 | 1-[[2- (difluoromethoxy)pyridin-4- yl]methyl]-3-(2,2- difluorospiro[2.3]hexan-5- yl)urea | 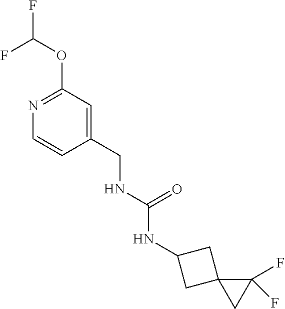 [SMILES: O=C(NCc1ccnc(OC(F)F)c1)NC1CC2(C1)CC2(F)F] |
| 16 | 1-[[2-(difluoromethoxy)pyridin- 4-yl]methyl]-3-(2- fluorospiro[3.3]heptan-6-yl)urea | 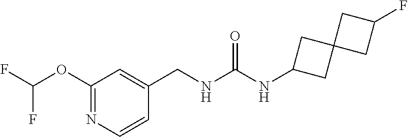 [SMILES: O=C(NCc1ccnc(OC(F)F)c1)NC1CC2(CC(F)C2)C1] |
| 18 | 1-[[2- (difluoromethoxy)pyridin-4- yl]methyl]-3-[2- (hydroxymethyl)spiro[3.3] heptan-2-yl]urea | 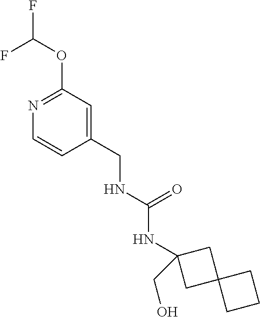 [SMILES: O=C(NCc1ccnc(OC(F)F)c1)NC1(CO)CC2(CCC2)C1] |
| 19 | 1-[[2- (difluoromethoxy)pyridin-4- yl]methyl]-3-(2,2- difluorospiro[2.4]heptan-6- yl)urea | 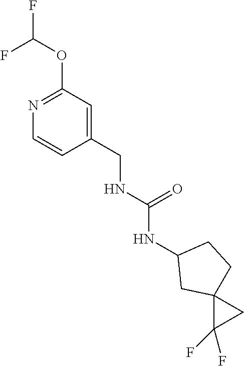 [SMILES: O=C(NCc1ccnc(OC(F)F)c1)NC1CCC2(C1)CC2(F)F] |
| 23 | 1-[[2-(difluoromethoxy)pyridin- 4-yl]methyl]-3-spiro[2.3]hexan- 2-ylurea | 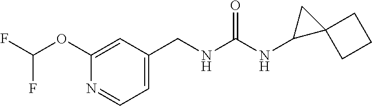 [SMILES: O=C(NCc1ccnc(OC(F)F)c1)NC1CC12CCC2] |
| 24 | 1-[[2-(difluoromethoxy)pyridin- 4-yl]methyl]-3-(2,2- difluorospiro[2.3]hexan-1- yl)urea | 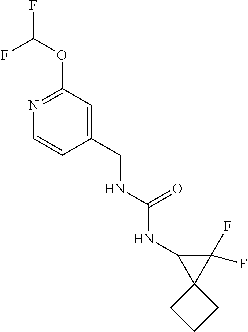 [SMILES: O=C(NCc1ccnc(OC(F)F)c1)NC1C(F)(F)C12CCC2] |
| 25 | 1-[[2-(difluoromethoxy)pyridin- 4-yl]methyl]-3-[rac-(2R,3R)-3- hydroxyspiro[3.3]heptan-2- yl]urea | 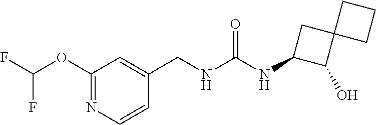 [SMILES: O=C(NCc1ccnc(OC(F)F)c1)N[C@H]1CC2(CCC2)[C@@H]1O] |
| 29 | 1-[[2-(2,3- difluoropropoxy)pyridin-4- yl]methyl]-3-spiro[3.3]heptan- 2-ylurea | 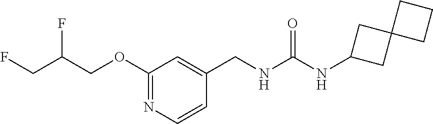 [SMILES: O=C(NCc1ccnc(OCC(F)CF)c1)NC1CC2(CCC2)C1] |
| 31 | 1-[[2- (difluoromethoxy)pyridin-4- yl]methyl]-3-(2,2- difluorospiro[2.5]octan-6- yl)urea | 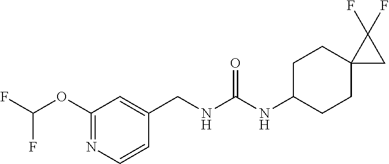 [SMILES: O=C(NCc1ccnc(OC(F)F)c1)NC1CCC2(CC1)CC2(F)F] |

In one embodiment, the invention provides a compound of formula (I), or a solvate or a pharmaceutically acceptable salt thereof, wherein R⁶ is a 7-11 membered oxa-spiro-heterocycloalkyl, which oxa-spiro-heterocycloalkyl is optionally substituted with one, two, or three substituents independently selected from halogen, C₁₋₆alkyl, hydroxy, and hydroxyC₁₋₆alkyl.

In some embodiments, compounds of this invention, wherein R⁶ is a 7-11 membered oxa-spiro-heterocycloalkyl are preferred.

In some embodiments, wherein R⁶ is a 7-11 membered oxa-spiro-heterocycloalkyl the compound of formula (I) is not:

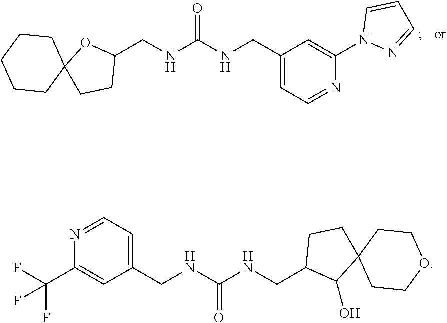 [SMILES: O=C(NCc1ccnc(-n2cccn2)c1)NCC1CCC2(CCCCC2)O1 ; O=C(NCc1ccnc(C(F)(F)F)c1)NCC1CCC2(CCOCC2)C1O]

In one preferred embodiment, the invention provides a compound of formula (I), or a solvate or a pharmaceutically acceptable salt thereof, wherein R¹ is a haloC₁₋₆alkyl or a haloC₁₋₆alkoxy and R⁶ is a 7-11 membered oxa-spiro-heterocycloalkyl as described herein.

In one embodiment, the invention provides a compound of formula (I), or a solvate or a pharmaceutically acceptable salt thereof, wherein R¹ is a 5-6 membered heteroaryl, a C₃₋₆cycloalkyl-C₁₋₆alkoxy, a haloC₁₋₆alkyl, a or a haloC₁₋₆alkoxy, R² and R³ are H, and R⁶ is a 7-11 membered oxa-spiro-heterocycloalkyl as described herein.

In one embodiment, the invention provides a compound of formula (I), or a solvate or a pharmaceutically acceptable salt thereof, wherein R¹ is a 5-6 membered heteroaryl, R² and R³ are H, and R⁶ is a 7-11 membered oxa-spiro-heterocycloalkyl as described herein.

In one embodiment, the invention provides a compound of formula (I), or a solvate or a pharmaceutically acceptable salt thereof, wherein R¹ is a a 5-6 membered heteroaryl, haloC₁₋₆alkyl or a haloC₁₋₆alkoxy, R² and R³ are H, and R⁶ is a 7-11 membered oxa-spiro-heterocycloalkyl as described herein.

In one embodiment, the invention provides a compound of formula (I), or a solvate or a pharmaceutically acceptable salt thereof, wherein R¹ is a 5-6 membered heteroaryl, haloC₁₋₆alkyl or a haloC₁₋₆alkoxy, R² and R³ are H, n is 0, and R⁶ is a 7-11 membered oxa-spiro-heterocycloalkyl as described herein.

In one preferred embodiment, the invention provides a compound of formula (I), or a solvate or a pharmaceutically acceptable salt thereof, wherein R¹, R², R³, R⁴, R⁵, and n are as described herein, and R⁶ is a 7-11 membered oxa-spiro-heterocycloalkyl; which oxa-spiro-heterocycloalkyl is unsubstituted.

In one preferred embodiment, the invention provides a compound of formula (I), or a solvate or a pharmaceutically acceptable salt thereof, wherein R¹, R², R³, R⁴, R⁵, and n are as described herein, and R⁶ is a 7-11 membered oxa-spiro-heterocycloalkyl, which oxa-spiro-heterocycloalkyl is selected from oxaspiro[3.4]octanyl, oxaspiro[4.5]decan-9-yl, and oxaspiro[4.4]nonanyl, which are optionally substituted with one or two substituents independently selected from halogen, C₁₋₆alkyl, hydroxy, and hydroxyC₁₋₆alkyl.

In one preferred embodiment, the invention provides a compound of formula (I), or a solvate or a pharmaceutically acceptable salt thereof, wherein R¹, R², R³, R⁴, R⁵, and n are as described herein, and R⁶ is selected from oxaspiro[3.4]octanyl, oxaspiro[4.5]decan-9-yl, and oxaspiro[4.4]nonanyl which are unsubstituted.

In one embodiment, the invention provides a compound of formula (I), or a solvate or a pharmaceutically acceptable salt thereof, wherein R¹, R², R³, R⁴, R⁵, and n are as described herein, and R⁶ is selected from oxaspiro[3.4]octanyl, oxaspiro[4.5]decan-9-yl, and oxaspiro[4.4]nonanyl which are optionally substituted with one or two substituents independently selected from halogen and hydroxyC₁₋₆alkyl.

In all of the herein embodiments of R⁶ as a 7-11 membered oxa-spiro-heterocycloalkyl, R¹, R², R³, R⁴, R⁵ and n are as described herein, i.e. including any combination of R¹, R², R³, R⁴, R⁵ and n are as described herein.

In some of the herein embodiments, R² is H and R³ is hydroxyC₁₋₆alkyl, in particular HOCH₂—.

In some preferred embodiments, n is 0.

In some particularly preferred embodiments, R¹ is haloC₁₋₆alkoxy, R² and R₃ are hydrogen, n is 0, and R⁶ is a 7-11 membered oxa-spiro-heterocycloalkyl optionally substituted as described herein.

In some particularly preferred embodiments, R¹ is haloC₁₋₆alkoxy, R² and R₃ are hydrogen, n is 0, and R⁶ is a 7-11 membered oxa-spiro-heterocycloalkyl which is unsubstituted.

In some preferred embodiments, R⁶ being the 7-11 membered oxa-spiro-heterocycloalkyl has the following structure:

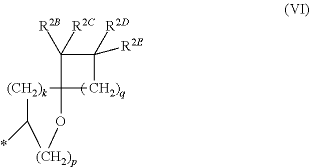

wherein

p is 1, or 2;

q is 1, 2, or 3;

k is 0, or 1

R^{2B}, R^{2C}; R^{2D}, and R^{2E} are independently selected from H, halogen, C₁₋₆alkyl, hydroxyl, and hydroxyC₁₋₆alkyl.

In some more preferred embodiments, R⁶ being the 7-11 membered oxa-spiro-heterocycloalkyl has the following structure:

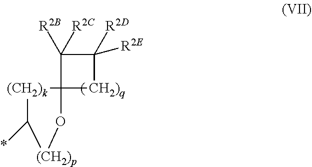

wherein
p is 1, or 2;
q is 1, 2, or 3;
k is 0, or 1
R^{2B}, R^{2C}; R^{2D}, and R^{2E} are independently selected from H and halogen.

In one particularly preferred embodiment hydroxyC₁₋₆alkyl is HOCH₂—.

In one particularly preferred embodiment, halogen is F.

In one particularly preferred embodiment, R^{2B}, R^{2C}; R^{2D}, and R^{2E} are all H.

In one particularly preferred embodiment, R^{2B}, R^{2C}; R^{2D}, and R^{2E} are all H, p is 2, q is 1, and k is 1.

In some embodiments, R⁶ being the 7-11 membered oxa-spiro-heterocycloalkyl has a structure selected from formulae (VI) or (VII) and R^{2B}, R^{2C}; R^{2D}, and R^{2E} are as described herein.

In some embodiments, R⁶ being the 7-11 membered oxa-spiro-heterocycloalkyl has a structure selected from formulae (VI) or (VII) and R^{2B}, R^{2C}; R^{2D}, and R^{2E} are as described herein, p is 2, q is 1, and k is 1.

In one embodiment, the invention provides a compound of formula (I), or a solvate or a pharmaceutically acceptable salt thereof, wherein R¹, R², R³, R⁴, R⁵ and n are as described herein, and R⁶ is a 7-11 membered oxa-spiro-heterocycloalkyl, and non-limiting examples are selected from Table 3.

TABLE 3

| | | |
|---|---|---|
| 1 | 1-(1- oxaspiro[4.4]nonan-3- yl)-3-[(2-pyrazol-1- ylpyridin-4- yl)methyl]urea | 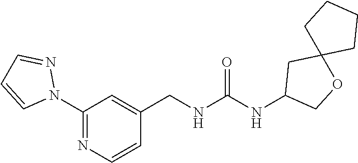 [SMILES: O=C(NCc1ccnc(-n2cccn2)c1)NC1COC2(CCCC2)C1] |
| 2 | 1-(6- oxaspiro[4.5]decan-9- yl)-3-[(2-pyrazol-1- ylpyridin-4- yl)methyl]urea | 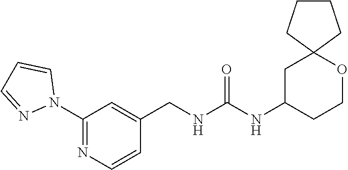 [SMILES: O=C(NCc1ccnc(-n2cccn2)c1)NC1CCOC2(CCCC2)C1] |
| 3 | 1-[[2- (difluoromethoxy)pyridin- 4-yl]methyl]-3-(1- oxaspiro[4.4]nonan-3- yl)urea | 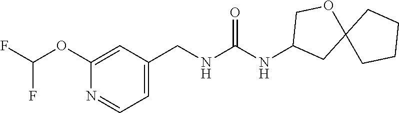 [SMILES: O=C(NCc1ccnc(OC(F)F)c1)NC1COC2(CCCC2)C1] |
| 14 | 1-[[2- (difluoromethoxy)pyridin- 4-yl]methyl]-3-(6- oxaspiro[4.5]decan-9- yl)urea | 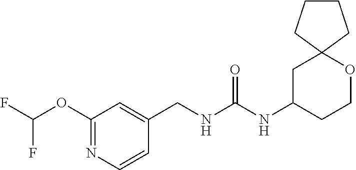 [SMILES: O=C(NCc1ccnc(OC(F)F)c1)NC1CCOC2(CCCC2)C1] |
| 17 | 1-[[2- (difluoromethoxy)pyridin- 4-yl]methyl]-3-(5- oxaspiro[3.5]nonan-8- yl)urea | 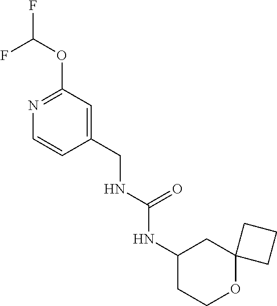 [SMILES: O=C(NCc1ccnc(OC(F)F)c1)NC1CCOC2(CCC2)C1] |
| 20 | 1-[[2- (difluoromethoxy)pyridin- 4-yl]methyl]-3-(1- oxaspiro[3.5]nonan-3- yl)urea | 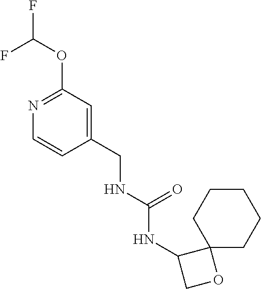 [SMILES: O=C(NCc1ccnc(OC(F)F)c1)NC1COC12CCCCC2] |
| 21 | 1-[[2- (difluoromethoxy)pyridin- 4-yl]methyl]-3-(4,4- difluoro-6- oxaspiro[4.5]decan-9- yl)urea | 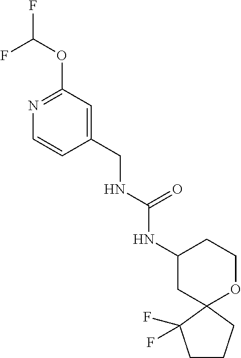 [SMILES: O=C(NCc1ccnc(OC(F)F)c1)NC1CCOC2(CCCC2(F)F)C1] |
| 22 | 1-[[2- (difluoromethoxy)pyridin- 4-yl]methyl]-3-(5- oxaspiro[3.4]octan-7- yl)urea | 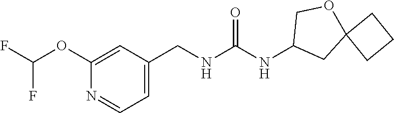 [SMILES: O=C(NCc1ccnc(OC(F)F)c1)NC1COC2(CCC2)C1] |
| 27 | 1-[[2- (difluoromethoxy)pyridin- 4-yl]methyl]-3-[(8R)- 5-oxaspiro[3.5]nonan-8- yl]urea | 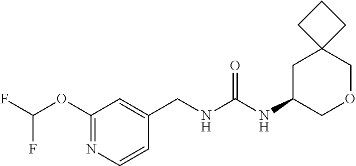 [SMILES: O=C(NCc1ccnc(OC(F)F)c1)N[C@@H]1COCC2(CCC2)C1] |
| 30 | 1-[[2- (difluoromethoxy)pyridin- 4-yl]methyl]-3-(1- oxaspiro[4.5]decan-3- yl)urea | 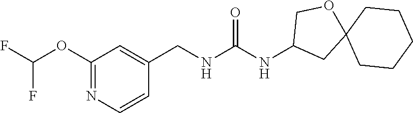 [SMILES: O=C(NCc1ccnc(OC(F)F)c1)NC1COC2(CCCCC2)C1] |
| 32 | 1-[[2- (difluoromethoxy)pyridin- 4-yl]methyl]-3-(5- oxaspiro[3.5]nonan-7- yl)urea | 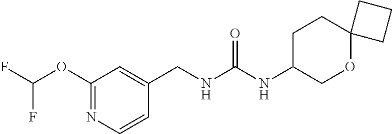 [SMILES: O=C(NCc1ccnc(OC(F)F)c1)NC1CCC2(CCC2)OC1] |

In some particularly preferred embodiments, R⁶ is

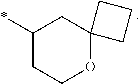 [SMILES: *C1CCOC2(CCC2)C1]

In some preferred embodiments, R⁶ is

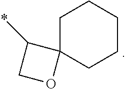 [SMILES: *C1COC12CCCCC2]

In some preferred embodiment, R⁶ is:

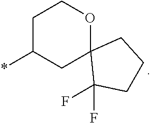 [SMILES: *C1CCOC2(CCCC2(F)F)C1]

In some preferred embodiments, R⁶ is:

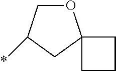 [SMILES: *C1COC2(CCC2)C1]

In some of the aforementioned embodiments, the O-atom is at a different position.

In some embodiments, the invention relates to a compound as described herein, wherein such compounds of formula (I) show Kv7.2 EC₅₀: <1 μM or <3 μM, as most preferred or preferred EC₅₀. In some embodiments such compounds show Kv7.2 EC₅₀: <3 μM.

In some embodiments of the invention, in the compounds of this invention one or more hydrogen atoms is (are) replaced by a deuterium. It has been surprisingly found that deuteration of the compounds of this invention offer the advantage of retaining the pharmacological profile of their hydrogen counterparts while positively impacting their metabolic outcome. Selective replacement of one or more hydrogen with deuterium, in the compounds of the present invention, improves the pharmaceutical profile of compounds of this invention, e.g. by reducing the amount of undesired metabolites when compared to its all hydrogen counterparts and by lowering rates of metabolism, and hence increasing half-life.

Methods for incorporation of deuterium into compounds are well established. Using metabolic studies established in the art, the compound of the present invention can be tested to identify sites for selective placement of a deuterium isotope, which isotope will not be metabolized or be metabolized at a lower rate. Moreover, these studies identify sites of metabolism as the location where a deuterium atom would be placed.

In one embodiment, the present invention provides a compound of this invention, wherein one or more of the hydrogen atoms are replaced with deuterium.

In some embodiments, if falling under the claims of this invention, the compounds of this invention do not include:

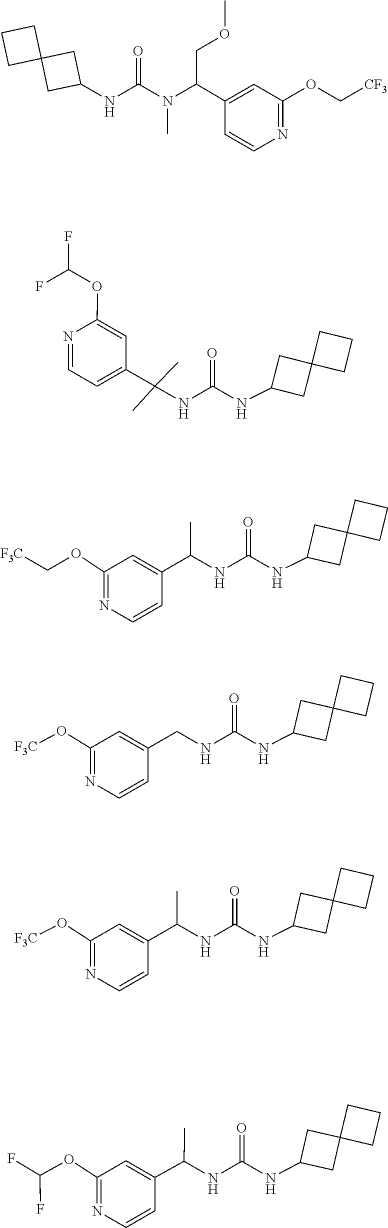 [SMILES: COCC(c1ccnc(OCC(F)(F)F)c1)N(C)C(=O)NC1CC2(CCC2)C1 ; CC(C)(NC(=O)NC1CC2(CCC2)C1)c1ccnc(OC(F)F)c1 ; CC(NC(=O)NC1CC2(CCC2)C1)c1ccnc(OCC(F)(F)F)c1 ; O=C(NCc1ccnc(OC(F)(F)F)c1)NC1CC2(CCC2)C1 ; CC(NC(=O)NC1CC2(CCC2)C1)c1ccnc(OC(F)(F)F)c1 ; CC(NC(=O)NC1CC2(CCC2)C1)c1ccnc(OC(F)F)c1]

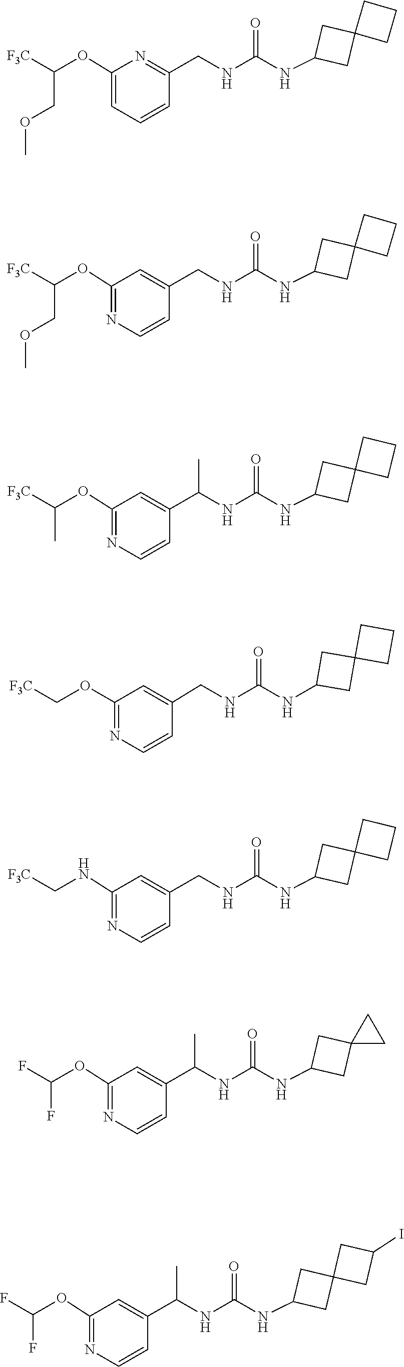

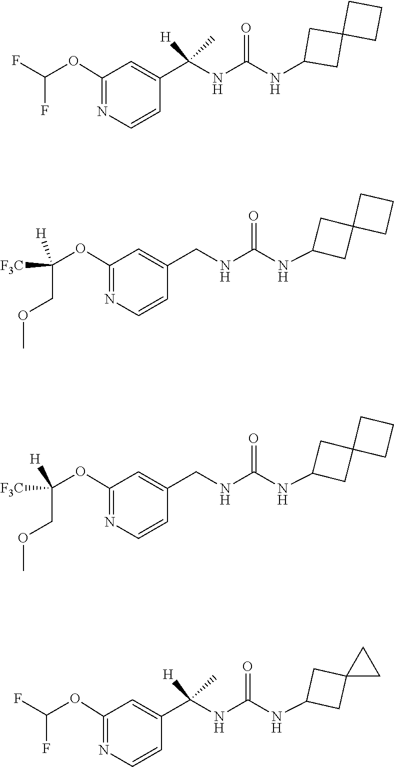 [SMILES: C[C@H](NC(=O)NC1CC2(CCC2)C1)c1ccnc(OC(F)F)c1 ; COC[C@H](Oc1cc(CNC(=O)NC2CC3(CCC3)C2)ccn1)C(F)(F)F ; COC[C@@H](Oc1cc(CNC(=O)NC2CC3(CCC3)C2)ccn1)C(F)(F)F ; C[C@H](NC(=O)NC1CC2(CC2)C1)c1ccnc(OC(F)F)c1]

The compounds of the invention have shown to be agents acting on Kv7.2 and are therefore useful for the treatment and/or prophylaxis of any of the diseases, disorders, or disabilities described herein. They are in particular useful for the therapeutic and/or prophylactic treatment of a disorder, disease, or disabilities associated with Kv7.2. More particularly, they are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus. The behavioral disorder is for example Attention Deficit Hyperactivity Disorder (ADHD). The mood disorder is for example depression. The neurodevelopment disorder is for example autism spectrum disorder (ASD) or a syndromic developmental disorder. The syndromic developmental disorder is for example Dup15q syndrome (Dup15q), Fragile X syndrome (FXS), and Angelman syndrome. The epilepsies are for example broad pediatric epilepsy, West syndrome, Ohtahara syndrome and epileptic encephalopathy. Neurodegenerative diseases are for example Alzheimer's disease, or motor neuron diseases.

The compounds of the invention are therefore useful Kv7.2 modulators that provide for favorable pharmacological properties, such as for example potency, selectivity, and metabolic stability.

### Pharmaceutical Compositions and Administration

In one embodiment, the present invention provides a pharmaceutical composition comprising a compound of this invention as described herein.

In one embodiment, the present invention provides a pharmaceutical composition comprising a compound of this invention as described herein and one or more pharmaceutically acceptable excipients.

In one embodiment, the present invention provides a pharmaceutical composition additionally comprising one or more pharmaceutical excipients selected form diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, and wet granulation agent.

In a particular embodiment, the invention provides pharmaceutical compositions as described above which are in particular useful for the therapeutic and/or prophylactic treatment of a disorder, disease, or disabilities associated with Kv7.2.

More particularly, the pharmaceutical compositions as described herein are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus.

The compounds of this invention can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of this invention can be processed with pharmaceutically inert, inorganic or organic adjuvants for the production of tablets, coated tablets, dragées and hard gelatin capsules. Lactose, corn starch, or derivatives thereof, talc, stearic acid or its salts etc. can be used, for example, as such adjuvants for tablets, dragées, or hard gelatin capsules.

Suitable adjuvants for soft gelatin capsules are, for example, vegetable oils, waxes, fats, semi-solid substances, or liquid polyols, etc.

Suitable adjuvants for the production of solutions and syrups are, for example, water, polyols, saccharose, invert sugar, or glucose, etc.

Suitable adjuvants for injection solutions are, for example, water, alcohols, polyols, glycerol, or vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Provided herein are pharmaceutical compositions comprising a compound of this invention. In some embodiments the pharmaceutical compositions comprise one or more pharmaceutically acceptable excipients. Conventional procedures for the selection and preparation of suitable pharmaceutical compositions are described in, for example, “Pharmaceuticals—The Science of Dosage Form Designs,” M. E. Aulton, Churchill Livingstone, 1988, which is hereby incorporated by reference in its entirety.

Further provided is a process for the preparation of a pharmaceutical composition, comprising combining one or more compounds of this invention.

Further provided is a process for the preparation of a pharmaceutical composition, comprising combining one or more compounds of this invention with one or more pharmaceutically acceptable excipients. Pharmaceutical compositions may be prepared, for example, according to conventional dissolution, mixing, granulating, or coating methods, or combinations thereof. Such pharmaceutically acceptable excipients may include, for example, sugars (e.g., lactose, glucose, sucrose); starches (e.g., corn starch, potato starch); cellulose and its derivatives (e.g., sodium carboxymethyl cellulose, ethyl cellulose, cellulose acetate); powdered tragacanth; malt; gelatin; talc; cocoa butter and suppository waxes; oils (e.g., peanut oil, cottonseed oil, safflower oil, sesame oil, olive oil, corn oil, soybean oil); glycols (e.g., propylene glycol); polyethylene glycols (PEG); esters (e.g., ethyl oleate, ethyl laurate); agar; buffering agents (e.g., magnesium hydroxide, aluminum hydroxide); alginic acid; pyrogen-free water; isotonic saline; Ringer's solution; ethyl alcohol; phosphate buffer solutions; non-toxic compatible lubricants (e.g., sodium lauryl sulfate, magnesium stearate); coloring agents; releasing agents; coating agents; sweetening; and flavoring and perfuming agents. Preservatives and antioxidants can also be present in the pharmaceutical composition, according to the judgment of the formulator.

Depending on the intended mode of administration, the disclosed pharmaceutical compositions can be in solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. These modes may include systemic or local administration such as oral, nasal, parenteral (as by intravenous (both bolus and infusion), intramuscular, or subcutaneous injection), transdermal, vaginal, buccal, rectal, or topical (as by powders, ointments, or drops) administration modes. These modes may also include intracisternally, intraperitoneally, as an oral or nasal spray, or as a liquid aerosol or dry powder pharmaceutical composition for inhalation. In some embodiments, the pharmaceutical composition provided herein comprises one or more disclosed compounds, tautomers thereof, and/or pharmaceutically acceptable salts thereof, and is for oral administration. In other embodiments, the pharmaceutical composition is for intravenous administration.

Solid dosage forms for oral administration may include capsules (e.g., soft and hard-filled gelatin capsules), tablets, pills, powders, and granules. Solid dosage forms may be prepared, in some embodiments, with one or more coatings and/or shells such as release controlling coatings, for example enteric coatings. Solid dosage forms may be formulated to release the one or more disclosed compounds (or solvate, tautomer, or pharmaceutically acceptable salt thereof) only, or mostly, or preferentially in a certain part of the gastrointestinal tract, optionally in a delayed manner. Solid dosage forms may also include, for example, micro-encapsulated forms.

Liquid dosage forms for oral administration may include, for example, pharmaceutically acceptable emulsions, microemulsions, solutions, suspensions, syrups, and elixirs. Such liquid compositions may include, for example, a pharmaceutically acceptable excipient such as water or other solvents, solubilizing agents, emulsifiers, oils, polyethylene glycols and fatty acid esters, adjuvants, sweetening agents, flavoring agents, or perfuming agents, or any combinations thereof.

Injectible pharmaceutical compositions include, for example, sterile injectable aqueous compositions (e.g., solutions, suspensions, or emulsions), or oleaginous suspensions.

Injectable pharmaceutical compositions may comprise, in some embodiments, one or more solvents and/or diluents, such as water, Ringer's solution, U.S.P. and isotonic sodium chloride solution, sterile fixed oils, fatty acid, or any combinations thereof. In some embodiments, an injectible pharmaceutical composition may be prepared as a lyophilized powder, for example a lyophilized powder that is to be mixed with a liquid diluent prior to injection.

In some embodiments, it may be desirable to prolong the effect of one or more compounds as disclosed herein, or pharmaceutically acceptable salt thereof, from administration through subcutaneous or intramuscular injection. Such delay may be accomplished, for example, through the use of a liquid suspension of crystalline or amorphous material with poor water solubility; or dissolving or suspending the compound, or solvate, tautomer, or pharmaceutically acceptable salt thereof, in an oil vehicle; or through an injectable depot form comprising microencapsule matrixes comprising one or more biodegradable polymers.

Pharmaceutical compositions for rectal or vaginal administration may include suppositories that can be prepared, for example using a suitable non-irritating excipient such as cocoa butter, polyethylene glycol, or a suppository wax; or using a fatty emulsion or suspension.

Dosage forms for topical or transdermal administration may include, for example, ointments, pastes, creams, lotions, gels, powders, solutions, sprays, inhalants, or patches. Ophthalmic pharmaceutical compositions and ear drops may also be prepared.

The pharmaceutical compositions provided herein may be packaged in unit-dose or multidose containers, for example sealed ampoules or vials, and may be stored in a freeze-dried (lyophilized) condition requiring only the addition of the sterile liquid excipient (e.g., diluent, carrier, for example water) for injection immediately prior to use. Extemporaneous injection solutions and suspensions may be prepared from sterile powders, granules, or tablets of the kind described herein. Unit dosage formulations include those containing a daily dose or unit daily sub-dose, or an appropriate fraction thereof, of the active ingredient.

The subject matter further provides veterinary compositions comprising at least one active ingredient as herein defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Medical Use

The compounds of this invention or pharmaceutical compositions comprising the same, as described herein, may be useful as pharmaceuticals for the therapeutic and/or prophylactic treatment of a disorder, disease or disability associated with Kv7.2 in a subject in need thereof.

In one embodiment, the present invention provides a method for the therapeutic and/or prophylactic treatment of a disorder, disease, or disability associated with Kv7.2 in a subject in need thereof, comprising administering an effective amount of compounds of this invention or pharmaceutical compositions thereof, as described herein.

In one embodiment, the present invention provides a method for the therapeutic and/or prophylactic treatment of a disorder, disease, or disability associated with Kv7.2 in a subject in need thereof, comprising administering an effective amount of the compounds of this invention, or pharmaceutical compositions comprising the same, as described herein, wherein the pharmaceutical composition further comprises one or more pharmaceutically acceptable excipients.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

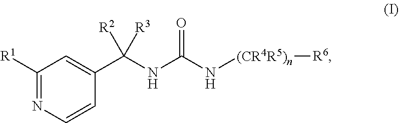

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

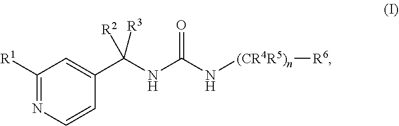

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

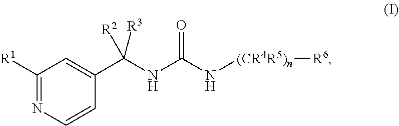

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the present invention provides a method for the therapeutic and/or prophylactic treatment of a disorder, disease, or disability associated with Kv7.2 in a subject in need thereof, comprising a compound of formula (I), or a solvate or a pharmaceutically acceptable salt thereof:

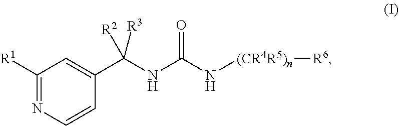

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the present invention provides a method for the therapeutic and/or prophylactic treatment of a disorder, disease, or disability associated with Kv7.2 in a subject in need thereof, comprising a compound of formulae (I)-(VII), or a solvate or a pharmaceutically acceptable salt thereof, any exemplified compound, any embodiment, or combinations of embodiments, as described herein.

In one embodiment, the present invention provides a method for the therapeutic and/or prophylactic treatment of a disorder, disease, or disability associated with Kv7.2 in a subject in need thereof, comprising a compound selected from any list of compounds as described herein, or solvates or pharmaceutically acceptable salts thereof.

In one embodiment, the present invention provides a method for the therapeutic and/or prophylactic treatment of a disorder, disease, or disability associated with Kv7.2 in a subject in need thereof, comprising a compound selected from Table 1, 2, and 3, or a solvate or a pharmaceutically acceptable salt thereof.

In one embodiment, the present invention provides a method for the therapeutic and/or prophylactic treatment of a disorder, disease, or disability associated with Kv7.2 in a subject in need thereof, comprising a compound of formulae (I)-(VII), or a solvate or a pharmaceutically acceptable salt thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the above method for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the above method for the therapeutic and/or prophylactic treatment of a disorder, disease, or disability associated with Kv7.2 in a subject in need thereof, wherein the mood disorder is depression.

In one embodiment, the present invention provides the above method for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the above method for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the above method for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the above method for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a method for the therapeutic and/or prophylactic treatment of a disorder, disease, or disability associated with Kv7.2 in a subject in need thereof, comprising administering an effective amount of pharmaceutical compositions as described herein.

Further provided herein is a compound, a solvate, a pharmaceutically acceptable salt or a pharmaceutical composition thereof, as described herein, for use as therapeutically active substance.

In one embodiment, the present invention provides the use of a compound of formula (I), or a solvate or a pharmaceutically acceptable salt thereof:

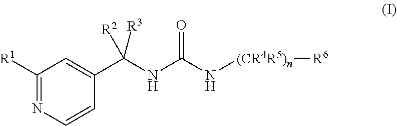

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
as therapeutically active substance.

In one embodiment, the present invention provides the use of a compound of formula (I), or a solvate or a pharmaceutically acceptable salt thereof:

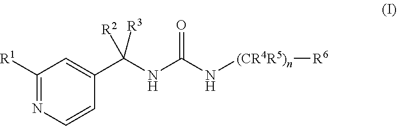

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
as therapeutically active substance.

In one embodiment, the present invention provides the use of a compound of formula (I), or a solvate or a pharmaceutically acceptable salt thereof:

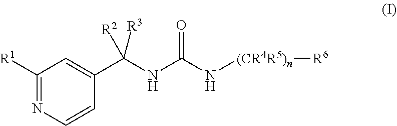

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
as therapeutically active substance.

In one embodiment, the present invention provides the use of a compound of formulae (I)-(VII), or a solvate or a pharmaceutically acceptable salt thereof, any exemplified compound, any embodiment, or combinations of embodiments, as described herein, as therapeutically active substance.

In one embodiment, the present invention provides the use of a compound selected from Table 1, 2, and 3, or a solvate or a pharmaceutically acceptable salt thereof, as therapeutically active substance.

In one embodiment, the present invention provides the use of a compound selected from any list of compounds described herein, or a solvate or a pharmaceutically acceptable salt thereof, as therapeutically active substance.

Further provided herein is a compound, a solvate, a pharmaceutically acceptable salt, or a pharmaceutical composition thereof, for use in the therapeutic and/or prophylactic treatment of a disorder, disease, or disability associated with Kv7.2 in a subject in need thereof.

In one embodiment, the present invention provides a compound of formula (I), or a solvate or a pharmaceutically acceptable salt thereof:

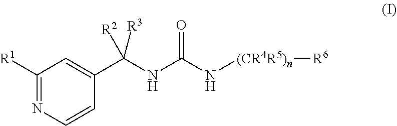

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
for use in the therapeutic and/or prophylactic treatment of a disorder, disease, or disability associated with Kv7.2 in a subject in need thereof.

In one embodiment, the present invention provides a compound of formula (I), or a solvate or a pharmaceutically acceptable salt thereof:

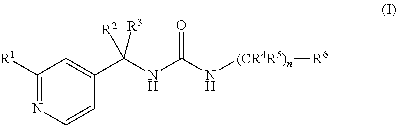

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
for use in the therapeutic and/or prophylactic treatment of a disorder, disease, or disability associated with Kv7.2 in a subject in need thereof.

In one embodiment, the present invention provides a compound of formula (I), or a solvate or a pharmaceutically acceptable salt thereof:

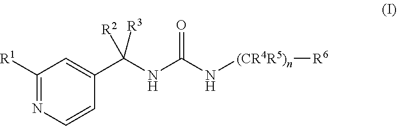

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
for use in the therapeutic and/or prophylactic treatment of a disorder, disease, or disability associated with Kv7.2 in a subject in need thereof.

In one embodiment, the present invention provides a compound of formulae (I)-(VII), or a solvate or a pharmaceutically acceptable salt thereof, any exemplified compound, any embodiment, or combinations of embodiments, as described herein, for use in the therapeutic and/or prophylactic treatment of a disorder, disease, or disability associated with Kv7.2 in a subject in need thereof.

In one embodiment, the present invention provides a compound selected from Table 1, 2, and 3, or a solvate or a pharmaceutically acceptable salt thereof, for use in the therapeutic and/or prophylactic treatment of a disorder, disease, or disability associated with Kv7.2 in a subject in need thereof.

In one embodiment, the present invention provides a compound selected from any list of compounds of this invention, for use in the therapeutic and/or prophylactic treatment of a disorder, disease, or disability associated with Kv7.2 in a subject in need thereof.

In one embodiment, the present invention provides a compound of this invention for use in the therapeutic and/or prophylactic treatment of a disorder, disease, or disability associated with Kv7.2 in a subject in need thereof, wherein such disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a compound of this invention for use in the therapeutic and/or prophylactic treatment of a disorder, disease, or disability associated with Kv7.2 in a subject in need thereof, wherein the mood disorder is depression.

In one embodiment, the present invention provides a compound of this invention, for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a compound of this invention for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

Further provided herein is the use of a compound of this invention in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

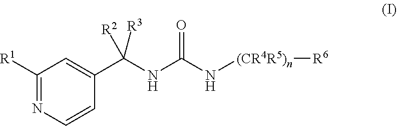

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

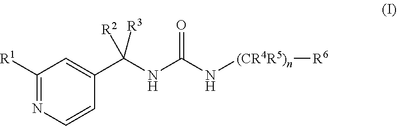

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

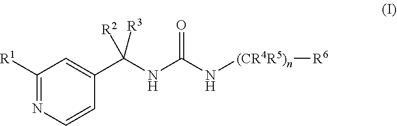

wherein
R¹ is selected from a 5-6 membered heteroaryl, haloC₁₋₆alkoxy cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
in the therapeutic and/or prophylactic treatment of a disorder, disease, or disability associated with Kv7.2 in a subject in need thereof.

In one embodiment, the present invention provides the use of a compound of formulae (I)-(VII), or solvate or a pharmaceutically acceptable salt thereof, any exemplified compound, any embodiment, or combinations of embodiments, as described herein, as described herein, in the therapeutic and/or prophylactic treatment of a disorder, disease, or disability associated with Kv7.2 in a subject in need thereof.

In one embodiment, the present invention provides the use of a compound selected from Table 1, 2, and 3, or solvate or a pharmaceutically acceptable salt thereof, in the therapeutic and/or prophylactic treatment of a disorder, disease, or disability associated with Kv7.2 in a subject in need thereof.

In one embodiment, the invention provides the use a compound selected from any list of compounds described herein, or a solvate or a pharmaceutically acceptable salt thereof, in the therapeutic and/or prophylactic treatment of a disorder, disease, or disability associated with Kv7.2 in a subject in need thereof.

In one embodiment, the invention provides for the use of a compound of this invention in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the invention provides for the use of a compound of this invention in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the invention provides for the use of a compound of this invention in the therapeutic and/or prophylactic treatment of a disorder, disease, or disability associated with Kv7.2 in a subject in need thereof, wherein the mood disorder is depression.

In one embodiment, the invention provides for the use of a compound of this invention in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the invention provides for the use of a compound of this invention in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the invention provides for the use of a compound of this invention in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the invention provides for the use of a compound of this invention in the therapeutic and/or prophylactic treatment of a disorder, disease, or disability associated with Kv7.2 in a subject in need thereof, the neurodegenerative diseases are selected from Alzheimer's disease and motor neuron diseases.

Further provided herein is the use of a a compound of this invention or a pharmaceutical composition comprising the same, for the manufacture of a medicament for the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

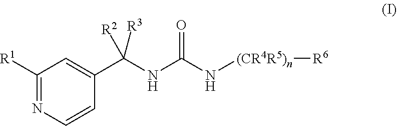

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl,
for the manufacture of a medicament for use in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

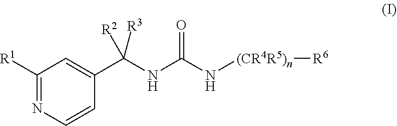

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclooxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl,
for the manufacture of a medicament for use in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

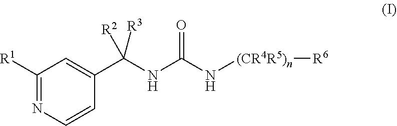

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl,
for the manufacture of a medicament for use in the therapeutic and/or prophylactic treatment of a disorder, disease, or disability associated with Kv7.2 in a subject in need thereof.

In one embodiment, the present invention provides the use of a compound of formulae (I)-(VII), or a solvate or a pharmaceutically acceptable salt thereof, any exemplified compound, any embodiment, or combinations of embodiments, as described herein, for the manufacture of a medicament for use in the therapeutic and/or prophylactic treatment of a disorder, disease, or disability associated with Kv7.2 in a subject in need thereof.

In one embodiment, the present invention provides the use a compound selected from Table 1, 2, and 3, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound selected from any list of compounds described herein, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the mood disorder is depression.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the use of a compound of this invention for the manufacture of a medicament for use in the therapeutic and/or prophylactic treatment of a disorder, disease, or disability associated with Kv7.2 in a subject in need thereof, the neurodegenerative diseases are selected from Alzheimer's disease and motor neuron diseases.

Further provided a pharmaceutical composition comprising a compound as described herein, or solvate or a pharmaceutically acceptable salt thereof.

Further provided a pharmaceutical composition comprising a compound as described herein, or solvate or a pharmaceutically acceptable salt thereof, wherein the pharmaceutical composition further comprises one or more pharmaceutically acceptable excipients.

In one particularly preferred embodiment, the invention provides a pharmaceutical composition comprising a compound of formula (I), or a solvate or a pharmaceutically acceptable salt thereof:

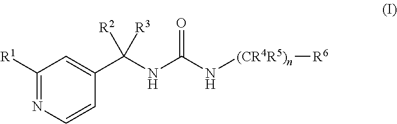

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the invention provides a pharmaceutical composition comprising a compound of formula (I), or a solvate or a pharmaceutically acceptable salt thereof:

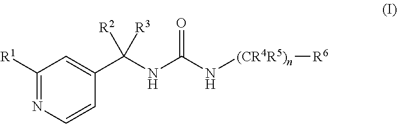

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the invention provides a pharmaceutical composition comprising a compound of formula (I), or a solvate or a pharmaceutically acceptable salt thereof:

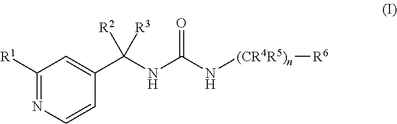

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the invention provides a pharmaceutical composition comprising a compound of formula (I), or a solvate or a pharmaceutically acceptable salt thereof:

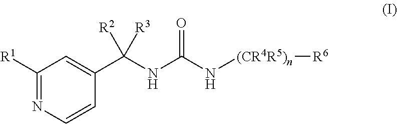

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one particularly preferred embodiment, the invention provides a pharmaceutical composition comprising a compound of formula (I), or a solvate or a pharmaceutically acceptable salt thereof:

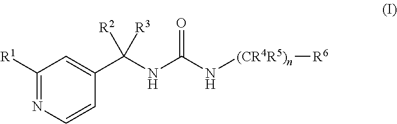

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloakylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one particularly preferred embodiment, the invention provides a pharmaceutical composition comprising a compound of formula (I), or a solvate or a pharmaceutically acceptable salt thereof:

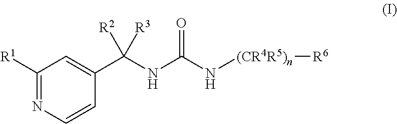

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

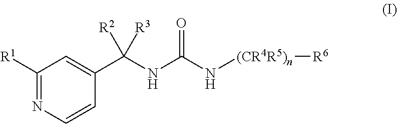

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the present invention provides a pharmaceutical composition comprising a compound of formula (I), or a solvate or a pharmaceutically acceptable salt thereof:

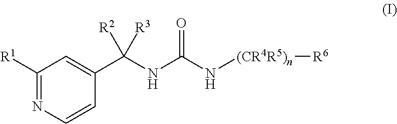

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and ₅heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the present invention provides a pharmaceutical composition comprising a compound of formula (I), or a solvate or a pharmaceutically acceptable salt thereof:

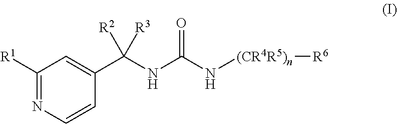

wherein

R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the present invention provides a pharmaceutical composition comprising a compound of formula (I), or a solvate or a pharmaceutically acceptable salt thereof:

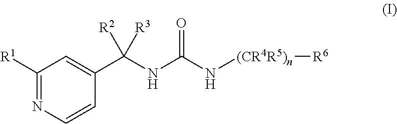

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

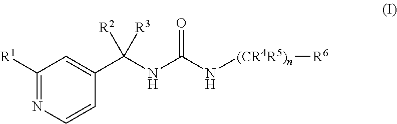

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

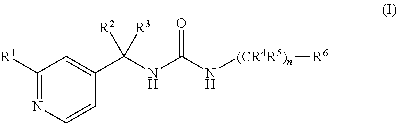

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl
and wherein the pharmaceutical composition further comprises one or more pharmaceutically acceptable excipients.

In one particularly preferred embodiment, the present invention provides a pharmaceutical composition comprising a compound of formulae (I)-(VII), or a solvate or a pharmaceutically acceptable salt thereof, as described herein, for use in the therapeutic and/or prophylactic treatment of a disorder, disease, or disability associated with Kv7.2 in a subject in need thereof.

In one particularly preferred embodiment, the present invention provides a pharmaceutical composition comprising a compound of formulae (I)-(VII), or a solvate or a pharmaceutically acceptable salt thereof, as described herein for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one particularly preferred embodiment, the present invention provides a pharmaceutical composition comprising a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound of formulae (I)-(VII), or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, 2, and 3, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound of this invention for use in a method of the therapeutic and/or prophylactic treatment of disorder, disease or disability associated with Kv7.2 in a subject in need thereof.

Further provided a pharmaceutical composition comprising a compound of this invention, and one or more pharmaceutically acceptable excipients, for use in a method of the therapeutic and/or prophylactic treatment of disorder, disease or disability associated with Kv7.2 in a subject in need thereof.

In one embodiment, the invention provides a pharmaceutical composition for use in a method of treatment comprising a compound of formula (I), or a solvate or a pharmaceutically acceptable salt thereof:

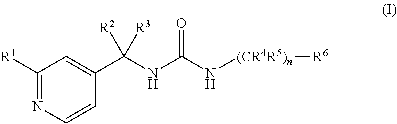

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one particularly preferred embodiment, the present invention provides a pharmaceutical composition comprising a compound of formula (I), or a solvate or a pharmaceutically acceptable salt thereof:

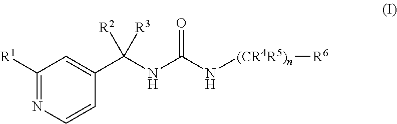

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl
and wherein the pharmaceutical composition further comprises one or more pharmaceutically acceptable excipients.

In one particularly preferred embodiment, the present invention provides a pharmaceutical composition comprising a compound of formula (I), or a solvate or a pharmaceutically acceptable salt thereof:

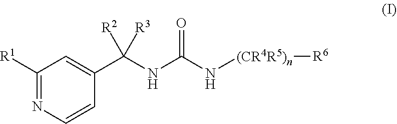

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl
and wherein the pharmaceutical composition further comprises one or more pharmaceutically acceptable excipients.

In one embodiment, the invention provides a pharmaceutical composition for use in a method of treatment comprising a compound of formula (I), or a solvate or a pharmaceutically acceptable salt thereof:

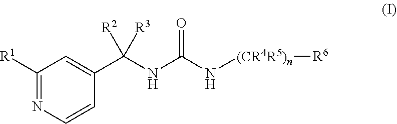

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the invention provides a pharmaceutical composition for use in a method of treatment comprising a compound of formula (I), or a solvate or a pharmaceutically acceptable salt thereof:

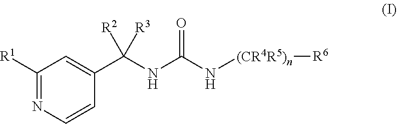

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the invention provides a pharmaceutical composition for use in a method of treatment comprising a compound of formula (I), or a solvate or a pharmaceutically acceptable salt thereof:

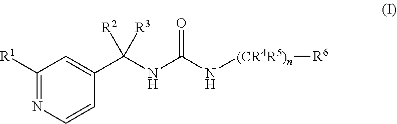

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

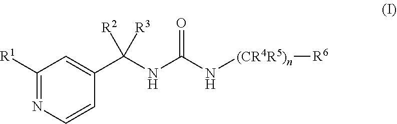

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in a method of treatment comprising a compound of formula (I), or a solvate or a pharmaceutically acceptable salt thereof:

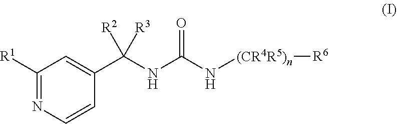

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in a method of treatment comprising a compound of formula (I), or a solvate or a pharmaceutically acceptable salt thereof:

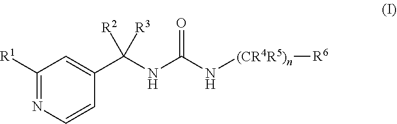

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in a method of treatment comprising a compound of formula (I), or a solvate or a pharmaceutically acceptable salt thereof:

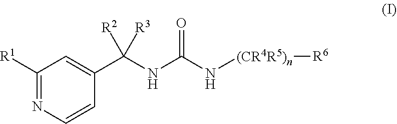

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

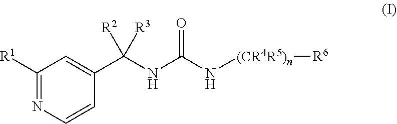

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in a method of treatment comprising a compound of formula (I), or a solvate or a pharmaceutically acceptable salt thereof:

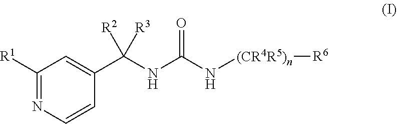

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition for use in a method of treatment comprises a compound of this invention for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the mood disorder is depression.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I)-(VII), or a solvate or a pharmaceutically acceptable salt thereof, any exemplified compound, any embodiment, or combinations of embodiments, as described herein.

In one embodiment, the present invention provides a pharmaceutical composition as described above comprising a compound selected from Table 1, 2, and 3, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease, or disability associated with Kv7.2 in a subject in need thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease or disability associated with Kv7.2 in a subject in need thereof, wherein the pharmaceutical further comprises one or more pharmaceutically acceptable excipients.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

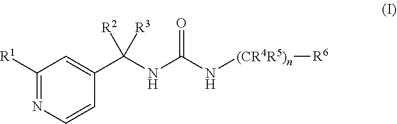

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

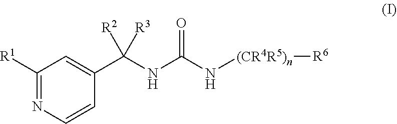

wherein

R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

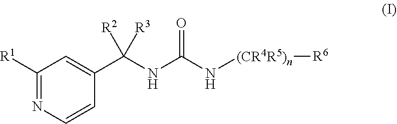

wherein
R¹ is selected from a 5-6 membered heteroaryl, haloC₁₋₆alkoxy, cyano, haloC₁₋₆alkyl, halogen, C₁₋₆ alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

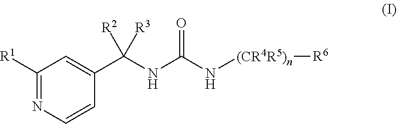

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
and wherein the pharmaceutical composition further comprises one or more pharmaceutically acceptable excipients.

In one embodiment, the invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

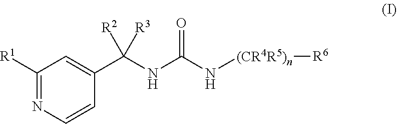

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalklyalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

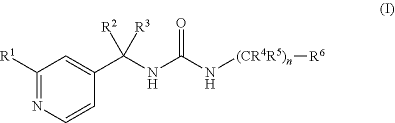

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

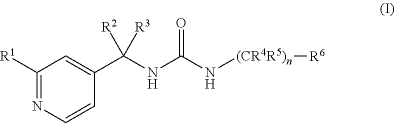

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

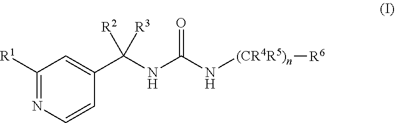

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

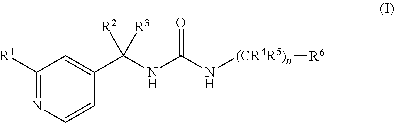

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

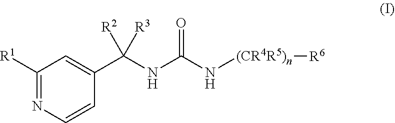

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

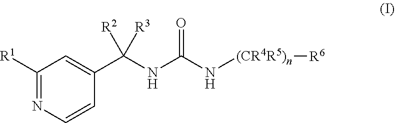

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

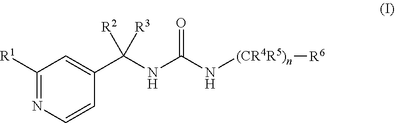

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

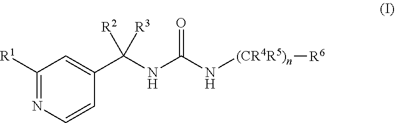

wherein

R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclooxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

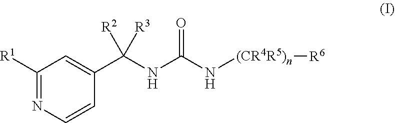

wherein
R¹ is selected from a 5-6 membered heteroaryl, haloC₁₋₆alkoxy, cyano, haloC₁₋₆alkyl, halogen, C₁₋₆ alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

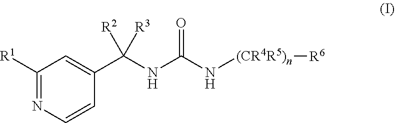

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

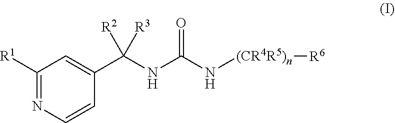

wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

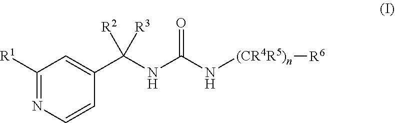

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl, hydroxy, and hydroxyC₁₋₆alkyl;
and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition a compound of this invention for use in manufacture of a medicament for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I)-(VII), or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, 2, and 3, or a solvate or a pharmaceutically acceptable salt thereof.

In one embodiment, the pharmaceutical composition as described above comprises a compound selected from any list of compounds described herein, or a solvate or a pharmaceutically acceptable salt thereof.

In some embodiments, the invention relates to any medical use of a compound as described herein, wherein R¹ is selected from 5 membered heteroaryl, haloC₁₋₆alkoxy, and haloC₁₋₆alkyl.

In some embodiments, the invention relates to any medical use of a compound as described herein, wherein R¹ is unsubstituted pyrazolyl.

In some embodiments, the invention relates to any medical use of a compound as described herein, wherein R¹ is C₃₋₅cycloalkyl-C₁₋₆alkoxy.

In some embodiments, the invention relates to any medical use of a compound as described herein, wherein R¹ is haloC₁₋₆alkoxy selected from CHF₂O—, CH₃CF₂O—, CH₃CFHCH₂O—, and CF₃CH₂O—.

In some embodiments, the invention relates to any medical use of a compound as described herein, wherein R¹ is haloC₁₋₆alkyl selected from CHF₂—, CF₃—, FCH₂CFHCH₂—, and CF₃CH₂—.

In some embodiments, the invention relates to any medical use of a compound as described herein, wherein (i) both R² and R³ are H, or (ii) R² is HOCH₂— and R³ is H.

In some embodiments, the invention relates to any medical use of a compound as described herein, wherein n is 0.

In some embodiments, the invention relates to any medical use of a compound according to a compound as described herein, wherein one or both of R⁴ and R⁵ are halogen.

In some embodiments, the invention relates to any medical use of a compound as described herein, wherein both of R⁴ and R⁵ are halogen.

In some embodiments, the invention relates to any medical use of a compound as described herein, wherein halogen is F—.

In some embodiments, the invention relates to any medical use of a compound as described herein, wherein R⁶ is substituted with one or two substituents independently selected from F— and hydroxyC₁₋₆alkyl.

In some embodiments, the invention relates to any medical use of a compound as described herein, wherein R⁶ is selected from a group consisting of:

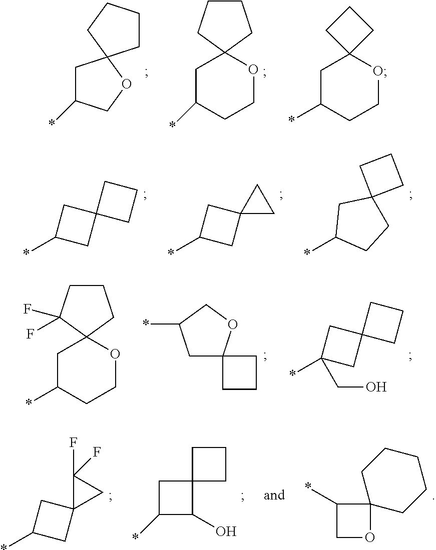

In some embodiments, the invention relates to any medical use of a compound as described herein, wherein R⁶ is selected from a group consisting of:

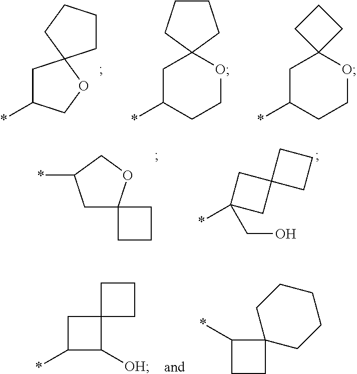

In some embodiments, the invention relates to any medical use of a compound as described herein, wherein R⁶ is selected from a group consisting of:

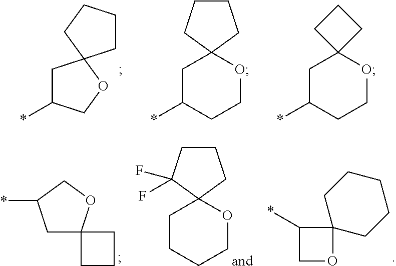

In preferred embodiments of those shown above for R⁶, n is 0.

In some embodiments, the invention relates to any medical use of a compound as described herein, wherein R⁶ is selected from a group consisting of:

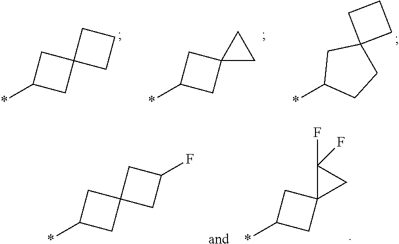

In some embodiments, the invention relates to any medical use of a compound as described herein, wherein R¹ is CHF₂O—, R² and are R³ are H, n is 0 and R⁶ is selected from the group consisting of:

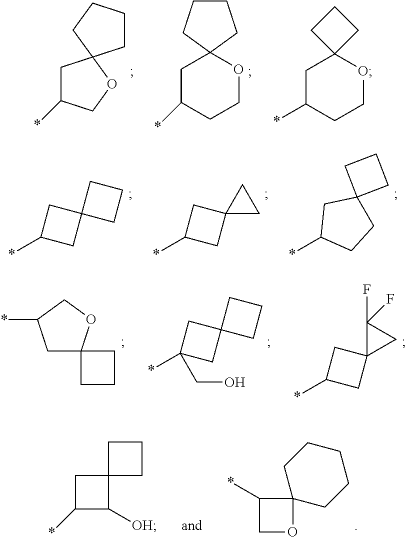

In some embodiments, the invention relates to any medical use of a compound as described herein, wherein R¹ is CHF₂O—, R² and are R³ are H, n is 0 and R⁶ is selected from the group consisting of:

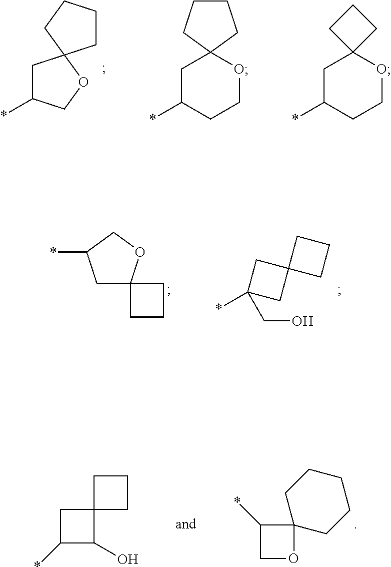

In some particularly preferred embodiments, the invention relates to any medical use of a compound as described herein, wherein R¹ is CHF₂O—, R² and are R³ are H, n is 0 and R⁶ is selected from the group consisting of:

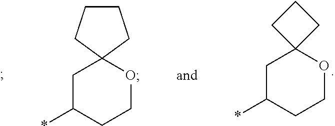 [SMILES: ";" ; *C1CCOC2(CCCC2)C1 ; "and" ; *C1CCOC2(CCC2)C1 ; "."]

In some embodiments, the invention relates to to any medical use of a compound as described herein, selected from the following list:

| | | |
|---|---|---|
| 1 | 1-(1- oxaspiro[4.4]nonan- 3-yl)-3-[(2- pyrazol-1- ylpyridin-4- yl)methyl]urea | 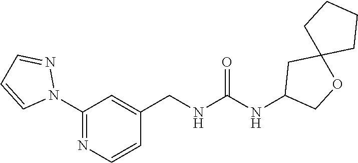 [SMILES: O=C(NCc1ccnc(-n2cccn2)c1)NC1COC2(CCCC2)C1] |
| 2 | 1-(6- oxaspiro[4.5]decan- 9-yl)-3-[(2- pyrazol-1- ylpyridin-4- yl)methyl]urea | 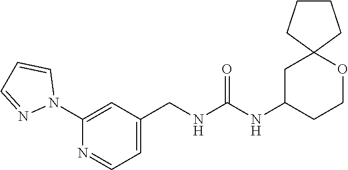 [SMILES: O=C(NCc1ccnc(-n2cccn2)c1)NC1CCOC2(CCCC2)C1] |
| 3 | 1-[[2- (difluoromethoxy) pyridin-4- yl]methyl]-3-(1- oxaspiro[4.4]nonan- 3-yl)urea | 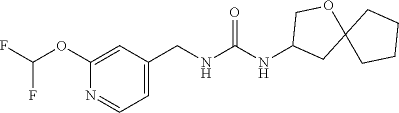 [SMILES: O=C(NCc1ccnc(OC(F)F)c1)NC1COC2(CCCC2)C1] |
| 4 | 1-[[2- (difluoromethoxy) pyridin-4- yl]methyl]-3- spiro[3.3]heptan- 2-ylurea | 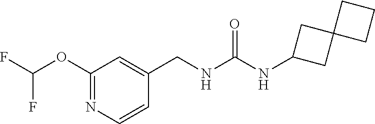 [SMILES: O=C(NCc1ccnc(OC(F)F)c1)NC1CC2(CCC2)C1] |
| 5 | 1-[[2-(2,2- difluoropropoxy) pyridin-4- yl]methyl]-3- spiro[3.3]heptan- 2-ylurea | 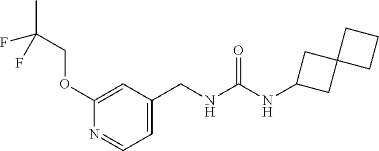 [SMILES: CC(F)(F)COc1cc(CNC(=O)NC2CC3(CCC3)C2)ccn1] |
| 6 | 1-[[2-(2- fluoropropoxy) pyridin-4-yl]methyl]- 3- spiro[3.3]eptan- 2-ylurea | 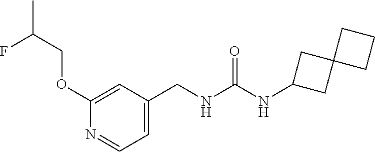 [SMILES: CC(F)COc1cc(CNC(=O)NC2CC3(CCC3)C2)ccn1] |
| 7 | 1-[[2- (cyclopropylmethoxy) pyridin-4- yl]methyl]-3- spiro[3.3]heptan- 2-ylurea | 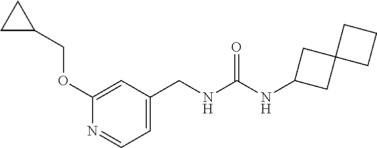 [SMILES: O=C(NCc1ccnc(OCC2CC2)c1)NC1CC2(CCC2)C1] |
| 8 | 1-[[2- (difluoromethoxy) pyridin-4- yl]methyl]-3- spiro[2.3]hexan-5- ylurea | 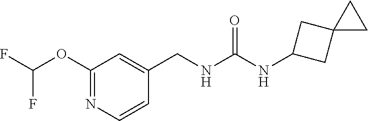 [SMILES: O=C(NCc1ccnc(OC(F)F)c1)NC1CC2(CC2)C1] |
| 9 | 1-[[2-(1,1- difluoroethyl)pyridin- 4-yl]methyl]- 3- spiro[3.3]heptan- 2-ylurea | 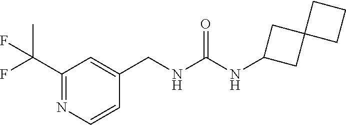 [SMILES: CC(F)(F)c1cc(CNC(=O)NC2CC3(CCC3)C2)ccn1] |
| 10 | 1- spiro[3.3]heptan- 2-yl-3-[[2- (trifluoromethyl) pyridin-4- yl]methyl]urea | 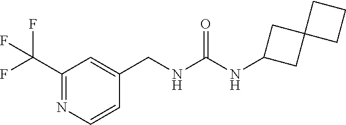 [SMILES: O=C(NCc1ccnc(C(F)(F)F)c1)NC1CC2(CCC2)C1] |
| 11 | 1-[2- (difluoromethoxy) pyridin-4- yl]methyl]-3- spiro[3.4]octan-7- ylurea | 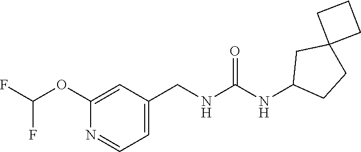 [SMILES: O=C(NCc1ccnc(OC(F)F)c1)NC1CCC2(CCC2)C1] |
| 12 | 1 -spiro[2.3]hexan- 5-yl-3-[[2-(2,2,2- trifluoroethoxy) pyridin-4- yl]methyl]urea | 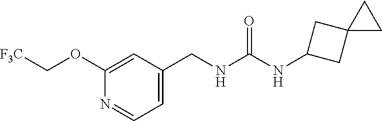 [SMILES: O=C(NCc1ccnc(OCC(F)(F)F)c1)NC1CC2(CC2)C1] |
| 13 | 1-[2-hydroxy-1- [2-(2,2,2- trifluoroethoxy) pyridin-4-yl]ethyl]- 3- spiro[3.3]heptan- 2-ylurea | 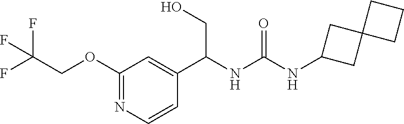 [SMILES: O=C(NC1CC2(CCC2)C1)NC(CO)c1ccnc(OCC(F)(F)F)c1] |
| 14 | 1-[[2- (difluoromethoxy) pyridin-4- yl]methyl]-3-(6- oxaspiro[4.5]decan- 9-yl)urea | 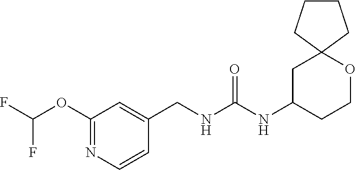 [SMILES: O=C(NCc1ccnc(OC(F)F)c1)NC1CCOC2(CCCC2)C1] |
| 15 | 1-[[2- (difluoromethoxy) pyridin-4- yl]methyl]-3-(2,2- difluorospiro[2.3] hexan-5-yl)urea | 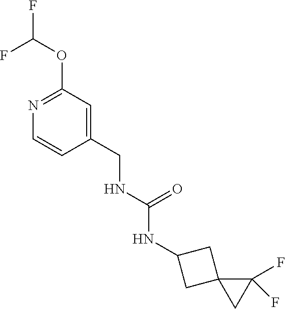 [SMILES: O=C(NCc1ccnc(OC(F)F)c1)NC1CC2(C1)CC2(F)F] |
| 16 | 1-[[2- (difluoromethoxy) pyridin-4- yl]methyl]-3-(2- fluorospiro[3.3] heptan-6-yl)urea | 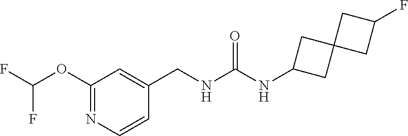 [SMILES: O=C(NCc1ccnc(OC(F)F)c1)NC1CC2(CC(F)C2)C1] |
| 17 | 1-[[2- (difluoromethoxy) pyridin-4- yl]methyl]-3-(5- oxaspiro[3.5]nonan- 8-yl)urea | 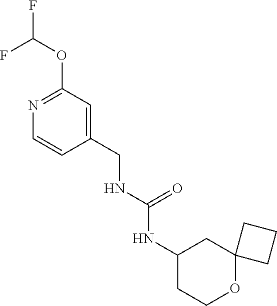 [SMILES: O=C(NCc1ccnc(OC(F)F)c1)NC1CCOC2(CCC2)C1] |
| 18 | 1-[[2- (difluoromethoxy) pyridin-4- yl]methyl]-3-[2- (hydroxymethyl) spiro[3.3]heptan-2- yl]urea | 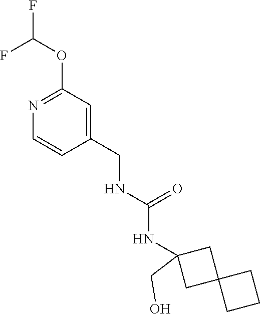 [SMILES: O=C(NCc1ccnc(OC(F)F)c1)NC1(CO)CC2(CCC2)C1] |
| 19 | 1-[2- (difluoromethoxy) pyridin-4- yl]methyl]-3-(2,2- difluorospiro[2.4] heptan-6-yl)urea | 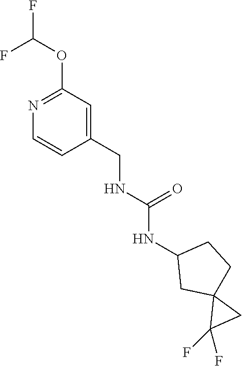 [SMILES: O=C(NCc1ccnc(OC(F)F)c1)NC1CCC2(C1)CC2(F)F] |
| 20 | 1-[[2- (difluoromethoxy) pyridin-4- yl]methyl]-3-(1- oxaspiro[3.5]nonan- 3-yl)urea | 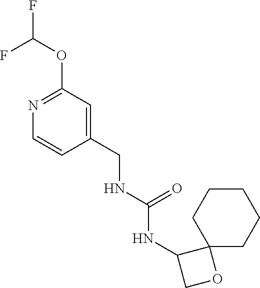 [SMILES: O=C(NCc1ccnc(OC(F)F)c1)NC1COC12CCCCC2] |
| 21 | 1-[[2- (difluoromethoxy) pyridin-4- yl]methyl]-3-(4,4- difluoro-6- oxaspiro[4.5]decan- 9-yl)urea | 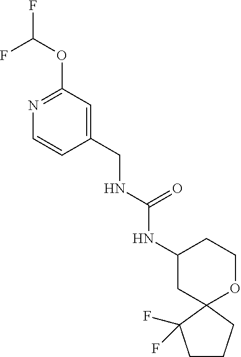 [SMILES: O=C(NCc1ccnc(OC(F)F)c1)NC1CCOC2(CCCC2(F)F)C1] |
| 22 | 1-[[2- (difluoromethoxy) pyridin-4- yl]methyl]-3-(5- oxaspiro[3.4]octan- 7-yl)urea | 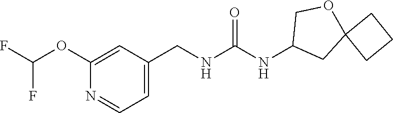 [SMILES: O=C(NCc1ccnc(OC(F)F)c1)NC1COC2(CCC2)C1] |
| 23 | 1-[[2- (difluoromethoxy) pyridin-4- yl]methyl]-3- spiro[2.3]hexan-2- ylurea | 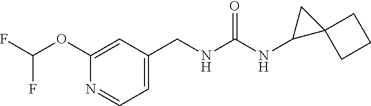 [SMILES: O=C(NCc1ccnc(OC(F)F)c1)NC1CC12CCC2] |
| 24 | 1-[[2- (difluoromethoxy) pyridin-4- yl]methyl]-3-(2,2- difluorospiro[2.3] hexan-1-yl)urea | 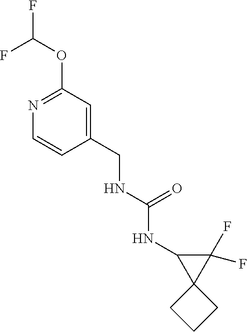 [SMILES: O=C(NCc1ccnc(OC(F)F)c1)NC1C(F)(F)C12CCC2] |
| 25 | 1-[[2- (difluoromethoxy) pyridin-4- yl]methyl]-3-[rac- (2R,3R)-3- hydroxyspiro[3.3] heptan-2-yl]urea | 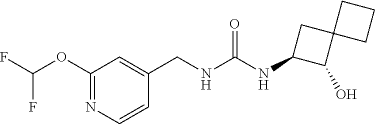 [SMILES: O=C(NCc1ccnc(OC(F)F)c1)N[C@H]1CC2(CCC2)[C@@H]1O] |
| 27 | 1-[[2- (difluoromethoxy) pyridin-4- yl]methyl]-3- [(8R)-5- oxaspiro[3.5]nonan- 8-yl]urea | 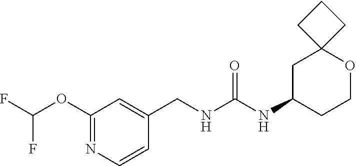 [SMILES: O=C(NCc1ccnc(OC(F)F)c1)N[C@@H]1CCOC2(CCC2)C1] |
| 28 | 1-[[2- (difluoromethoxy) pyridin-4- yl]methyl]-3- [(2R,3S)-3- hydroxyspiro[3.3] heptan-2-yl]urea | 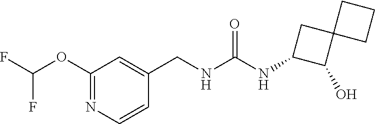 [SMILES: O=C(NCc1ccnc(OC(F)F)c1)N[C@@H]1CC2(CCC2)[C@@H]1O] |
| 29 | 1-[[2-(2,3- difluoropropoxy) pyridin-4- yl]methyl]-3- spiro[3.3]heptan- 2-ylurea | 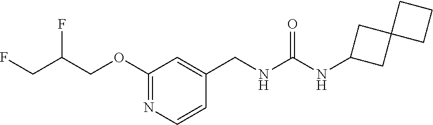 [SMILES: O=C(NCc1ccnc(OCC(F)CF)c1)NC1CC2(CCC2)C1] |
| 30 | 1-[2- (difluoromethoxy) pyridin-4- yl]methyl]-3-(1- oxaspiro[4.5]decan- 3-yl)urea | 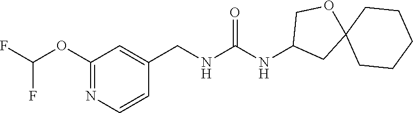 [SMILES: O=C(NCc1ccnc(OC(F)F)c1)NC1COC2(CCCCC2)C1] |
| 31 | 1-[[2- (difluoromethoxy) pyridin-4- yl]methyl]-3-(2,2- difluorospiro[2.5] octan-6-yl)urea | 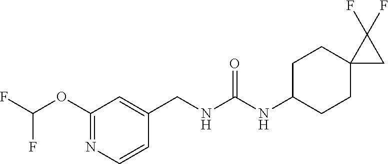 [SMILES: O=C(NCc1ccnc(OC(F)F)c1)NC1CCC2(CC1)CC2(F)F] |
| 32 | 1-[[2- (difluoromethoxy) pyridin-4- yl]methyl]-3-(5- oxaspiro[3.5]nonan- 7-yl)urea | 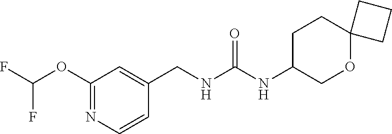 [SMILES: O=C(NCc1ccnc(OC(F)F)c1)NC1CCC2(CCC2)OC1] |

In some embodiments, the invention relates to any medical use of a compound as described herein, selected from the following list:

| | | |
|---|---|---|
| 4 | 1-[[2- (difluoromethoxy)pyridin-4- yl]methyl]-3-spiro[3.3]heptan- 2-ylurea | 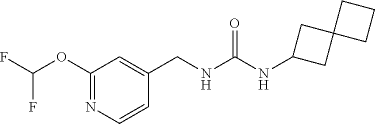 [SMILES: O=C(NCc1ccnc(OC(F)F)c1)NC1CC2(CCC2)C1] |
| 5 | 1-[[2-(2,2- difluoropropoxy)pyridin-4- yl]methyl]-3-spiro[3.3]heptan- 2-ylurea | 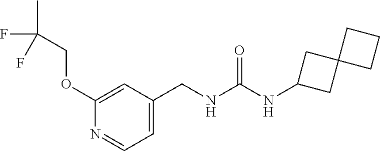 [SMILES: CC(F)(F)COc1cc(CNC(=O)NC2CC3(CCC3)C2)ccn1] |
| 6 | 1-[[2-(2- fluoropropoxy)pyridin-4- yl]methyl]-3-spiro[3.3]heptan- 2-ylurea | 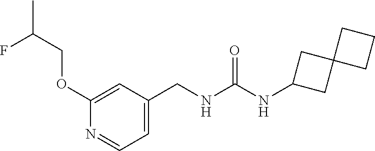 [SMILES: CC(F)COc1cc(CNC(=O)NC2CC3(CCC3)C2)ccn1] |
| 7 | 1-[[2- (cyclopropylmethoxy)pyridin-4- yl]methyl]-3-spiro[3.3]heptan- 2-ylurea | 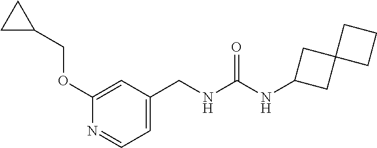 [SMILES: O=C(NCc1ccnc(OCC2CC2)c1)NC1CC2(CCC2)C1] |
| 23 | 1-[[2-(difluoromethoxy)pyridin- 4-yl]methyl]-3-spiro[2.3]hexan- 2-ylurea | 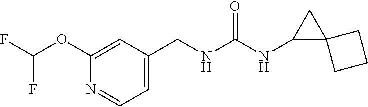 [SMILES: O=C(NCc1ccnc(OC(F)F)c1)NC1CC12CCC2] |
| 24 | 1-[[2-(difluoromethoxy)pyridin- 4-yl]methyl]-3-(2,2- difluorospiro[2.3]hexan-1- yl)urea | 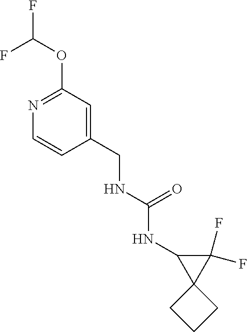 [SMILES: O=C(NCc1ccnc(OC(F)F)c1)NC1C(F)(F)C12CCC2] |
| 8 | 1-[[2- (difluoromethoxy)pyridin-4- yl]methyl]-3-spiro[2.3]hexan-5- ylurea | 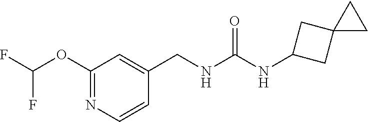 [SMILES: O=C(NCc1ccnc(OC(F)F)c1)NC1CC2(CC2)C1] |
| 9 | 1-[[2-(1,1- difluoroethyl)pyridin-4- yl]methyl]-3-spiro[3.3]heptan- 2-ylurea | 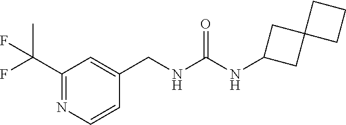 [SMILES: CC(F)(F)c1cc(CNC(=O)NC2CC3(CCC3)C2)ccn1] |
| 10 | 1-spiro[3.3]heptan-2-yl-3-[[2- (trifluoromethyl)pyridin-4- yl]methyl]urea | 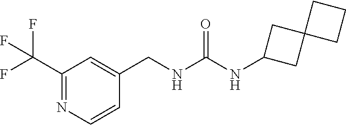 [SMILES: O=C(NCc1ccnc(C(F)(F)F)c1)NC1CC2(CCC2)C1] |
| 11 | 1-[[2-(difluoromethoxy)pyridin- 4-yl]methyl]-3-spiro[3.4]octan- 7-ylurea | 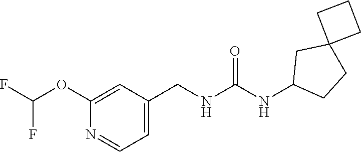 [SMILES: O=C(NCc1ccnc(OC(F)F)c1)NC1CCC2(CCC2)C1] |
| 12 | 1-spiro[2.3]hexan-5-yl-3-[[2- (2,2,2-trifluoroethoxy)pyridin- 4-yl]methyl]urea | 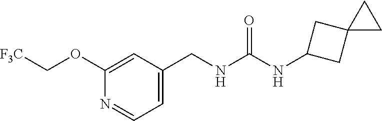 [SMILES: O=C(NCc1ccnc(OCC(F)(F)F)c1)NC1CC2(CC2)C1] |
| 13 | 1-[2-hydroxy-1-[2-(2,2,2- trifluoroethoxy)pyridin-4- yl]ethyl]-3-spiro[3.3]heptan-2- ylurea | 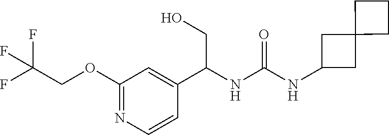 [SMILES: O=C(NC1CC2(CCC2)C1)NC(CO)c1ccnc(OCC(F)(F)F)c1] |
| 15 | 1-[[2- (difluoromethoxy)pyridin-4- yl]methyl]-3-(2,2- difluorospiro[2.3]hexan-5- yl)urea | 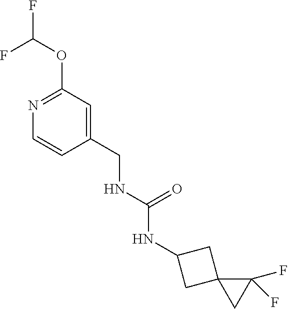 [SMILES: O=C(NCc1ccnc(OC(F)F)c1)NC1CC2(C1)CC2(F)F] |
| 16 | 1-[[2-(difluoromethoxy)pyridin- 4-yl]methyl]-3-(2- fluorospiro[3.3]heptan-6-yl)urea | 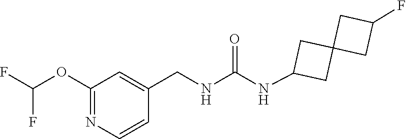 [SMILES: O=C(NCc1ccnc(OC(F)F)c1)NC1CC2(CC(F)C2)C1] |
| 18 | 1-[[2- (difluoromethoxy)pyridin-4- yl]methyl]-3-[2- (hydroxymethyl)spiro[3.3] heptan-2-yl]urea | 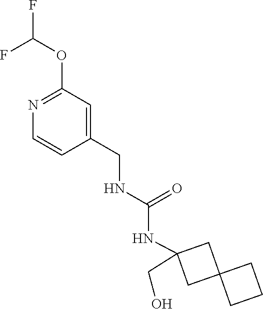 |
| 19 | 1-[[2- (difluoromethoxy)pyridin-4- yl]methyl]-3-(2,2- difluorospiro[2.4]heptan-6- yl)urea | 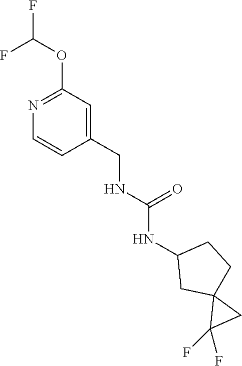 [SMILES: O=C(NCc1ccnc(OC(F)F)c1)NC1CCC2(C1)CC2(F)F] |
| 28 | 1-[[2- (difluoromethoxy)pyridin-4- yl]methyl]-3-[(2R,3S)-3- hydroxyspiro[3.3]heptan-2- yl]urea | 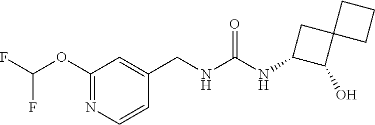 [SMILES: O=C(NCc1ccnc(OC(F)F)c1)N[C@@H]1CC2(CCC2)[C@@H]1O] |
| 29 | 1-[[2-(2,3- difluoropropoxy)pyridin-4- yl]methyl]-3-spiro[3.3]heptan- 2-ylurea | 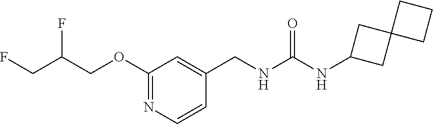 [SMILES: O=C(NCc1ccnc(OCC(F)CF)c1)NC1CC2(CCC2)C1] |

In some embodiments, the invention relates to to any medical use of a compound as described herein, selected from the following list:

| | | |
|---|---|---|
| 1 | 1-(1- oxaspiro[4.4]nonan-3- yl)-3-[(2-pyrazol-1- ylpyridin-4- yl)methyl]urea | 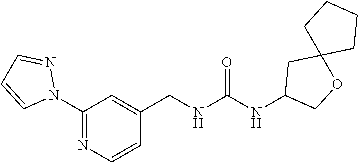 [SMILES: O=C(NCc1ccnc(-n2cccn2)c1)NC1COC2(CCCC2)C1] |
| 14 | 1-[[2- (difluoromethoxy)pyridin- 4-yl]methyl]-3-(6- oxaspiro[4.5]decan-9- yl)urea | 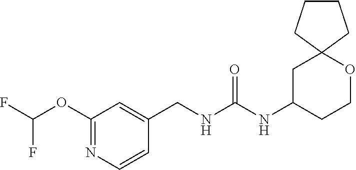 [SMILES: O=C(NCc1ccnc(OC(F)F)c1)NC1CCOC2(CCCC2)C1] |
| 17 | 1-[[2- (difluoromethoxy)pyridin- 4-yl]methyl]-3-(5- oxaspiro[3.5]nonan-8- yl)urea | 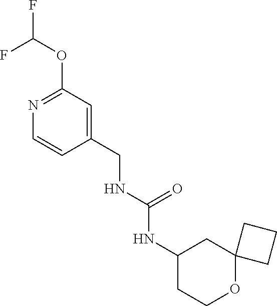 [SMILES: O=C(NCc1ccnc(OC(F)F)c1)NC1CCOC2(CCC2)C1] |
| 20 | 1-[[2- (difluoromethoxy)pyridin- 4-yl]methyl]-3-(1- oxaspiro[3.5]nonan-3- yl)urea | 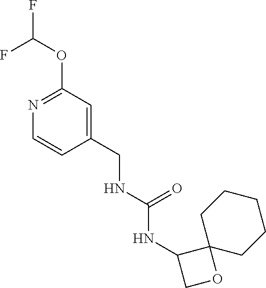 [SMILES: O=C(NCc1ccnc(OC(F)F)c1)NC1COC12CCCCC2] |
| 21 | 1-[[2- (difluoromethoxy)pyridin- 4-yl]methyl]-3-(4,4- difluoro-6- oxaspiro[4.5]decan-9- yl)urea | 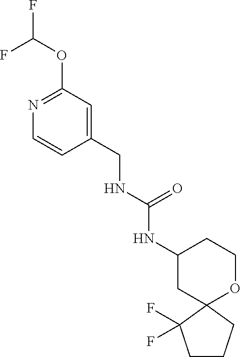 [SMILES: O=C(NCc1ccnc(OC(F)F)c1)NC1CCOC2(CCCC2(F)F)C1] |
| 22 | 1-[[2- (difluoromethoxy)pyridin- 4-yl]methyl]-3-(5- oxaspiro[3.4]octan-7- yl)urea | 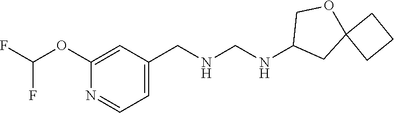 [SMILES: FC(F)Oc1cc(CNCNC2COC3(CCC3)C2)ccn1] |
| 27 | 1-[[2- (difluoromethoxy)pyridin- 4-yl]methyl]-3-[(8R)- 5-oxaspiro[3.5]nonan-8- yl]urea | 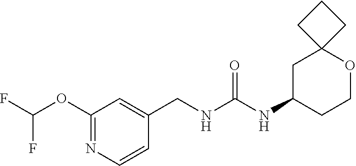 [SMILES: O=C(NCc1ccnc(OC(F)F)c1)N[C@@H]1CCOC2(CCC2)C1] |
| 30 | 1-[[2- (difluoromethoxy)pyridin- 4-yl]methyl]-3-(1- oxaspiro[4.5]decan-3- yl)urea | 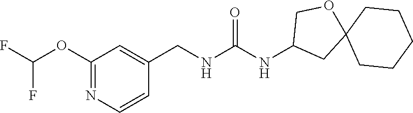 [SMILES: O=C(NCc1ccnc(OC(F)F)c1)NC1COC2(CCCCC2)C1] |
| 32 | 1-[[2- (difluoromethoxy)pyridin- 4-yl]methyl]-3-(5- oxaspiro[3.5]nonan-7- yl)urea | 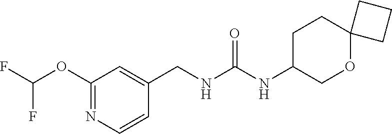 [SMILES: O=C(NCc1ccnc(OC(F)F)c1)NC1CCC2(CCC2)OC1] |

In some embodiments the compound of formula (1) is not:

| | | |
|---|---|---|
| 12 | 1-spiro[2.3]hexan-5-yl-3-[[2- (2,2,2-trifluoroethoxy)pyridin- 4-yl]methyl]urea | 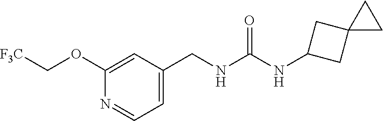 [SMILES: O=C(NCc1ccnc(OCC(F)(F)F)c1)NC1CC2(CC2)C1] |

In some embodiments, the invention relates to a compound as described herein, wherein such compounds of formula (I) show Kv7.2 EC₅₀: <1 μM or <3 μM, as most preferred or preferred EC₅₀. In some embodiments such compounds show Kv7.2 EC₅₀: <3 μM.

### Combination Therapy

Compounds of the invention may be combined with one or more other compounds of the invention or one or more other therapeutic agent as any combination thereof, in the treatment of the diseases, disorders, or disabilities provided herein. For example, a compound of the invention may be administered simultaneously, sequentially or separately in combination with other therapeutic agents known to be useful for the treatment of a disease or disorder selected from those recited herein.

In some embodiments, compounds of the invention may be combined with another therapeutically active agent having a synergistic effect in the treatment of any diseases, disorders, or disabilities described herein.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Articles of Manufacture

In one embodiment, the present invention provides an article of manufacture, or “kit”, containing materials useful for the treatment of the disorder, disease, or disability described herein is provided.

In one embodiment of the invention, the kit comprises a container comprising a compound of this invention, as described in any embodiment of this invention.

In one embodiment, the present invention provides a kit comprising a container comprising a compound of this invention, or a pharmaceutical composition thereof, as described herein.

In one embodiment, the present invention provides a kit comprising a compound of this invention, any exemplified compound, any embodiment, or combinations of embodiments, or pharmaceutical composition thereof, as described herein.

In one embodiment, the present invention provides a kit, wherein the compound is selected from formulae (I)-(VII), or a solvate, a pharmaceutically acceptable salt, or a pharmaceutical composition thereof, as described herein.

In one embodiment, the present invention provides a kit, wherein the compound is selected from Table 1, 2, and 3, a solvate, a pharmaceutically acceptable salt, or a pharmaceutical composition thereof, as described herein.

In one embodiment, the present invention provides a kit for use in the treatment of a disorder, disease, or disability associated with Kv7.2, comprising:
a) a first pharmaceutical composition comprising a compound of this invention; and
b) instructions for use.

In one embodiment, the present invention provides a kit for use in the therapeutic and/or prophylactic treatment of a disorder, disease, or disability associated with Kv7.2 comprising:
b) a compound of this invention, or a pharmaceutical composition, or a pharmaceutical composition for use thereof, as described herein; and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising:
a) a first pharmaceutical composition comprising a compound of formula (I), or a solvate or a pharmaceutically acceptable salt, a pharmaceutical composition, or a pharmaceutical composition for use; as described herein:

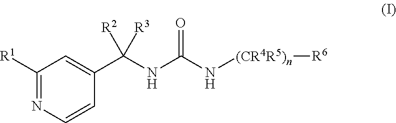

wherein
R¹ is selected from a 5-6 membered heteroaryl, haloC₁₋₆alkoxy, C₃₋₅cycyloxy, cyano, haloC₁₋₆alkyl, halogen, C₁₋₆alkyl, C₁₋₆alkoxy, and C₃₋₅heterocyclyloxy, wherein such heteroaryl, and cyclyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl and hydroxy;
and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising: b) a first pharmaceutical composition comprising a compound of formula (I), or a solvate or a pharmaceutically acceptable salt, a pharmaceutical composition, or a pharmaceutical composition for use; as described herein:

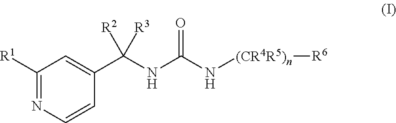

wherein
R¹ is selected from a 5-6 membered heteroaryl, haloC₁₋₆alkoxy, C₃₋₅cycloalkylC₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocylyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl and hydroxy;
and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising: c) a first pharmaceutical composition comprising a compound of formula (I), or a solvate or a pharmaceutically acceptable salt, a pharmaceutical composition, or a pharmaceutical composition for use; as described herein:

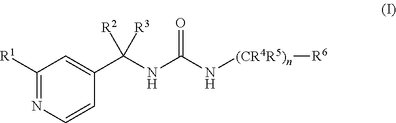

wherein
R¹ is selected from a 5-6 membered heteroaryl, haloC₁₋₆alkoxy, cyano, haloC₁₋₆alkyl, halogen, C₁₋₆alkyl, and C₁₋₆alkoxy, wherein such heteroaryl is optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆alkyl and hydroxy;
and
b) instructions for use.

In preferred embodiments, n is 0.

In one embodiment, the present invention provides a kit for use in the therapeutic and/or prophylactic treatment of a disorder, disease, or disability associated with Kv7.2. These disorders, diseases or disabilities can be selected from behavioral disorders, mood disorders, neurodevelopmental disorders, intellectual disability, epilepsies, neurodegenerative diseases, pain, migraine, and tinnitus, comprising a compound, a pharmaceutical composition, or a pharmaceutical composition for use thereof, as described herein.

In one embodiment, the present invention provides a kit for use as described herein, wherein the behavioral disorder Attention Deficit Hyperactivity Disorder (ADHD).

In one embodiment, the present invention provides a kit for use as described herein, wherein the mood disorder is depression.

In one embodiment, the present invention provides a kit for use as described herein, wherein the neurodevelopment disorder is selected from autism spectrum disorder (ASD) and syndromic developmental disorders.

In one embodiment, the present invention provides a kit for use as described herein, wherein the syndromic developmental disorder is selected from Dup15q syndrome (Dup15q), Fragile X syndrome (FXS), and Angelman syndrome.

In one embodiment, the present invention provides a kit for use as described herein, wherein the epilepsies are selected from broad pediatric epilepsy, West syndrome, Ohtahara syndrome, and epileptic encephalopathy.

In one embodiment, the present invention provides a kit for use as described herein, wherein the neurodegenerative diseases are selected from Alzheimer's disease, and motor neuron diseases.

In one embodiment, the present invention provides a kit for use as described herein, wherein the kit further comprises a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products.

Suitable containers include, e.g., bottles, vials, syringes, blister pack, etc. The container may be formed from a variety of materials such as glass or plastic.

The container may hold a compound of this invention or a formulation thereof which is effective for treating the condition and may have a sterile access port (e.g., the container may be an intravenous solution bag or a vial having a stopper pierceable by a hypodermic injection needle). At least one active agent in the composition is a compound of this invention. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of this invention can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit further comprises directions for the administration of the compounds of this invention and, if present, the second pharmaceutical formulation. For example, if the kit comprises a first composition comprising a compound of this invention, and a second pharmaceutical formulation, the kit may further comprise directions for the simultaneous, sequential or separate administration of the first and second pharmaceutical compositions to a patient in need thereof.

In one embodiment, the present invention provides a kit for use as described herein, wherein the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

In one embodiment, the present invention provides a kit for use as described herein, wherein the kit comprises (a) a first container with a compound of this invention contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit comprises a composition of this invention and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Methods of Manufacturing

In a further aspect, the present invention provides a compound of formula (I), or a solvate or a pharmaceutically acceptable salt, when manufactured according to a process described herein.

The preparation of compounds of formula (I), or solvates or pharmaceutically acceptable salts thereof, may be carried out in sequential or convergent synthetic routes.

Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

In any of the embodiments relating to schemes or examples described in this application relating to a compound of this invention, any embodiment can be combined with any other embodiment unless contradictory.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC, or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of this invention, i.e. compounds selected from formulae (I)-(VII), or their solvates or pharmaceutically acceptable salts, can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis is not described in literature, they can be prepared in analogy to existing procedures for close analogues or as outlined in the experimental section.

### Abbreviations

EtOAc is ethyl acetate
CDI is 1,1′-carbonyldiimidazole
DCM is dichloromethane
DIPEA is N,N-diisopropylethylamine
DMF is N,N-dimethylformamide
HCl is hydrogen chloride
HPLC is high pressure liquid chromatography
LCMS is liquid chromatography mass spectrometry
NaHCO₃ is sodium hydrogen carbonate
NaOH is sodium hydroxide
MeOH is methanol
MgSO₄ is magnesium sulfate
PYBROP is bromotripyrrolidinophosphonium hexafluorophosphate
o/n is overnight
RT is room temperature
TLC is thin-layer chromatography
CHO is chinese hamster ovary
CMV is cytomegalovirus
FBS is fetal bovine serum
NEAA is non-essential amino acids
NaCl is sodium chloride
KCl is potassium chloride
CaCl₂ is calcium chloride
MgCl₂ is magnesium chloride
HEPES is 4-(2-hydroxyethyl)-1-piperazine ethanesulfonic acid
NMDG is N-methyl-D-glucamine diatrizoate
EGTA is ethylene glycol-bis(Q-aminoethyl ether)-N,N,N′,N′-tetraacetic acid
EDTA is ethylenediaminetetraacetic acid
DPBS is Dulbecco's phosphate-buffered saline
mV is millivolt
TEA is tetraethylammonium
NADPH is nicotinamide adenine dinucleotide phosphate
CLᵢₙₜ is intrinsic clearance

The present invention provides compounds of formula (I), or a solvate or a pharmaceutically acceptable salts thereof:

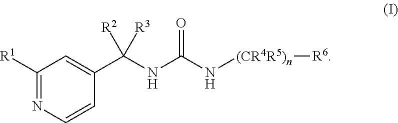

The preparation of compounds of formula (I), or solvates or pharmaceutically acceptable salts thereof may be carried out by reacting an amine with an isocyanate (Scheme 1), or by reacting one amine with carbonyl di-imidazole followed by addition of the second amine in-situ, or by first reacting an amine with para-nitro-phenyl chloroformate or phenyl chloroformate to yield the corresponding carbamate intermediate, which can be purified or used in situ by addition of a second amine. Syntheses of the compounds of the invention are shown in the following Schemes and in the description of 32 specific examples. The skills required for carrying out the reaction and purification of the resulting products are known to those skilled in the art.

In more detail, the compounds this invention, in particular compounds selected from formulae (I)-(VII), or solvates or pharmaceutically acceptable salts thereof, can be manufactured by the methods given in the examples or by analogous methods. Starting materials are either commercially available or can be prepared by methods analogous to the methods given below or by methods known in the art.

### General Procedures

For more information on the general procedures, please refer to the embodiments relating to the process of making compounds of formula (I), or solvates or pharmaceutically acceptable salts thereof as described herein.

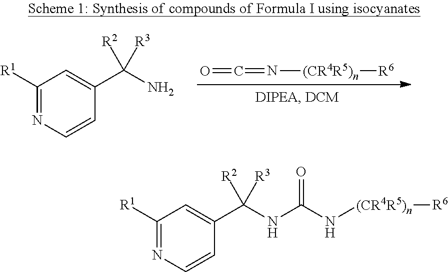

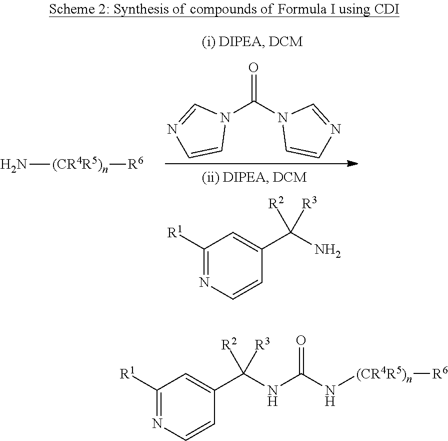

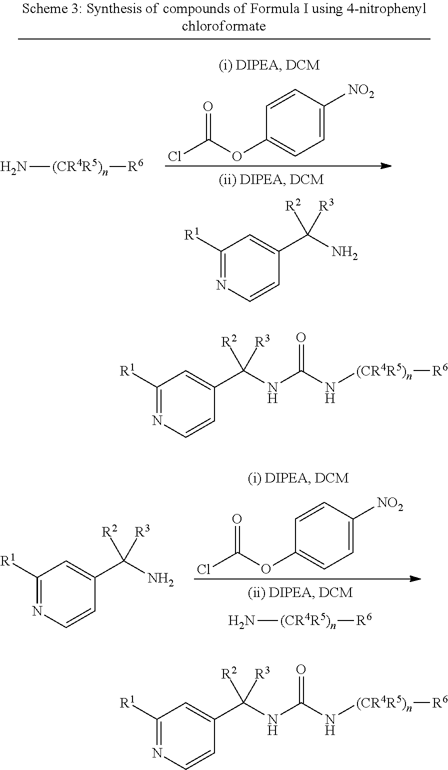

wherein R¹, R², R³, R⁴, R⁵, n, and R⁶ are as defined herein.

These reactions are preferably used for R¹ being selected from 7-12 membered heterocycloalkyl, cyano, halogen, haloC₁₋₆alkyl, haloC₁₋₆alkoxy, and 5 membered heteroaryl.

The urea formation can be accomplished by treatment of an amine (or the corresponding salt, such as HCl) with an isocyanate (Scheme 1) in DCM or DMF at temperatures from RT to 40° C., or by reacting the first amine with 1,1′-carbonyldiimidazole (Scheme 2) in a solvent (DCM, AcN, THF) and in the presence of a suitable base (DIPEA, NEt3) to generate the activated urea prior to the addition of the second amine (or the corresponding salt), or by reacting the first amine with para-nitrophenyl chloroformate (Scheme 3) or phenyl chloroformate in a solvent (AcN, THF) and in the presence of a base (DIPEA, NEt3), to generate the carbamate which can be purified or used in situ with a second amine to yield the desired urea.

Preferred conditions are using CDI with DIPEA as a base and with DCM as a solvent at 0° C. for 45 min and then adding the second amine and stirring at 40° C. for 2-6 hours.

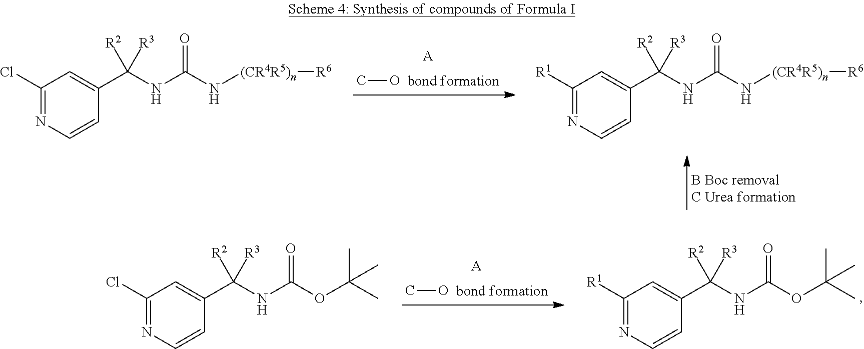

wherein this reaction is used for the preparation of compounds with R¹ being any alkoxy group described herein, e.g. haloC₁₋₆alkoxy, C₁₋₆alkoxy, 4-6 membered heterocyclyloxy, halogen, or C₃₋₆cycloalkylC₁₋₆alkoxy. In some embodiments, this reaction is applied in cases when R² and R³ are H.

In some embodiments, R² and R³ are as described herein other than H.

Step A: The C—O bond formation can be accomplished via Buchwald-Hartwig etherification between 2-chloro-pyridyl intermediate and a primary alcohol using a Palladium catalyst and an inorganic base in toluene at 80° C.

Preferred conditions are using tBuBrettPhosPd G3 as a catalyst and cesium carbonate.

Step B: Boc deprotection can be carried out by treatment with HCl/Dioxane.

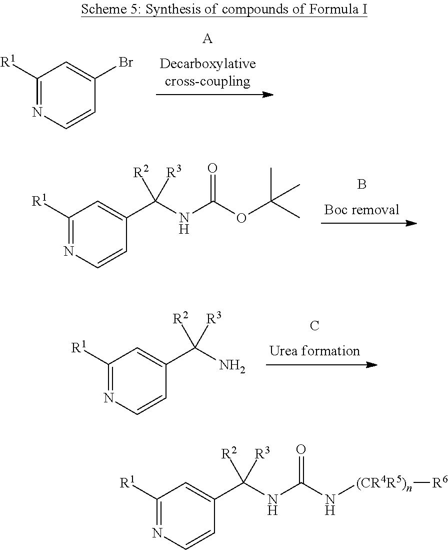

wherein R¹, R², R³, R⁴, R⁵, n, and R⁶ are as defined herein which this reaction is used for the preparation of compounds with R¹ being any alkoxy group described herein, e.g. haloC₁₋₆alkoxy, C₁₋₆alkoxy, 4-6 membered heterocyclyloxy, halogen, C₃₋₅cycloalkylC₁₋₆alkoxy, or cyanoC₁₋₆alkoxy.

In one preferred embodiment is used when R² is hydroxyC₁₋₆alkyl and R³ is hydrogen.

Step A: The decarboxylative cross-coupling reaction can be performed between an heteroaryl halide and protected alpha-amino acids under the combined action of visible-light photoredox-Ni catalysis and in the presence of cesium carbonate and an Iridium photocatalyst.

Step B: Boc deprotection can be carried out by treatment with HCl/Dioxane.

Step C: Urea formation can be performed out via schemes 1-3. Preferred conditions are using CDI with DIPEA as a base and with DCM as a solvent at 0° C. for 1 hour and then adding the second amine and stirring at RT overnight. In cases where R² or R³ is protected as a benzyl ether, deprotection to the alcohol can be performed by hydrogenation in the presence of 10% Palladium on charcoal in Ethanol/Ethyl acetate, or with BBr₃ in DCM.

### Isolation and Purification of the Compounds

Isolation and purification of the compounds and intermediates described herein can be carried out, if desired, by any suitable separation or purification procedure such as, for example, filtration, extraction, crystallization, column chromatography, thick-layer chromatography, preparative low or high-pressure liquid chromatography or a combination of these procedures. However, other equivalent separation or isolation procedures could, of course, also be used. Mixture of chiral compounds of formula (I) can be separated using preparative chiral HPLC purifications. Chiral HPLC purifications were performed on an AccQPrep HP125 (Teledyne ISCO) system, with a 5 μm 250 mm×21.2 mm i.d. chiral column (Amylose-1, Cellulose-1, or Cellulose-4) from Phenomenex, running at a flow rate of 20.8 mL min-1 with UV (214 and 254 nm) and ELS detection. Eluents: water; acetonitrile.

### EXAMPLES

### Example 1: 1-(1-oxaspiro[4.4]nonan-3-yl)-3-[(2-pyrazol-1-ylpyridin-4-yl)methyl]urea

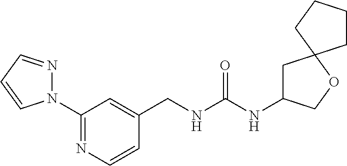 [SMILES: O=C(NCc1ccnc(-n2cccn2)c1)NC1COC2(CCCC2)C1]

To (2-(1H-pyrazol-1-yl)pyridin-4-yl)methanamine (52 mg, 0.3 mmol) in DCM (0.2 ml) in an ice bath was added phenyl chloroformate (47 mg, 0.3 mmol) followed by DIPEA (0.1 ml, 0.6 mmol). The reaction was stirred at −0° C. for 45 min. 1-oxaspiro[4.4]nonan-3-amine (42 mg, 0.3 mmol) was then added and the reaction was stirred at 40° C. for 3 hours. The solvent was removed in vacuo. DMF (2 ml) was added and purification by preparative HPLC afforded the title compound as a white solid (42% yield).

MS (m/z): 341.3 [M+H]+.

### Example 2: 1-(6-oxaspiro[4.5]decan-9-yl)-3-[(2-pyrazol-1-ylpyridin-4-yl)methyl]urea

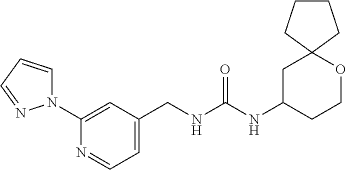 [SMILES: O=C(NCc1ccnc(-n2cccn2)c1)NC1CCOC2(CCCC2)C1]

[2-(pyrazol-1-yl)pyridin4-yl]methanamine (75 mg, 0.43 mmol, 1 eq) was dissolved in dry DCM (3 mL) with DIPEA (0.37 mL, 2.15 mmoles) then cooled to 0° C. under N₂. The reactions was then treated with 1,1′-carbonyldiimidazole (69.81 mg, 0.43 mmol, 1 eq) and stirred at 0° C. for 45 mins. The reaction was then treated with 6-oxaspiro[4.5]decan-9-amine (66.8 mg, 0.43 mmol, 1 eq) then warmed to 35° C. for 16 h. The reaction was concentrated in vacuo, dissolved in 2 ml DMSO and purified by preparative HPLC to yield the titled compound (53.9% yield) as a white solid.

MS (m/z): 356.2 [M+H]+.

### Example 3: 1-[[2-(difluoromethoxy)pyridin-4-yl]methyl]-3-(1-oxaspiro[4.4]nonan-3-yl)urea

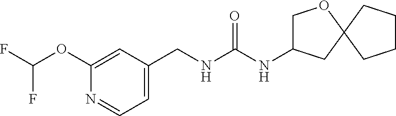 [SMILES: O=C(NCc1ccnc(OC(F)F)c1)NC1COC2(CCCC2)C1]

The title compound was obtained in analogy to Example 2 as a white solid (55% yield) using (2-(difluoromethoxy)pyridin-4-yl)methanamine, 1-oxaspiro[4.4]nonan-3-amine and 1,1′-carbonyldiimidazole.

MS (m/z): 341.9 [M+H]+.

### Example 4: 1-[[2-(difluoromethoxy)pyridin-4-yl]methyl]-3-spiro[3.3]heptan-2-ylurea

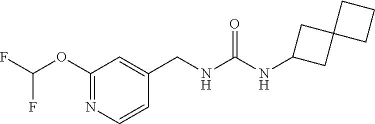 [SMILES: O=C(NCc1ccnc(OC(F)F)c1)NC1CC2(CCC2)C1]

The title compound was obtained in analogy to Example 2 as a white solid (49% yield) using (2-(difluoromethoxy)pyridin-4-yl)methanamine, spiro[3.3]heptan-2-amine hydrochloride and 1,1′-carbonyldiimidazole.

MS (m/z): 312.2 [M+H]+.

### Example 5: 1-[[2-(2,2-difluoropropoxy)pyridin-4-yl]methyl]-3-spiro[3.3]heptan-2-ylurea

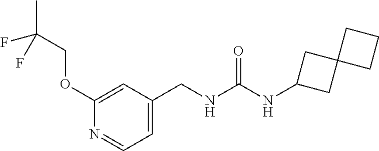 [SMILES: CC(F)(F)COc1cc(CNC(=O)NC2CC3(CCC3)C2)ccn1]

### a) 1-((2-chloropyridin-4-yl)methyl)-3-(spiro[3.3]heptan-2-yl)urea

The title compound was obtained in analogy to Example 2 as a white solid (83% yield) using (2-chloropyridin-4-yl)methanamine hydrochloride, spiro[3.3]heptan-2-amine hydrochloride and 1,1′-carbonyldiimidazole.

MS (m/z): 280 [M+H]+.

### b) 1-[[2-(2,2-difluoropropoxy)pyridin-4-yl]methyl]-3-spiro[3.3]heptan-2-ylurea 1-((2-chloropyridin-4-yl)methyl)-3-(spiro[3.3]heptan-2-yl)urea (70 mg, 0.25 mmol), tBuBrettPhosPd G3 (6.41 mg, 0.01 mmol) and cesium carbonate (122.3 mg, 0.38 mmol) were combined in a dry 10 ml flask. This was flushed with nitrogen before adding Toluene (1.5 mL) and 2,2-difluoropropan-1-ol (30.05 μL, 1.2 g/mL, 0.38 mmol). The resultant brown mixture was heated at 80° C. under N₂. The reaction was complete after 30 min. The reaction mixture was allowed to cool then diluted with CH₂Cl₂ and filtered through Celite. The solvent was removed in vacuo to afford a crude solid (90 mg), which was purified by flash chromatography with silica eluting with heptane-50% EtOAc/heptane (gradient) to afford the desired product as a white solid (30 mg, 35% yield)

MS (m/z): 340.2 [M+H]+.

### Example 6: 1-[[2-(2-fluoropropoxy)pyridin-4-yl]methyl]-3-spiro[3.3]heptan-2-ylurea

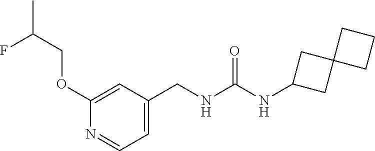 [SMILES: CC(F)COc1cc(CNC(=O)NC2CC3(CCC3)C2)ccn1]

The title compound was obtained in analogy to Example 5 as a while solid (18% yield) using (1-[[2-chloropyridin-4-yl]methyl]-3-spiro[3.3]heptan-2-ylurea and 2-fluoropropan-1-ol.

MS (m/z): 322.2 [M+H]+.

### Example 7: 1-[[2-(cyclopropylmethoxy)pyridin-4-yl]methyl]-3-spiro[3.3]heptan-2-ylurea

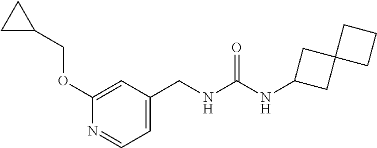 [SMILES: O=C(NCc1ccnc(OCC2CC2)c1)NC1CC2(CCC2)C1]

The title compound was obtained in analogy to Example 5 as a while solid (33% yield) using (1-[[2-chloropyridin-4-yl]methyl]-3-spiro[3.3]heptan-2-ylurea and cyclopropylmethanol.

MS (m/z): 316.2 [M+H]+.

### Example 8: 1-[[2-(difluoromethoxy)pyridin-4-yl]methyl]-3-spiro[2.3]hexan-5-ylurea

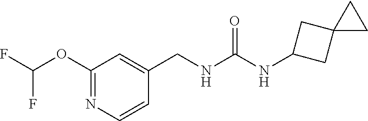 [SMILES: O=C(NCc1ccnc(OC(F)F)c1)NC1CC2(CC2)C1]

### a) 4-nitrophenyl N-{[2-(difluoromethoxy)pyridin4-yl]methyl}carbamate

A solution of [2-(difluoromethoxy)pyridin-4-yl]methanamine (3 g, 17.23 mmol, 1 eq) and NEt₃ (4.79 mL, 34.45 mmol, 2 eq) in dry DCM (20 mL) was slowly added to a solution of 4-Nitrophenyl chloroformate (3.47 g, 17.23 mmol, 1 eq) in dry DCM (20 mL) at 0° C. The reaction was left to stir at 0° C. for 1 h then left to warm to RT for 48 hours. The reaction was partitioned between sat NaHCO₃ (aq) and DCM, extracted with DCM (3×150 mL) and organic layers were combined, washed with brine, dried and concentrated in vacuo to afford a yellow gum. Crude material was purified by automated flash chromatography on Silica eluting with 0 to 60% Ethyl Acetate in Heptane to afford 4-nitrophenyl N-{[2-(difluoromethoxy)pyridin-4-yl]methyl}carbamate (2.34 g, 6.9 mmol, 40.0% yield) as a pale yellow solid.

MS (m/z): 340.2 [M+H]+.

### b) 1-[[2-(difluoromethoxy)pyridin-4-yl]methyl]-3-spiro[2.3]hexan-5-ylurea

4-nitrophenyl N-{[2-(difluoromethoxy)pyridin-4-yl]methyl}carbamate (70 mg, 0.21 mmol, 1 eq) was added to a solution of spiro[2.3]hexan-5-amine hydrochloride (27.6 mg, 0.21 mmol, 1 eq) and NEt₃ (86.12 μL, 0.62 mmol, 3 eq) in DCM (3 mL). The reaction mixture was stirred at RT overnight. The reaction was then diluted with 50 mL water and extracted with DCM (3×30 mL). The combined organics were washed with brine, dried and concentrated in vacuo. Crude material was purified by automated flash chromatography on eluting with 0 to 100% Ethyl Acetate in Heptane to afford the title product (28.7 mg, 0.1 mmol, 46.7%) as a white solid.

MS (m/z): 298.1 [M+H]+.

### Example 9: 1-[[2-(1,1-difluoroethyl)pyridin-4-yl]methyl]-3-spiro[3.3]heptan-2-ylurea

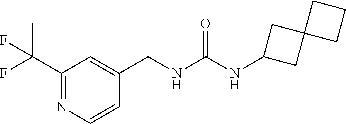 [SMILES: CC(F)(F)c1cc(CNC(=O)NC2CC3(CCC3)C2)ccn1]

1-[2-(1,1-difluoroethyl)pyridin-4-yl]methanamine (43.04 mg, 0.25 mmol, 1 eq) was dissolved in dry DCM (1.0 mL) and a solution of 2-isocyanatospiro[3.3]heptane (34.29 mg, 0.25 mmol, 1 eq) in dry DCM (1.0 mL) was slowly added and stirred at rt for 2 h. The reaction was concentrated, and the crude was purified by preparative HPLC to yield the title product (30.0 mg, 38.8%) as a white solid.

MS (m/z): 310.2 [M+H]+.

### Example 10: 1-spiro[3.3]heptan-2-yl-3-[[2-(trifluoromethyl)pyridin-4-yl]methyl]urea

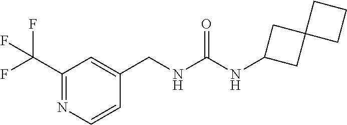 [SMILES: O=C(NCc1ccnc(C(F)(F)F)c1)NC1CC2(CCC2)C1]

The title compound was obtained in analogy to Example 9 as a while solid (41% yield) using (2-(trifluoromethyl)pyridin-4-yl)methanamine and 2-isocyanatospiro[3.3]heptane.

MS (m/z): 314.2 [M+H]+.

### Example 11: 1-[[2-(difluoromethoxy)pyridin-4-yl]methyl]-3-spiro[3.4]octan-7-ylurea

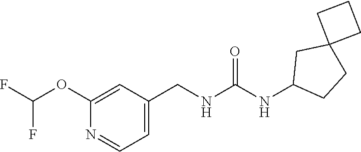 [SMILES: O=C(NCc1ccnc(OC(F)F)c1)NC1CCC2(CCC2)C1]

The title compound was obtained in analogy to Example 2 as a while solid (59% yield) using (2-(difluoromethoxy)pyridin-4-yl)methanamine, spiro[3.4]octan-6-amine and 1,1′-carbonyldiimidazole.

MS (m/z): 326.2 [M+H]+.

### Example 12: 1-spiro[2.3]hexan-5-yl-3-[[2-(2,2,2-trifluoroethoxy)pyridin-4-yl]methyl]urea

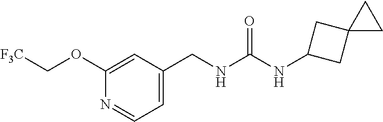 [SMILES: O=C(NCc1ccnc(OCC(F)(F)F)c1)NC1CC2(CC2)C1]

The title compound was obtained in analogy to Example 2 as a while solid solid (56% yield) using (2-(2,2,2-trifluoroethoxy)pyridin-4-yl)methanamine hydrochloride, spiro[2.3]hexan-5-amine hydrochloride and 1,1′-carbonyldiimidazole.

MS (m/z): 330.1 [M+H]+.

### Example 13: 1-[2-hydroxy-1-[2-(2,2,2-trifluoroethoxy)pyridin-4-yl]ethyl]-3-spiro[3.3]heptan-2-ylurea

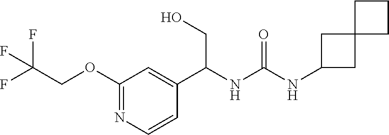 [SMILES: O=C(NC1CC2(CCC2)C1)NC(CO)c1ccnc(OCC(F)(F)F)c1]

### a) tert-butyl N-[2-(benzyloxy)-1-[2-(2,2,2-trifluoroethoxy)pyridin-4-yl]ethyl]carbamate

To a stirred solution of 4-bromo-2-(2,2,2-trifluoroethoxy)pyridine (512 mg, 2 mmol) in DMF (20 mL) was added N-Boc-O-benzyl-L-serine 97% (886 mg, 3 mmol), Nickel (II) Chloride dimethoxyethane adduct (22 mg, 0.1 mmol), cesium carbonate (977.5 mg, 3 mmol), (4,4′-Di-t-butyl-2,2′-bipyridine)bis[3,5-difluoro-2-[5-trifluoromethyl-2-pyridinyl-kN)phenyl-C]iridium(III) hexafluorophosphate (22.4 mg, 0.02 mmol), and 4,4′-di-tert-butyl-2,2′-dipyridyl (26.8 mg, 0.1 mmol). The mixture was sparged with nitrogen for 15 mins, then the septum cap was sealed with parafilm and the reaction was subjected to blue light (30 W, 450 nm) irradiation with liquid cooling at 25° C. After hours irradiation was stopped, and the mixture was poured onto saturated aqueous NaHCO₃ (200 mL) and extracted with ether (3×75 mL). The combined organics were successively washed with water and brine, dried (MgSO₄) and evaporated to afford a yellow oil that was redissolved in DCM, evaporated onto silica and purified by flash chromatography eluting with 0 to 74% Ethyl Acetate in Heptane to yield tert-butyl N-[2-(benzyloxy)-1-[2-(2,2,2-trifluoroethoxy)pyridin-4-yl]ethyl]carbamate (451 mg, 57.2%) as a yellow oil.

MS (m/z): 427 [M+H]+.

### b) 2-(benzyloxy)-1-(2-(2,2,2-trifluoroethoxy)pyridin-4-yl)ethan-1-amine

To a stirred solution of tert-butyl N-[2-(benzyloxy)-1-[2-(2,2,2-trifluoroethoxy)pyridin-4-yl]ethyl]carbamate (454 mg, 1.06 mmol) in Ethyl Acetate (10 mL) was added HCl (4M in Dioxane) (5.32 mL (4M), 21.3 mmol) and the reaction was allowed to stir overnight. The reaction mixture was diluted with ether (10 mL), filtered, washed with ether (×3) and sucked dry. The resultant solid was dried at 40° C. under high vacuum overnight to afford 2-(benzyloxy)-1-(2-(2,2,2-trifluoroethoxy)pyridin-4-yl)ethan-1-amine (379 mg, 90%) as a white solid.

MS (m/z): 327 [M+H]+.

### c) 1-2-(benzyloxy)-1-[2-(2,2,2-trifluoroethoxy)pyridin4-yl]ethyl]-3-{spiro[3.3]heptan-2-yl}urea

To a solution of 2-(benzyloxy)-1-(2-(2,2,2-trifluoroethoxy)pyridin-4-yl)ethan-1-amine (81.58 mg, 0.25 mmol, 1 eq) and DIPEA (0.09 mL, 0.5 mmol, 2 eq) in DCM (2 mL) was slowly added a solution of 2-isocyanatospiro[3.3]heptane (34.29 mg, 0.25 mmol, 1 eq) in dry DCM (1.0 mL). The reaction mixture was stirred at RT for 3 hours. The reaction was then concentrated and the crude was purified by preparative HPLC eluting with 5 to 95% ACN/0.1% FA in Water/0.1% FA to yield 1-[2-(benzyloxy)-1-[2-(2,2,2-trifluoroethoxy)pyridin-4-yl]ethyl]-3-{spiro[3.3]heptan-2-yl}urea (70 mg, 0.15 mmol, 60.4%).

MS (m/z): 464.4 [M+H]+.

### d) 1-[2-hydroxy-1-[2-(2,2,2-trifluoroethoxy)pyridin-4-yl]ethyl]-3-spiro[3.3]heptan-2-ylurea

1-[2-(benzyloxy)-1-[2-(2,2,2-trifluoroethoxy)pyridin4-yl]ethyl]-3-{spiro[3.3]heptan-2-yl}urea (60 mg, 0.13 mmol, 1 eq) was dissolved in a mixture of ethanol (1 mL) and Ethyl Acetate (1 mL). Palladium (13.78 mg (10%), 0.01 mmol, 0.1 eq) was added, the reaction vessel was sealed and hydrogen (31.51 mL, 1.29 mmol, 10 eq) added. The reaction was left to stir at rt overnight. The next 15 day the reaction was heated to 50 C and repurged with hydrogen (31.51 mL, 1.29 mmol, 10 eq) for 1.5 h. After 1.5 h, the reaction was filtered through celite and washed with EtOAc, the filtrate was concentrated and the crude was purified by preparative HPLC eluting with 5 to 95% ACN/0.1% FA in Water/0.1% FA to afford the title product (20 mg, 0.05 mmol, 41.4%) as a white solid.

MS (m/z): 374.2 [M+H]+.

### Example 14: 1-[[2-(difluoromethoxy)pyridin-4-yl]methyl]-3-(6-oxaspiro[4.5]decan-9-yl)urea

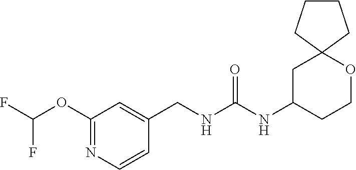 [SMILES: O=C(NCc1ccnc(OC(F)F)c1)NC1CCOC2(CCCC2)C1]

The title compound was obtained in analogy to Example 2 as a white solid (53% yield) solid using (2-(difluoromethoxy)pyridin-4-yl)methanamine, 6-oxaspiro[4.5]decan-9-amine and 1,1′-carbonyldiimidazole.

MS (m/z): 356.2 [M+H]+.

### Example 15: 1-[[2-(difluoromethoxy)pyridin-4-yl]methyl]-3-(2,2-difluorospiro[2.3]hexan-5-yl)urea

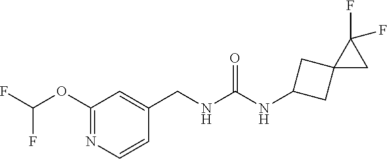 [SMILES: O=C(NCc1ccnc(OC(F)F)c1)NC1CC2(C1)CC2(F)F]

The title compound was obtained in analogy to Example 2 as a while solid (67% yield) using (2-(difluoromethoxy)pyridin-4-yl)methanamine, 1,1-difluorospiro[2.3]hexan-5-amine hydrochloride and 1,1′-carbonyldiimidazole.

MS (m/z): 334.1 [M+H]+.

### Example 16: 1-[[2-(difluoromethoxy)pyridin-4-yl]methyl]-3-(2-fluorospiro[3.3]heptan-6-yl)urea

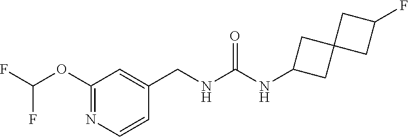 [SMILES: O=C(NCc1ccnc(OC(F)F)c1)NC1CC2(CC(F)C2)C1]

The title compound was obtained in analogy to Example 8 as a white solid (86% yield) using (2-(difluoromethoxy)pyridin-4-yl)methanamine, 6-fluorospiro[3.3]heptan-2-amine hydrochloride and para-nitrophenyl chloroformate.

MS (m/z): 330.2 [M+H]+.

### Example 17: 1-[[2-(difluoromethoxy)pyridin-4-yl]methyl]-3-(5-oxaspiro[3.5]nonan-8-yl)urea

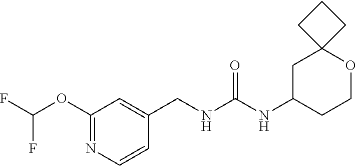 [SMILES: O=C(NCc1ccnc(OC(F)F)c1)NC1CCOC2(CCC2)C1]

The title compound was obtained in analogy to Example 2 as an off-white solid (34% yield) using (2-(difluoromethoxy)pyridin-4-yl)methanamine, 5-oxaspiro[3.5]nonan-8-amine and 1,1′-carbonyldiimidazole.

MS (m/z): 342.2 [M+H]+.

### Example 18: 1-[[2-(difluoromethoxy)pyridin-4-yl]methyl]-3-[2-(hydroxymethyl)spiro[3.3]heptan-2-yl]urea

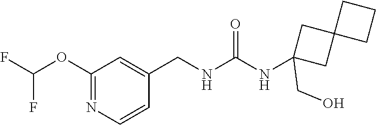 [SMILES: O=C(NCc1ccnc(OC(F)F)c1)NC1(CO)CC2(CCC2)C1]

The title compound was obtained in analogy to Example 8 as an off-white solid (63% yield) using (2-(difluoromethoxy)pyridin-4-yl)methanamine, {2-aminospiro[3.3]heptan-2-yl}methanol hydrochloride and para-nitrophenyl chloroformate.

MS (m/z): 342.2 [M+H]+.

### Example 19: 1-[[2-(difluoromethoxy)pyridin-4-yl]methyl]-3-(2,2-difluorospiro[2.4]heptan-6-yl)urea

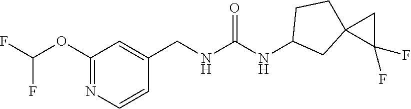 [SMILES: O=C(NCc1ccnc(OC(F)F)c1)NC1CCC2(C1)CC2(F)F]

The title compound was obtained in analogy to Example 8 as a cream colored solid (75% yield) using (2-(difluoromethoxy)pyridin-4-yl)methanamine, 1,1-difluorospiro[2.4]heptan-5-amine hydrochloride and para-nitrophenyl chloroformate.

MS (m/z): 348.1 [M+H]+.

### Example 20: 1-[[2-(difluoromethoxy)pyridin-4-yl]methyl]-3-(1-oxaspiro[3.5]nonan-3-yl)urea

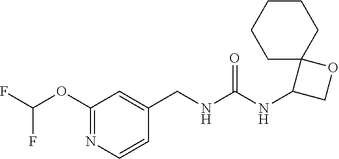 [SMILES: O=C(NCc1ccnc(OC(F)F)c1)NC1COC12CCCCC2]

The title compound was obtained in analogy to Example 8 as a colorless gum (89% yield) using (2-(difluoromethoxy)pyridin-4-yl)methanamine, 1-oxaspiro[3.5]nonan-3-amine and para-nitrophenyl chloroformate.

MS (m/z): 342.2 [M+H]+.

### Example 21: 1-[[2-(difluoromethoxy)pyridin-4-yl]methyl]-3-(4,4-difluoro-6-oxaspiro[4.5]decan-9-yl)urea

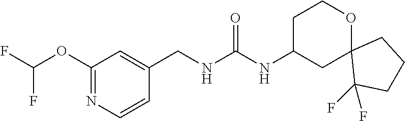 [SMILES: O=C(NCc1ccnc(OC(F)F)c1)NC1CCOC2(CCCC2(F)F)C1]

The title compound was obtained in analogy to Example 8 as a colorless gum (61% yield) using (2-(difluoromethoxy)pyridin-4-yl)methanamine, 1,1-difluoro-6-oxaspiro[4.5]decan-9-amine hydrochloride and para-nitrophenyl chloroformate.

MS (m/z): 392.2 [M+H]+.

### Example 22: 1-[[2-(difluoromethoxy)pyridin-4-yl]methyl]-3-(5-oxaspiro[3.4]octan-7-yl)urea

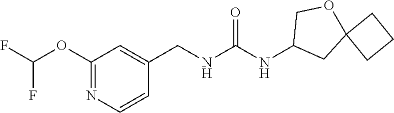 [SMILES: O=C(NCc1ccnc(OC(F)F)c1)NC1COC2(CCC2)C1]

The title compound was obtained in analogy to Example 8 as a white solid (78% yield) using (2-(difluoromethoxy)pyridin-4-yl)methanamine, 5-oxaspiro[3.4]octan-7-amine hydrochloride and para-nitrophenyl Chloroformate.

MS (m/z): 328.1 [M+H]+.

### Example 23: 1-[[2-(difluoromethoxy)pyridin-4-yl]methyl]-3-spiro[2.3]hexan-2-ylurea

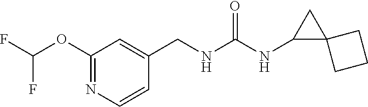 [SMILES: O=C(NCc1ccnc(OC(F)F)c1)NC1CC12CCC2]

The title compound was obtained in analogy to Example 8 as a colorless gum (67% yield) using (2-(difluoromethoxy)pyridin-4-yl)methanamine, spiro[2.3]hexan-1-amine hydrochloride and 4-nitrophenyl chloroformate.

MS (m/z): 298.1 [M+H]+.

### Example 24: 1-[[2-(difluoromethoxy)pyridin-4-yl]methyl]-3-(2,2-difluorospiro[2.3]hexan-1-yl)urea

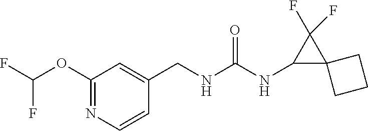 [SMILES: O=C(NCc1ccnc(OC(F)F)c1)NC1C(F)(F)C12CCC2]

The title compound was obtained in analogy to Example 8 as a cream colored solid (33% yield) using (2-(difluoromethoxy)pyridin-4-yl)methanamine, 2,2-difluorospiro[2.3]hexan-1-amine hydrochloride and 4-nitrophenyl chloroformate.

MS (m/z): 334.1 [M+H]+.

### Example 25: 1-[[2-(difluoromethoxy)pyridin-4-yl]methyl]-3-[rac-(2R,3R)-3-hydroxyspiro[3.3]heptan-2-yl]urea

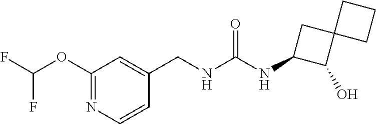 [SMILES: O=C(NCc1ccnc(OC(F)F)c1)N[C@H]1CC2(CCC2)[C@@H]1O]

The title compound was obtained in analogy to Example 8 as a white solid (11% yield) using (2-(difluoromethoxy)pyridin-4-yl)methanamine, 2-aminospiro[3.3]heptan-1-ol hydrochloride and para-nitrophenyl chloroformate. Purification by preparative HPLC also provided example 26.

MS (m/z): 328 [M+H]+.

### Example 26: 1-[[2-(difluoromethoxy)pyridin-4-yl]methyl]-3-[rac-(2R,3S)-3-hydroxyspiro[3.3]heptant-2-yl]urea

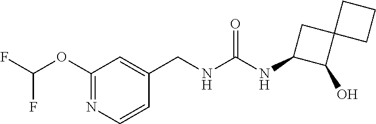 [SMILES: O=C(NCc1ccnc(OC(F)F)c1)N[C@H]1CC2(CCC2)[C@H]1O]

The title compound is the cis-racemate that was isolated from the purification of Example 25. It was obtained as a white solid (53% yield).

MS (m/z): 328 [M+H]+.

### Example 27: 1-[[2-(difluoromethoxy)pyridin-4-yl]methyl]-3-[(8R)-5-oxaspiro[3.5]nonan-8-yl]urea

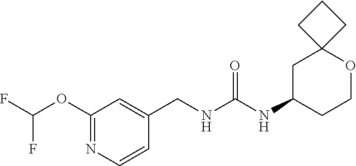 [SMILES: O=C(NCc1ccnc(OC(F)F)c1)N[C@@H]1CCOC2(CCC2)C1]

1-[[2-(difluoromethoxy)pyridin-4-yl]methyl]-3-[5-oxaspiro[3.5]nonan-8-yl]urea was obtained in analogy to Example 8 as a white solid (76% yield) using (2-(difluoromethoxy)pyridin-4-yl)methanamine, 5-oxaspiro[3.5]nonan-8-amine and para-nitrophenyl chloroformate. Purification by chiral HPLC (Cellulose-4 column, eluted with water:acetonitrile 55:45) gave the title product (32.8% yield, >99% ee) as a white solid.

MS (m/z): 342.2 [M+H]+.

### Example 28: 1-[[2-(difluoromethoxy)pyridin-4-yl]methyl]-3-[(2R,3S)-3-hydroxyspiro[3.3]heptan-2-yl]urea

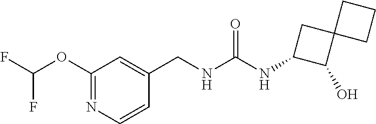 [SMILES: O=C(NCc1ccnc(OC(F)F)c1)N[C@@H]1CC2(CCC2)[C@@H]1O]

The title product was obtained as a white solid by chiral HPLC purification of 1-[[2-(difluoromethoxy)pyridin-4-yl]methyl]-3-[rac-(2R,3S)-3-hydroxyspiro[3.3]heptan-2-yl]urea (Example 26) (Cellulose-4 column, eluted with water:acetonitrile 55:45, 23.9% yield, 100% de).

MS (m/z): 328.1 [M+H]+.

### Example 29: 1-[[2-(2,3-difluoropropoxy)pyridin-4-yl]methyl]-3-spiro[3.3]heptan-2-ylurea

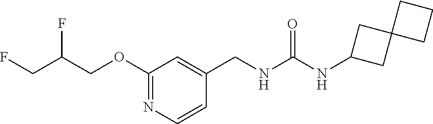 [SMILES: O=C(NCc1ccnc(OCC(F)CF)c1)NC1CC2(CCC2)C1]

The title compound was obtained in analogy to Example 5 as a while solid using (2-chloropyridin-4-yl)methanamine hydrochloride, 2,3-difluoropropan-1-ol, spiro[3.3]heptan-2-amine hydrochloride, and para-nitrophenyl chloroformate.

MS (m/z): 340.2 [M+H]+.

### Example 30: 1-[[2-(difluoromethoxy)pyridin-4-yl]methyl]-3-(1-oxaspiro[4.5]decan-3-yl)urea

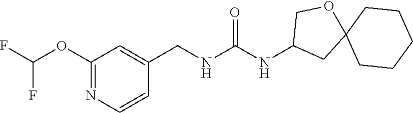 [SMILES: O=C(NCc1ccnc(OC(F)F)c1)NC1COC2(CCCCC2)C1]

The title compound was obtained in analogy to Example 8 as a colorless gum (90.0% yield) using (2-(difluoromethoxy)pyridin-4-yl)methanamine, 1-oxaspiro[4.5]decan-3-amine hydrochloride, and para-nitrophenyl chloroformate.

MS (m/z): 356.2 [M+H]+.

### Example 31: 1-[[2-(difluoromethoxy)pyridin-4-yl]methyl]-3-(2,2-difluorospiro[2.5]octan-6-yl)urea

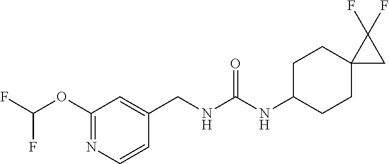 [SMILES: O=C(NCc1ccnc(OC(F)F)c1)NC1CCC2(CC1)CC2(F)F]

The title compound was obtained in analogy to Example 8 as a white solid (85.6% yield) using (2-(difluoromethoxy)pyridin-4-yl)methanamine, 1,1-difluorospiro[2.5]octan-6-amine hydrochloride and para-nitrophenyl chloroformate.

MS (m/z): 362.2 [M+H]+.

### Example 32: 1-[[2-(difluoromethoxy)pyridin-4-yl]methyl]-3-(5-oxaspiro[3.5]nonan-7-yl)urea

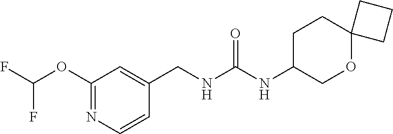 [SMILES: O=C(NCc1ccnc(OC(F)F)c1)NC1CCC2(CCC2)OC1]

The title compound was obtained in analogy to Example 8 as a white solid (68.4% yield) using (2-(difluoromethoxy)pyridin-4-yl)methanamine, 5-oxaspiro[3.5]nonan-7-amine and para-nitrophenyl chloroformate.

MS (m/z): 342.2 [M+H]+.

### Biological Experiments

The potency of Kv7 potentiators was determined using the SyncroPatch 384 (Nanion) high throughput electrophysiology platform.

### Example 33 Cell Culture

CHO cells stably expressing either human Kv7.2, Kv7.4 or Kv7.5/7.3 under a constitutive CMV promoter were used for these studies. Cells were maintained in F12 Hams+1 mM L-glutamine (Hyclone) supplemented with 10% FBS (Sigma), 0.3×NEAA (Non-essential Gamino-acids) and 400 ug/ml G418 at 37° C. in 5% CO₂. Cells were cultured in T-225 flasks (Nunc) for 2-3 days to reach 85-95% confluence prior to electrophysiological recording.

### Example 34 Solutions

Solutions were of the following composition:

Earle's balanced salt solution (in mM): 135 NaCl, 5.4 KCl, 5 Glucose, 2 CaCl₂, 1 MgCl₂, 5 HEPES, pH 7.4. Seal enhancer solution (in mM): 90 NaCl, 3 KCl, 35 CaCl₂, 10 MgCl₂, 10 HEPES, pH 7.4. Extracellular recording solution (in mM): 71 NaCl, 70 NMDG, 13 KCl, 5 Glucose, 2 CaCl₂, 1 MgCl₂, 10 HEPES, pH 7.4. Intracellular recording solution (in mM): 130 KF, 20 KCl, 4 EGTA, 10 HEPES, 2 EDTA, 0.01 Escin, pH 7.2.

### Example 35 Cell Preparation

Cells were harvested for electrophysiological recording upon reaching appropriate confluence. Cells were first washed in DPBS (Hyclone, Cat #SH30028.03) and then 2 ml of Accutase (MP Biomedicals #1000449) was added at 28° C. until ˜90% of cells were suspended. F12 HAM's media+1 mM L-glutamine (Hyclone, SH30026.02) was then added to the flask to dilute the accutase. Cells were then triturated until a single cell suspension was achieved, a cell count was performed, and cells were centrifuged for 2 min. at 1,000 rpm. Media was then aspirated, and cells were resuspended in Earle's balanced salt solution to a concentration of 0.75×10⁶ cells/ml and allowed to recover for 25 min at 10° C.

### Example 36 SyncroPatch Recording

At the beginning of each assay, 20 μl of cell suspension was dispensed into each well of a multi-hole 384-well SyncroPatch chip by the onboard pipettor. Cell sealing was initiated, and seal enhancer solution was added to facilitate seal formation. Upon completion of sealing, cells were washed 3 times with extracellular recording solution and the assay voltage protocol was started. Human Kv7.2, Kv7.4 or Kv7.5/7.3 channels were evaluated using a voltage protocol in which cells were voltage-clamped at a holding potential of −60 mV. Potassium currents were continuously activated with a series of three voltage steps to −30 mV for 3 seconds, 40 mV for 1 second and −90 mV for 4 seconds with 12 seconds between successive voltage sweeps. Potassium currents were measured from the −90 mV repolarizing step. Baseline current was assessed for 3.5 minutes prior to the addition of 5.6 μM zinc pyrithione (1 μM for Kv7.4). Kv7.2, Kv7.4 or Kv7.5/7.3 current in the presence of zinc pyrithione was acquired over five minutes to allow channels to reach steady state activity prior to addition of test agents. Channel activity was monitored for three minutes preceding the addition of 30 μM ML-213 (3 minutes) to achieve maximum activation. 150 mM TEA with 10 μM XE-991 was applied for 2 minutes to measure the leak current during maximum inhibition of Kv7.2, Kv7.4 or Kv7.5/7.3 channels.

### Example 37 Data Analysis

Data were collected on the SyncroPatch platform using PatchControl software (Nanion) and processed and analyzed using DataControl Software (Nanion). Percent activation was calculated from potassium current as follows: The average current for the 5 sweeps in the presence of zinc pyrithione immediately preceding test agent addition was taken as the ‘control’ data. Likewise, the average potassium current in the presence of test agents 5 sweeps immediately preceding addition of 30 μM ML-213 was determined for ‘drug’ data. The average potassium current 5 sweeps immediately prior to addition of 150 mM TEA+10 μM XE-991 was determined for ‘max activation’ data while average currents from 5 sweeps immediately following addition of TEA was determined for ‘max inhibition’ data. Percent activation for each of the 384-wells of a sealchip was calculated as ((‘drug’−‘max inhibition’)/(‘max activation’−‘max inhibition’))*100 with Pipeline Pilot (Accelrys). Percent activation was plotted as a function of concentration and concentration-response curves were fitted with a logistic equation {Y is Bottom+(Top−Bottom)/(1+10\hat{ }((LogEC50−X)*HillSlope))} for determination of the EC₅₀ (IDBS ABASE). EC₅₀ values for human Kv7.2 are provided in Table 1; EC₅₀ values for human Kv7.5/7.3 and Kv7.5/7.3 to Kv7.2 selectivity ratios are provided in Table A; EC₅₀ values for human Kv7.4 and Kv7.4 to Kv7.2 selectivity ratios are provided in Table C.

### Example 38 Human Liver Microsomal Clearance

Pooled mixed gender human liver microsomes were purchased from BioIVT at a concentration of 20 mg protein/mL and stored at −80° C. Incubations were conducted in V-bottomed 350 μL polypropylene 96-well plates. Compounds were diluted in phosphate buffer (final concentrations 0.5 μM compound, 0.5% DMSO) and mixed with diluted liver microsomes (fc 0.25 mg/mL). This mixture was aliquoted onto the assay plate (six wells per compound) and pre-warmed at 37° C. for ten minutes. Addition of NADPH (fc 0.5 mM, final incubation volume 100 μL) to each well started the time course. Reactions were stopped at 0.5, 3, 5, 10, 20 & 30 minutes by addition of 150 μL acetonitrile containing internal standard (IS; 0.125 μg/mL daidzein) and stopped samples were removed to a clean polypropylene plate. At the end of the assay the plate was refrigerated for an hour and then centrifuged at 3000 rpm for 10 minutes at 4° C. A sample of the supernatant was removed to a fresh plate and diluted 10-fold with 50:50 acetonitrile:water, then heat sealed. Analysis was by liquid chromatography coupled with tandem mass spectrometry (LC-MS/MS) for analyte and IS levels. Ratios of the analyte and IS peak areas were generated for results calculation (IS ratio). Dextromethorphan and verapamil were included in each experiment as controls for high clearance compounds. IS ratio data were converted into natural log values which were plotted against time and a linear regression fit applied. The slope of the line was returned and converted into the elimination constant (kₑₗ) by multiplying by −1. The elimination constant was used to calculate the in vitro CLᵢₙₜ: in vitro CLᵢₙₜ (μL/min/mg protein) is kₑₗ* (1000/protein conc in mg/mL). Human liver microsomal intrinsic clearance Clint values are provided in Table B.

### Example 39

A compound of formula (I), or a solvate or a pharmaceutically acceptable salt thereof can be used in a manner known per se as the active ingredient for the production of tablets of the following composition:

Per tablet

| | | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 40

A compound of formula (I), or a solvate or a pharmaceutically acceptable salt thereof, can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A compound of formula (I): 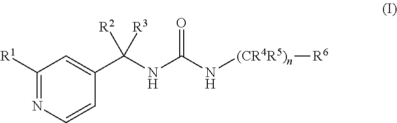
wherein
R¹ is selected from a 5-6 membered heteroaryl, haloC₁₋₆alkoxy, C₃₋₅cycloalkyl-C₁₋₆alkoxy, cyano, haloC₁₋₆alkyl, halogen, C₁₋₆alkyl, C₁₋₆alkoxy, and C₃₋₅heterocyclyloxy, wherein such heteroaryl, cycloalkylalkoxy, and heterocycyloxy are optionally substituted with one, two, or three substituents independently selected from halogen, haloC₁₋₆alkyl, C₁₋₆alkyl, and C₁₋₆alkoxy;
R² is selected from H, C₁₋₆alkyl, and hydroxyC₁₋₆alkyl;
R³ is H or C₁₋₆alkyl;
R⁴ is H or halogen;
R⁵ is H or halogen;
n is 0 or 1; and
R⁶ is a 7-11 membered spirocylic cyloalkyl or 7-11 membered oxo-spiro-heterocycloalkyl optionally substituted with one, two, or three substituents independently selected from halogen, C₁₋₆ alkyl, hydroxy, and hydroxyC₁₋₆alkyl;
or a solvate or a pharmaceutically acceptable salt thereof.

2. A compound according to claim 1, wherein R¹ is selected from 5 membered heteroaryl, haloC₁₋₆alkoxy, and haloC₁₋₆alkyl.

3. A compound according to claim 2, wherein R¹ is unsubstituted pyrazolyl.

4. A compound according to claim 2, wherein R¹ is haloC₁₋₆alkoxy selected from FCH₂CHFCH₂O—, CHF₂O—, CH₃CF₂O—, CH₃CFHCH₂O—, and CF₃CH₂O—.

5. A compound according to claim 4, wherein R¹ is haloC₁₋₆alkyl selected from CHF₂—, CF₃—, FCH₂CFHCH₂—, and CF₃CH₂—.

6. A compound according to any one of claims 1-5, wherein (i) both R² and R³ are H, or (ii) R² is HOCH₂— and R³ is H.

7. A compound according to any one of claims 1-6, wherein n is 0.

8. A compound according to any one of claims 1-6, wherein n is 1 and one or both of R⁴ and R⁵ are halogen.

9. A compound according to claim 8, wherein n is 1 and both of R⁴ and R⁵ are halogen.

10. A compound according to claim 9, wherein halogen is F—.

11. A compound according to any one claims 1-10, wherein R⁶ is substituted with one or two substituents independently selected from F—, hydroxy, and hydroxyC₁₋₆alkyl.

12. A compound according to any one of claims 1-11, wherein R⁶ is 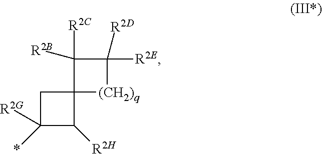
and wherein R^{2B}, R^{2C}, R^{2D}; R^{2E}, R^{2G} and R^{2H} are independently selected from H, halogen, C₁₋₆alkyl, hydroxy, and hydroxyC₁₋₆alkyl, and q is 0 or 1, provided that one of R^{2G} and R^{2H} is not hydrogen.

13. A compound according to any one of claims 1-11, wherein R⁶ is a 7-11 membered oxa-spiro-heterocycloalkyl with the structure: 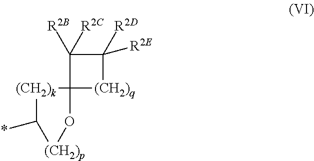
wherein
p is 1, or 2;
q is 1, 2, or 3;
k is 0, or 1
R^{2B}, R^{2C}; R^{2D}, and R^{2E} are independently selected from H, halogen, C₁₋₆alkyl, hydroxyl, and hydroxyC₁₋₆alkyl.

14. A compound according to any one of claims 1-11, wherein R⁶ is a 7-11 membered oxa-spiro-heterocycloalkyl with the structure: 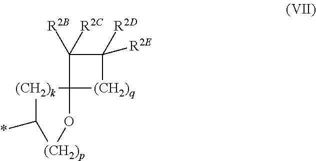
wherein
p is 1, or 2;
q is 1, 2, or 3;
k is 0, or 1
R^{2B}, R^{2C}; R^{2D}, and R^{2E} are independently selected from H and halogen

15. A compound according to any one claims 1-14, wherein R⁶ is selected from a group consisting of:

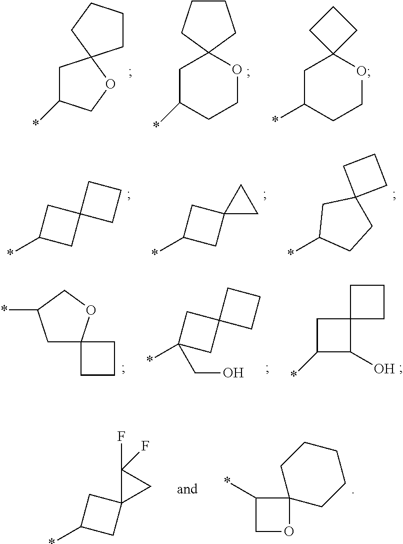

16. A compound according to claim 15, wherein R⁶ is selected from a group consisting of:

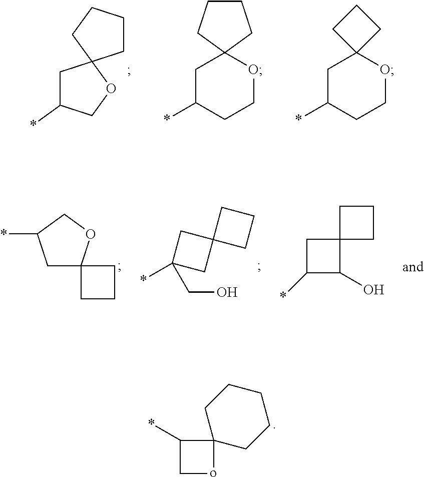

17. A compound according to claim 15, wherein R⁶ is selected from a group consisting of:

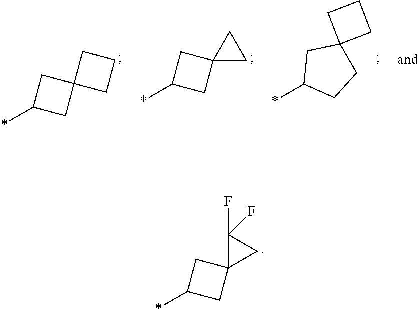

18. A compound according to any one of claims 1-17, wherein R⁶ is not selected from:

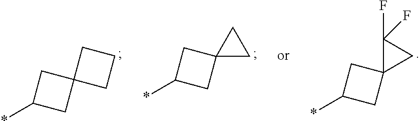

19. A compound according to any one of claims 1-18, wherein R¹ is CHF₂O—, R² and are R³ are H, n is 0 and R⁶ is selected from the group consisting of:

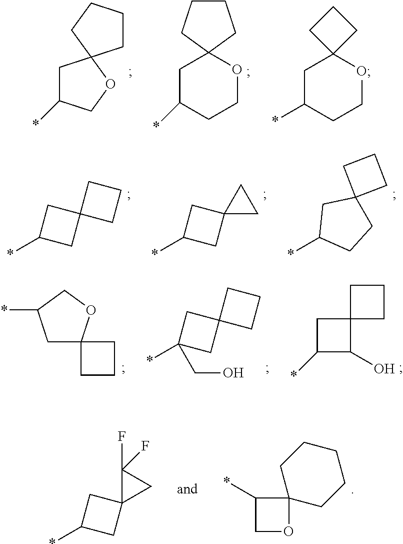

20. A compound according to any one of claims 1-19, selected from the group consisting of:

| | | |
|---|---|---|
| 1 | 1-(1- oxaspiro[4.4]nonan- 3-yl)-3-[(2- pyrazol-1- ylpyridin-4- yl)methyl]urea | 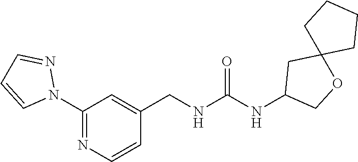 [SMILES: O=C(NCc1ccnc(-n2cccn2)c1)NC1COC2(CCCC2)C1] |
| 2 | 1-(6- oxaspiro[4.5]decan- 9-yl)-3-[(2- pyrazol-1- ylpyridin-4- yl)methyl]urea | 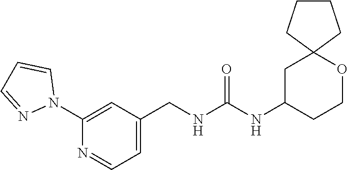 [SMILES: O=C(NCc1ccnc(-n2cccn2)c1)NC1CCOC2(CCCC2)C1] |
| 3 | 1-[[2- (difluoromethoxy) pyridin-4- yl]methyl]-3-(1- oxaspiro[4.4]nonan- 3-yl)urea | 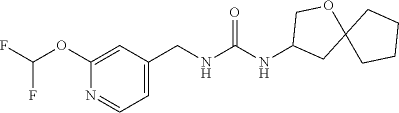 [SMILES: O=C(NCc1ccnc(OC(F)F)c1)NC1COC2(CCCC2)C1] |
| 4 | 1-[[2- (difluoromethoxy) pyridin-4- yl]methyl]-3- spiro[3.3]heptan- 2-ylurea | 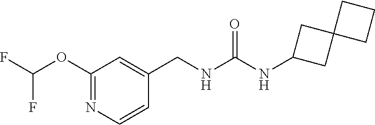 [SMILES: O=C(NCc1ccnc(OC(F)F)c1)NC1CC2(CCC2)C1] |
| 5 | 1-[[2-(2,2- difluoropropoxy) pyridin-4- yl]methyl]-3- spiro[3.3]heptan- 2-ylurea | 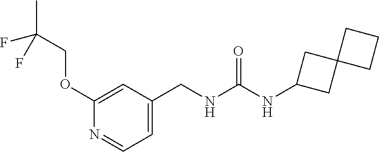 [SMILES: CC(F)(F)COc1cc(CNC(=O)NC2CC3(CCC3)C2)ccn1] |
| 6 | 1-[[2-(2- fluoropropoxy) pyridin-4-yl] methyl]- 3- spiro[3.3]heptan- 2-ylurea | 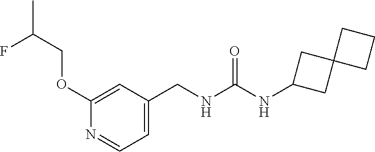 [SMILES: CC(F)COc1cc(CNC(=O)NC2CC3(CCC3)C2)ccn1] |
| 7 | 1-[[2- (cyclopropylmethoxy) pyridin-4- yl]methyl]-3- spiro[3.3]heptan- 2-ylurea | 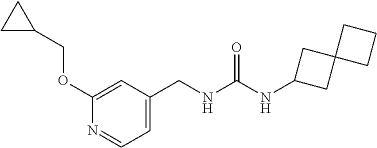 [SMILES: O=C(NCc1ccnc(OCC2CC2)c1)NC1CC2(CCC2)C1] |
| 8 | 1-[[2- (difluoromethoxy) pyridin-4- yl]methyl]-3- spiro[2.3]hexan-5- ylurea | 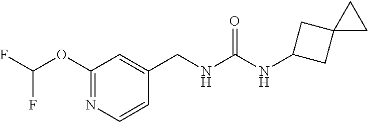 [SMILES: O=C(NCc1ccnc(OC(F)F)c1)NC1CC2(CC2)C1] |
| 9 | 1-[[2-(1,1- difluoroethyl) pyridin-4-yl]methyl]- 3- spiro[3.3]heptan- 2-ylurea | 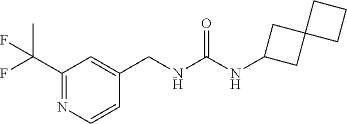 [SMILES: CC(F)(F)c1cc(CNC(=O)NC2CC3(CCC3)C2)ccn1] |
| 10 | 1- spiro[3.3]heptan- 2-yl-3-[[2- (trifluoromethyl) pyridin-4- yl]methyl]urea | 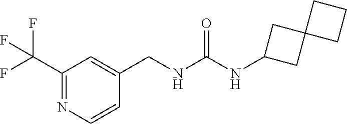 [SMILES: O=C(NCc1ccnc(C(F)(F)F)c1)NC1CC2(CCC2)C1] |
| 11 | 1-[[2- (difluoromethoxy) pyridin-4- yl]methyl]-3- spiro[3.4]octan-7- ylurea | 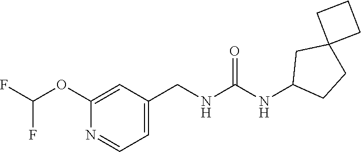 [SMILES: O=C(NCc1ccnc(OC(F)F)c1)NC1CCC2(CCC2)C1] |
| 12 | 1-spiro[2.3]hexan- 5-yl-3-[[2-(2,2,2- trifluoroethoxy) pyridin-4- yl]methyl]urea | 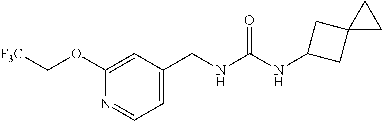 [SMILES: O=C(NCc1ccnc(OCC(F)(F)F)c1)NC1CC2(CC2)C1] |
| 13 | 1-[2-hydroxy-1- [2-(2,2,2- trifluoroethoxy) pyridin-4-yl]ethyl]- 3- spiro[3.3]heptan- 2-ylurea | 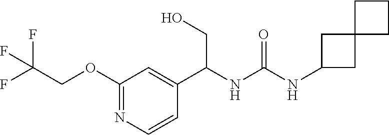 [SMILES: O=C(NC1CC2(CCC2)C1)NC(CO)c1ccnc(OCC(F)(F)F)c1] |
| 14 | 1-[2- (difluoromethoxy) pyridin-4- yl]methyl]-3-(6- oxaspiro[4.5]decan- 9-yl)urea | 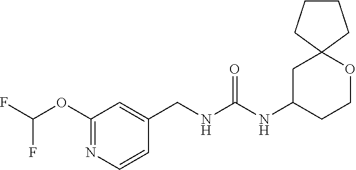 [SMILES: O=C(NCc1ccnc(OC(F)F)c1)NC1CCOC2(CCCC2)C1] |
| 15 | 1-[[2- (difluoromethoxy) pyridin-4- yl]methyl]-3-(2,2- difluorospiro[2.3] hexan-5-yl)urea | 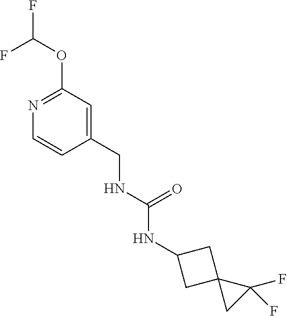 [SMILES: O=C(NCc1ccnc(OC(F)F)c1)NC1CC2(C1)CC2(F)F] |
| 16 | 1-[[2- (difluoromethoxy) pyridin-4- yl]methyl]-3-(2- fluorospiro[3.3] heptan-6-yl)urea | 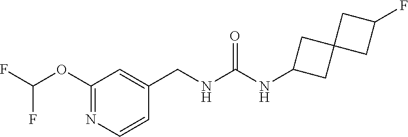 [SMILES: O=C(NCc1ccnc(OC(F)F)c1)NC1CC2(CC(F)C2)C1] |
| 17 | 1-[[2- (difluoromethoxy) pyridin-4- yl]methyl]-3-(5- oxaspiro[3.5]nonan- 8-yl)urea | 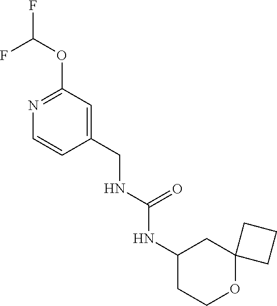 [SMILES: O=C(NCc1ccnc(OC(F)F)c1)NC1CCOC2(CCC2)C1] |
| 18 | 1-[[2- (difluoromethoxy) pyridin-4- yl]methyl]-3-[2- (hydroxymethyl) spiro[3.3]heptan-2- yl]urea | 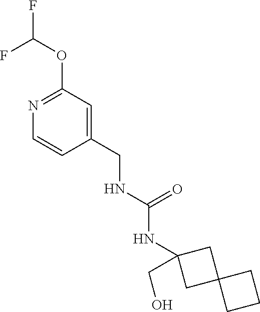 [SMILES: O=C(NCc1ccnc(OC(F)F)c1)NC1(CO)CC2(CCC2)C1] |
| 19 | 1-[[2- (difluoromethoxy) pyridin-4- yl]methyl]-3-(2,2- difluorospiro[2.4] heptan-6-yl)urea | 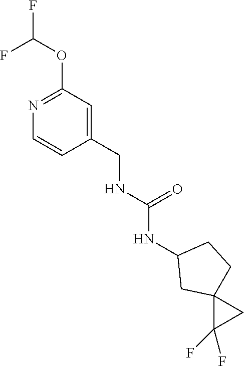 [SMILES: O=C(NCc1ccnc(OC(F)F)c1)NC1CCC2(C1)CC2(F)F] |
| 20 | 1-[[2- (difluoromethoxy) pyridin-4- yl]methyl]-3-(1- oxaspiro[3.5]nonan- 3-yl)urea | 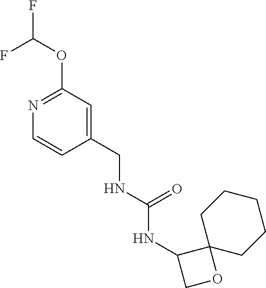 [SMILES: O=C(NCc1ccnc(OC(F)F)c1)NC1COC12CCCCC2] |
| 21 | 1-[[2- (difluoromethoxy) pyridin-4- yl]methyl]-3-(4,4- difluoro-6- oxaspiro[4.5]decan- 9-yl)urea | 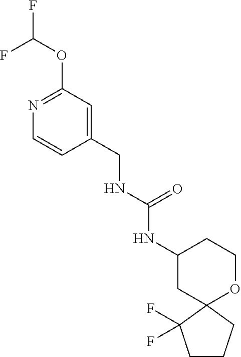 [SMILES: O=C(NCc1ccnc(OC(F)F)c1)NC1CCOC2(CCCC2(F)F)C1] |
| 22 | 1-[[2- (difluoromethoxy) pyridin-4- yl]methyl]-3-(5- oxaspiro[3.4]octan- 7-yl)urea | 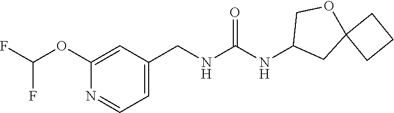 [SMILES: O=C(NCc1ccnc(OC(F)F)c1)NC1COC2(CCC2)C1] |
| 23 | 1-[[2- (difluoromethoxy) pyridin-4- yl]methyl]-3- spiro[2.3]hexan-2- ylurea | 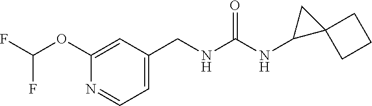 [SMILES: O=C(NCc1ccnc(OC(F)F)c1)NC1CC12CCC2] |
| 24 | 1-[[2- (difluoromethoxy) pyridin-4- yl]methyl]-3-(2,2- difluorospiro[2.3] hexan-1-yl)urea | 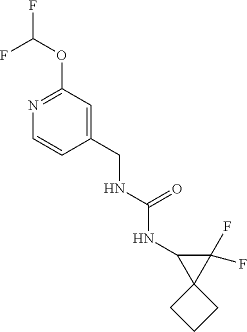 [SMILES: O=C(NCc1ccnc(OC(F)F)c1)NC1C(F)(F)C12CCC2] |
| 25 | 1-[[2- (difluoromethoxy) pyridin-4- yl]methyl]-3-[rac- (2R,3R)-3- hydroxyspiro[3.3] heptan-2-yl]urea | 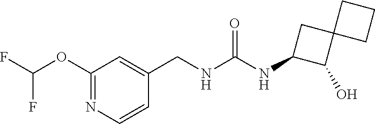 [SMILES: O=C(NCc1ccnc(OC(F)F)c1)N[C@H]1CC2(CCC2)[C@@H]1O] |
| 26 | 1-[[2- (difluoromethoxy) pyridin-4- yl]methyl]-3-[rac- (2R,3S)-3- hydroxyspiro[3.3] heptan-2-yl]urea | 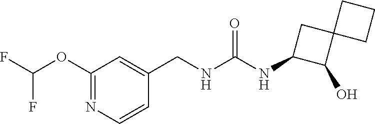 [SMILES: O=C(NCc1ccnc(OC(F)F)c1)N[C@H]1CC2(CCC2)[C@H]1O] |
| 27 | 1-[[2- (difluoromethoxy) pyridin-4- yl]methyl]-3- [(8R)-5- oxaspiro[3.5]nonan- 8-yl]urea | 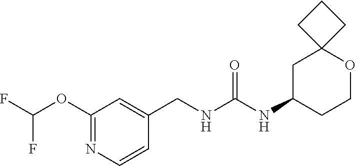 [SMILES: O=C(NCc1ccnc(OC(F)F)c1)N[C@@H]1CCOC2(CCC2)C1] |
| 28 | 1-[[2- (difluoromethoxy) pyridin-4- yl]methyl]-3- [(2R,3S)-3- hydroxyspiro[3.3] heptan-2-yl]urea | 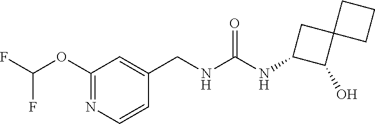 [SMILES: O=C(NCc1ccnc(OC(F)F)c1)N[C@@H]1CC2(CCC2)[C@@H]1O] |
| 29 | 1-[[2-(2,3- difluoropropoxy) pyridin-4- yl]methyl]-3- spiro[3.3]heptan- 2-ylurea | 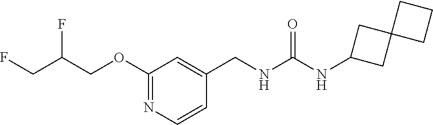 [SMILES: O=C(NCc1ccnc(OCC(F)CF)c1)NC1CC2(CCC2)C1] |
| 30 | 1-[[2- (difluoromethoxy) pyridin-4- yl]methyl]-3-(1- oxaspiro[4.5]decan- 3-yl)urea | 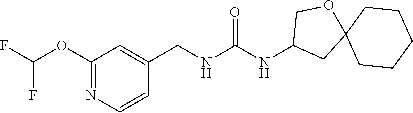 [SMILES: O=C(NCc1ccnc(OC(F)F)c1)NC1COC2(CCCCC2)C1] |
| 31 | 1-[[2- (difluoromethoxy) pyridin-4- yl]methyl]-3-(2,2- difluorospiro[2.5] octan-6-yl)urea | 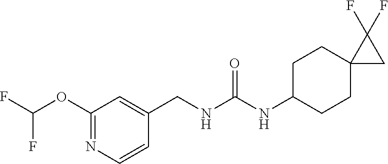 [SMILES: O=C(NCc1ccnc(OC(F)F)c1)NC1CCC2(CC1)CC2(F)F] |
| 32 | 1-[[2- (difluoromethoxy) pyridin-4- yl]methyl]-3-(5- oxaspiro[3.5]nonan- 7-yl)urea | 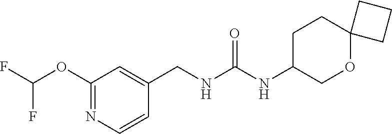 [SMILES: O=C(NCc1ccnc(OC(F)F)c1)NC1CCC2(CCC2)OC1] |

21. A compound of formula (I), or a solvate or a pharmaceutically acceptable salt thereof, according to any one of claims 1-20, wherein such compounds show Kv7.2 EC₅₀: <3 μM.

22. A pharmaceutical composition comprising a compound of formula (I), or a solvate or a pharmaceutically acceptable salt thereof according to any one of claims 1-20, for use in the therapeutic and/or prophylactic treatment of a disorder, disease, or disability associated with Kv7.2 in a subject in need thereof.

23. A compound of formula (I), or a solvate or a pharmaceutically acceptable salt thereof according to any one of claims 1-21; or a pharmaceutical composition according to claim 22; for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

24. The compound or pharmaceutical composition for use according to claim 23, wherein the disorder, disease, or disability is a behavioral disorder which is Attention Deficit Hyperactivity Disorder (ADHD).

25. The compound or pharmaceutical composition for use according to claim 23, wherein the disorder, disease, or disability is a mood disorder which is depression.

26. The compound or pharmaceutical composition for use according to claim 23, wherein the disorder, disease, or disability is a neurodevelopment disorder selected from autism spectrum disorder (ASD) and syndromic developmental disorders.

27. The compound or pharmaceutical composition for use according to claim 23, wherein the disorder, disease, or disability is a syndromic developmental disorder selected from Dup15q syndrome (Dup15q), Fragile X syndrome (FXS) and Angelman syndrome.

28. The compound or pharmaceutical composition for use according to claim 23, wherein the disorder, disease, or disability is an epilepsy selected from broad pediatric epilepsy, West syndrome, Ohtahara syndrome, and epileptic encephalopathy.

29. The compound or pharmaceutical composition for use according to claim 23, wherein disorder, disease, or disability is a neurodegenerative disease selected from Alzheimer's disease, and motor neuron diseases.

30. The compound or pharmaceutical compositions for use according to any one of claims 23-29, for systemic or local administration such as oral, nasal, parenteral (as by intravenous (both bolus and infusion), intramuscular, or subcutaneous injection), transdermal, vaginal, buccal, rectal, or topical administration modes, intracisternally, intraperitoneally, as an oral or nasal spray, or as a liquid aerosol or dry powder for inhalation.

31. A compound according to any one of claims 1-21, or a pharmaceutical composition according to claim 22 for use in therapy.

32. A compound according to any one of claims 1-21, or a pharmaceutical composition according to claim 22, or a pharmaceutical composition for use according to any one of claims 23-29, for the manufacture of a medicament for use in the therapeutic and/or prophylactic treatment of a disorder, disease, or disability associated with Kv7.2.

33. A method for the therapeutic and/or prophylactic treatment of a disorder, disease, or disability associated with Kv7.2, which method comprises administering a therapeutically effective amount of a compound according to any one of claims 1-21, or a pharmaceutical composition according to claim 22 for use according to any one of claims 23-29.

34. A kit for use in the therapeutic and/or prophylactic treatment of a disorder, disease, or disability associated with Kv7.2 comprising:
a) a compound according to any one of claims 1-21, or a pharmaceutical composition according to 22; and
b) instructions for use.

35. The invention as hereinbefore described.
